SEQUENCE LISTING (41 sequences)

SEQ ID NO: 1 - DNA, 7434 bp, Artificial Sequence
  pSIN-OM-1.8-GalT1
cgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtacctcaacatgaaagaacctgcacttcctggtacttcactgcaaagagcatgtagactgctggtagcattttgcgccctgcacctgagcgcaaccctgctctactacctggctggatccagcctgactccaccccgctctccagaacctccccctcggaggccgcctccagccaacctctccctgccaccctcccggcctcctcctccccctgcggctcgcccccgcccaggacctgtttctgcacaaccccggaacctgccagattctgcaccatctggactgtgccccgatccaagtccactgctcgttggtcctctgcgggtggagtttagtcagccagtgaacctggaggaagtggcttctaccaatccggaggtcagggaaggagggagattcgccccaaaggactgcaaagcgctccagaaggtggctattattatccccttcaggaacagagaggagcacctgaagtattggctgtactacatgcacccgattcttcagagacagcaattggactatggggtctatgtgattaatcaagacggcgatgaagaatttaacagagctaaactgcttaatgtcggtttcactgaggcactcaaggaatacgattatgattgctttgtgttttccgatgtggatctgattcctatggacgaccgtaacacatataagtgctatagtcaaccacgtcacctgagtgtgtcaatggacaagtttggctttaggctgccgtataaccagtatttcggaggagtttcagcattgagtaaagaacagtttacaaaaatcaacgggttcccaaataactactgggggtggggcggagaggacgacgacatctacaacagactggtttttaaggggatggggatttcccgcccggatgcagtaataggcaagtgtcgtatgatacgccatagcagggatagaaagaacgaacccaaccctgagcgctttgaccggattgcacatacaagagaaactatgtcatctgatggacttaactctctttcatatgaggtgctgagaacagatcggttccccctgtacactagaatcacagtagatatcggggcacctgggtcataagcctaaagtctagtatggggattggtggcgacgactcctggagcccgtcagtatcggcggaattcggtaccggatccc

SEQ ID NO: 2 - DNA, 7545 bp, Artificial Sequence
  pSIN-OM-1.8-SialT3
cgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccatactggccaagtcctgcccagctgtcagcctgctgaccctctgcagttcaggaccatgaaacgtggcactgtaagacgtgtcccctgcctttgcttgcccacagatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtacctcaacatgggtcttttggttttcatgagaaatctgctgctggctctgtgtctgttcctggtcctgggatttctgtactactctgcatggaagctccacctgctgcgctgggaggatagctctaaatatggacgcctgagccatagctcttttcctaagcaaagaccaagtgctgattctgtggtcttgtcatttgactctgttggacatactattggctctgaatatgacaaactgggttttctgcttaaccttgattctaaacttccccctgaattggcctcaaaatatgccaacttctctgagggagtgtgcaagcctggttatgcatctgccctgatgactgtgattttccctaaattctccaaacctgcccccatgttccttgatgactccttccggcgctgggcccgcattagagactttgtgcctccatttggcattaaagggcaggacaatctgataaaggcaatactgtctgctacaaaagattacagactcacaccagcactggacagcttgtcatgccgccgctgtatcattgttgggaatggtggtgttctggccaacaagagtttgggtcttaagattgatgactatgatgtggtcgttcgcctgaactctgcacctgtcaaaggctttgagaaagatgttggtggaaagacaacactgcggatcacttacccagagggggctattcagaagatggaacagtatgagaaagactccctgtttgtgctggcgggatttaaatggcaagactttaagtggctgaaatatattgtgtataaagaaaaggtctcagcttctgatggcttctggaaatcagtggctacccgggtgcctcgggagccacatgaaattcgcatactgaatccctatttcatccaagaagctgctttttcattcattggcctgccattcaataatggtctgatgggtcgggggaatatccccaccctgggttctgtggccatcacaatggctctgcataattgtgatgaggtggctgttgctggctttggatatgacatgagttcccctaatgctcccctgcattactatgagaacataaaaatgagtgccattaaggagtcatggactcataatatacaacgggagaaggaatttcttcgcaagctggttaaagccagagtgattacagatcttacatctgggatatgaggatc

SEQ ID NO: 3 - DNA, 9119 bp, Artificial Sequence
  pSIN-OM-1.8-GalT1-IRES-SialT3
cggatccccgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtacctcaacatgaaagaacctgcacttcctggtacttcactgcaaagagcatgtagactgctggtagcattttgcgccctgcacctgagcgcaaccctgctctactacctggctggatccagcctgactccaccccgctctccagaacctccccctcggaggccgcctccagccaacctctccctgccaccctcccggcctcctcctccccctgcggctcgcccccgcccaggacctgtttctgcacaaccccggaacctgccagattctgcaccatctggactgtgccccgatccaagtccactgctcgttggtcctctgcgggtggagtttagtcagccagtgaacctggaggaagtggcttctaccaatccggaggtcagggaaggagggagattcgccccaaaggactgcaaagcgctccagaaggtggctattattatccccttcaggaacagagaggagcacctgaagtattggctgtactacatgcacccgattcttcagagacagcaattggactatggggtctatgtgattaatcaagacggcgatgaagaatttaacagagctaaactgcttaatgtcggtttcactgaggcactcaaggaatacgattatgattgctttgtgttttccgatgtggatctgattcctatggacgaccgtaacacatataagtgctatagtcaaccacgtcacctgagtgtgtcaatggacaagtttggctttaggctgccgtataaccagtatttcggaggagtttcagcattgagtaaagaacagtttacaaaaatcaacgggttcccaaataactactgggggtggggcggagaggacgacgacatctacaacagactggtttttaaggggatggggatttcccgcccggatgcagtaataggcaagtgtcgtatgatacgccatagcagggatagaaagaacgaacccaaccctgagcgctttgaccggattgcacatacaagagaaactatgtcatctgatggacttaactctctttcatatgaggtgctgagaacagatcggttccccctgtacactagaatcacagtagatatcggggcacctgggtcataagcccgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataagcttgccacaaccatgggtcttttggttttcatgagaaatctgctgctggctctgtgtctgttcctggtcctgggatttctgtactactctgcatggaagctccacctgctgcgctgggaggatagctctaaatatggacgcctgagccatagctcttttcctaagcaaagaccaagtgctgattctgtggtcttgtcatttgactctgttggacatactattggctctgaatatgacaaactgggttttctgcttaaccttgattctaaacttccccctgaattggcctcaaaatatgccaacttctctgagggagtgtgcaagcctggttatgcatctgccctgatgactgtgattttccctaaattctccaaacctgcccccatgttccttgatgactccttccggcgctgggcccgcattagagactttgtgcctccatttggcattaaagggcaggacaatctgataaaggcaatactgtctgctacaaaagattacagactcacaccagcactggacagcttgtcatgccgccgctgtatcattgttgggaatggtggtgttctggccaacaagagtttgggtcttaagattgatgactatgatgtggtcgttcgcctgaactctgcacctgtcaaaggctttgagaaagatgttggtggaaagacaacactgcggatcacttacccagagggggctattcagaagatggaacagtatgagaaagactccctgtttgtgctggcgggatttaaatggcaagactttaagtggctgaaatatattgtgtataaagaaaaggtctcagcttctgatggcttctggaaatcagtggctacccgggtgcctcgggagccacatgaaattcgcatactgaatccctatttcatccaagaagctgctttttcattcattggcctgccattcaataatggtctgatgggtcgggggaatatccccaccctgggttctgtggccatcacaatggctctgcataattgtgatgaggtggctgttgctggctttggatatgacatgagttcccctaatgctcccctgcattactatgagaacataaaaatgagtgccattaaggagtcatggactcataatatacaacgggagaaggaatttcttcgcaagctggttaaagccagagtgattacagatcttacatctgggatatgaggatccggtac

SEQ ID NO: 4 - DNA, 2279 bp, Gallus gallus
gcatcccgtccccggcggcggcgcggggcgggcggcggtccccgtcagcgcggcggcatgaaggagccggcgctgcccggcacctcgctgcagcgggcctgccgcctcctcgtcgctttctgcgcgctgcacctctcggccacgctgctctactacctggcgggcagctccctgacgccgccgcgcagccccgagcctccgccgcgccgcccgcctcccgccaacctctcgctgccgccctcccgcccgccgccgccgcccgccgcccggccccgacccggaccggtctcggcacagccccgcaacctcccggactcggcgccgtcggggctgtgcccggacccgtccccgctgctcgtcggaccgctgcgcgtggagttctcccagcctgtgaacctggaggaggtggcgagcacaaaccctgaggtcagggagggaggtcgttttgctccaaaggactgcaaggcgctgcagaaagtagcaatcatcatcccgttccgaaaccgagaggagcatctgaagtactggctctattacatgcacccaattcttcaaaggcagcagctagattatggagtgtatgtcatcaaccaggatggagacgaagaatttaaccgtgctaaactgctgaatgtaggattcacggaagctttgaaggagtatgactatgactgctttgtgtttagtgatgtagacctgatcccaatggatgacaggaacacctacaagtgctacagccaaccaaggcacctttctgtctccatggataaattcggatttcggttaccctacaatcagtattttggaggtgtgtctgccttgagcaaagaacaattcacgaagatcaatgggtttccaaacaattactggggctggggaggcgaagatgatgacatctacaacaggctggtgttcaaaggcatgggcatatctcggccagatgctgtcattgggaaatgcagaatgattcgccactcgcgtgatcggaagaacgagcccaacccggagaggtttgaccgtattgctcacaccagggagacgatgagctctgatggcttgaactcgctctcctacgaggtgctaaggactgacaggttccctctgtacacgaggatcacagtggatatcggagcgcccggcagctgacacggccggcacggcggagacctcgggacggtgccccgcacgctgggctggcagattctttgtgtcgtcgggttttataagggttgtgatgaacaacacggaggtctctctgcatgtcagagcctctccaaaagggctggacgactgcttttcccgtcggttgtttttgtaactctgcctccagctctccattgttttgtaagttcagaggctgtacgtaacagttgtaaatacttcctttttgccaggagatgctgaatctgatccccgtgttcggtcaccgctggtcccgggttagtttgccaactgcagccgtggtgcaccagcagcgaccgcccatgatacggctttcttcttttttaattgggtggacgaaaacattccttttaattcattcctcgtttttatctctatgaaggactgtaaaacgctgctaaaattgtatgagtttactcatttcgttagattgttttttgttttttttttaagagaggcaaaattacgtggggtttcttcttcttttttttcttcctactggtgaccaaagcaaacaatcttctccgcgtgcagagcgcatgacgaataaccaagtgtggattcagcacacctcactattcctttcggtctcaaaagagacttccgagcgagctgaggcagatgtgccctcggagagctctgtgcgtgggctgggagccgcagggatgtgcagcagagctctccatgacccgcagcagctgctggctccccataacctgctgtcgggtgtggttttattttattttattttatcttttcttgcctgggcagagcaagacacctgggagatctcttcggtcggtcggtcagtttggtttgcttgtttgctcttcccccaaaagagcggatgggtttaattgcacaaggaattgatagccttaaaattcacagacacttttaccagtggtaggaagttgccacgctatttaaacatggtctgaaggttcttaagaacgacattctgcttgcaaggtcatgtgtgaaacttgaactcacttattacttttattgttgttgtaactttttgataacttttaaaagtaatttgtatatcctaagcggtatatttaataccagattaaagcagggtgcagcat

SEQ ID NO: 5 - protein, 362 aa, Gallus gallus
MKEPALPGTSLQRACRLLVAFCALHLSATLLYYLAGSSLTPPRSPEPPPRRPPPANLSLPPSRPPPPPAARPRPGPVSAQPRNLPDSAPSGLCPDPSPLLVGPLRVEFSQPVNLEEVASTNPEVREGGRFAPKDCKALQKVAIIIPFRNREEHLKYWLYYMHPILQRQQLDYGVYVINQDGDEEFNRAKLLNVGFTEALKEYDYDCFVFSDVDLIPMDDRNTYKCYSQPRHLSVSMDKFGFRLPYNQYFGGVSALSKEQFTKINGFPNNYWGWGGEDDDIYNRLVFKGMGISRPDAVIGKCRMIRHSRDRKNEPNPERFDRIAHTRETMSSDGLNSLSYEVLRTDRFPLYTRITVDIGAPGS

SEQ ID NO: 6 - DNA, 2420 bp, Gallus gallus
gtggcggtggcccggccggcagggcccccgcagccccggcatgggcgcccgcggccggcggccgggggaagcggcgggggccgctgacgcgccggggccgcgggaggaggtggtggcggcggtggcggcggcgggggacggtgcggggcggccgcggatcgggaccgtggctggagctgcctgcctccctgtgcccagaagatgaccaggttgctcttgggggtgaccctggaaaggatttgcaaggccgtgctgctgctctgcctgctccactttgtcatcatcatgattctctactttgacgtctacgcgcagcacctggacttcttcagccgcttcaatgccaggaacacctcgcgcgtgcaccccttctccaactcctctcggcccaacagcacggcccccagctacggcccacgtggcgctgagcccccctcccccagcgccaagcccaacaccaaccgctccgtcacagagaagcccttgcagccctgccaggagatgccctccggcttagtcgggcgcctgctcattgagttcagctcccctatgagcatggagcgggtgcaacgggagaaccctgacgtgagcctgggtggcaagtacacccccccagattgcctgccccggcagaaggtggccatcctcatccccttccggcaccgcgagcaccacctcaaatactggctgcactacctgcaccccatcctgcgccggcagaaggtggcttatggcatctacatcatcaaccagtatggcgaggacaccttcaaccgggccaagctgctcaatgtgggcttcctggaggcgctgaaggatgacgaggagtacgactgcttcattttcagcgatgtggacctcatccccatggatgaccgcaacctgtaccgctgctatgagcagccacggcactttgctgttggcatggacaagtttgggttcaggttgccctatgcagggtacttcggtggtgtctctgggctgagcaagtcccagttcctaaagatcaacggctttcccaacgagtactggggctggggaggagaggacgacgacatctttaaccggatctccctgaatggcatgaaggtgtcgaggcccgacatccgcatggggaggtatcgcatgatcaagcacgaacgtgacaaacacaacgagcccaacccgcagagattcaccaagatccagaacaccaaaatgaccatgaagcgggatgggatcagctcactgcagtaccggctggtggaggtgtcacgccagcccatgtacaccaacatcacggtggagattggcaggccgcccccacgcttggcccggggctagtgcttgccctgcaggcaaagctgcatgaggctggcgctctgtcgcagggctggctggacgctgtggatgttgccccagcccctgggcaaggactgaacggggatgttttctgcctactctgctgccttttggagacgctgtgccccagcctacctgttggtcctgaggatttctgcaatctgttgtccctcctttccccatccctacaagtgtgtttccagaacccccatactatgcgtgttggctgaagcacccgttcgccctgcgtgcagctcccagacagagggaggggacagtcccagccctggtgaggagccccttgcccacgtcacgtcccgcctgcaccctaggagggaaggatgagccccaaggtcagcctagcccccagtccccaccggtgctgcgagaagcgggatgcaggcttcccccttcaccagcgctggagctgctactaccctggctgaaggcatgggaggtagcccaggcccccacagcaggcaggatcggacagacagatgtggctcactgtcttcctctgctttagtctggtgctcagggctgggtctcagctctgctaaacagcgacctctggttagcaaacaccccttgctgtgcctcagtttccccgggctggcagccacgtcccctttcccctctctgaaggcagatgctgtgtgcgtgtccctgttaacccacacatgcaccagctctcccaactttgggcagtagggtgacgtgaaacctcacagcccctcttggccaggggtctgccccggggaatctccacccagatgctgtttgtaggcagtggggactggctgctctgcccttgctgctctccagcttcccctctctgctctggggcagggagaagaggaacagggccatgcggcaggtgccccatctctccccacttcccctccttggggctggggcacagccacccccctgcagccagctagaagagctgggcagcaggggcactggcaacttttgtacatttgaatgtctgaccctttttttgagcgtacgttgaatgcagcattcggtcatagagacctgggtttttgtatttaataaaaatttcaaaagttaac

SEQ ID NO: 7 - protein, 373 aa, Gallus gallus
MTRLLLGVTLERICKAVLLLCLLHFVIIMILYFDVYAQHLDFFSRFNARNTSRVHPFSNSSRPNSTAPSYGPRGAEPPSPSAKPNTNRSVTEKPLQPCQEMPSGLVGRLLIEFSSPMSMERVQRENPDVSLGGKYTPPDCLPRQKVAILIPFRHREHHLKYWLHYLHPILRRQKVAYGIYIINQYGEDTFNRAKLLNVGFLEALKDDEEYDCFIFSDVDLIPMDDRNLYRCYEQPRHFAVGMDKFGFRLPYAGYFGGVSGLSKSQFLKINGFPNEYWGWGGEDDDIFNRISLNGMKVSRPDIRMGRYRMIKHERDKHNEPNPQRFTKIQNTKMTMKRDGISSLQYRLVEVSRQPMYTNITVEIGRPPPRLARG

SEQ ID NO: 8 - DNA, 1830 bp, Gallus gallus
atgtccctgtcccgcgtggagaacccctgcttcctgctgttcctgctcgtcttccaagccgtgttcatcctgatactgtaccgaggtggagcctcgagcgtgttccggggctttttggagtcgcagcgcgttttggattactccaaaagccacgacgtgtacacgaacctcagcctgctggccccggctggcggcggggcggcgctgccctactgctcggagcgctcacccatcgccgttggtccattaaccatcacttttgacgtgctccctagtgaaagaacgatcatccaaaaaaatccttttgttcagtccggaggccactacagaccacctcactgcttggcccgctacaagtcagccatccttgtagcatacagtaaccaggagaaataccttcaccatcttctctactacattcatcctttcttgcagcgccagcagctcagctacagaatctacttgattcagcaggtggggaatggtacgtttaaccgagcaaagctgcttaatgttggtgtccgagaagccctgaaggatgaagactgggactgcctcctcctgcacgatgtgaacctggtacctgagaatgattataatctctatgtctgtgatgaatactatcccaaacatatggctagtgccatggataaatttcagtacaaccttccctacaagtccttttttggaggtgtatctgcattgactccagagcactacatgaagatgaatgggtttccaaacacatactggggcgacggtggtgaaacagatgacattgctgcaaggatccagttagcaggcatgagaattgtccggaccccaccacaccttggacgctacaaagtgatggactacaacagagagacagaagagccttggagaaggcctgcttcccaccacaacactggaaaaacttggaaggatgatgggatgaactctttagagttcaagctcctttccagaacaaagcatcctctttataccaacgtcactgtggacattggatatgttccccccttttcttaagataatgaaaactgaaacgtggtgttggaattcactgtggcagcacagttatgggtactcagcctctacctctggctgcggtgcagtctgcagcacctgagactaatcctggtgtctttcatacattgaacttttcttcggattataggaggctttgaagaaaaggcttcaggagtgagacatgatagctacaaacaggagctggcttactgtagaagtcctttaaagcactgtaaaactgagccaaatctacatgtcatgcctcaggctggatagagactgtctccttgacagtaagttgacccaagttttctggaacctttgttccgtaacgggatggctctgccctgctccttactcaactggtaagtgggattcaggctgtcctgtcagtcctctcaaatgctgtattttgagaaagatcttacttggtgttgaagcctattacagttctgtaaatactctttgtgaatttgtgtcaagaaagttgaggtgtgtttgtttggattaagatttttctagagtatttaataagactttaataaaggagaaagttgccctgaagttggttgaaactagagcttaaaaattcctggttgtctggccacattatgaatgagtgtgtgagttcttactgtagccataagtaattcacatctaaagaggcctcagactgtcagctatgctcagcagatggaaattaatctaccatttcagccattgctcaaatgttaattatctctcaaggttgatctgtattcaattaaaacacttctgagaaatgaaatactagaggaaaaa

SEQ ID NO: 9 - protein, 342 aa, Gallus gallus
MSLSRVENPCFLLFLLVFQAVFILILYRGGASSVFRGFLESQRVLDYSKSHDVYTNLSLLAPAGGGAALPYCSERSPIAVGPLTITFDVLPSERTIIQKNPFVQSGGHYRPPHCLARYKSAILVAYSNQEKYLHHLLYYIHPFLQRQQLSYRIYLIQQVGNGTFNRAKLLNVGVREALKDEDWDCLLLHDVNLVPENDYNLYVCDEYYPKHMASAMDKFQYNLPYKSFFGGVSALTPEHYMKMNGFPNTYWGDGGETDDIAARIQLAGMRIVRTPPHLGRYKVMDYNRETEEPWRRPASHHNTGKTWKDDGMNSLEFKLLSRTKHPLYTNVTVDIGYVPPFS

SEQ ID NO: 10 - DNA, 1836 bp, Gallus gallus
ggggcaccggcggcagagaggcccggggaggttaagtcatgttttctgacactcgaagaacagtcagcgctgcaattccatgagtcacagaaccacaagaagtgcggctgccctccctacatgcagcttcccttctttactgtaacgttcttcttggcacctctggcttcttccctactgagtgcacagcgggggacccacggtggcatcggggggagcgatggccataagcttgtatgtatttcacttcttcaataagttcaaagtgttccttcttgtcaccttgtgtttgatgatgctatgggctgcgttcagttactttgtggattctggacagacaattcctaaacttaagagtgtgggggagcattttggaaagataatcagcttggagaagaaagaggacagtcagaaggaagaaaagatgaagataactgaaggagttcctgcaacaaagccacctcagggtccctgtccagctctgtctccgtacctgcgaggtgccagcaaactgaccttcagtccatctctcacgctagaagaagtggaaaaggagaaccctcaggtggccaagggccgataccaccctgcagagtgttcagccttgcagcgtgtggccatcctcatcccgcaccgcaaccgtgagaggcatctgctgtacctcctggagcacctgcacccgttcctgcagaggcagcagctggaatatggcatctacgttatccaccaggctggcagcaccaaatttaatcgagctaaactgctgaacgtgggatacttagaggccctaaaagaagagaactgggactgtttcattttccatgacgtggatctggtgccagagaatgacttcaatatttacatgtgtgacagacaacccaagcaccttgtagttggccggaacagtactggatacaggttacgttaccagggatattttggaggcgtaacagctctaacaagagaccagttttccatggtgaatggattctctaacaactattggggttggggcggagaagatgacgaccttcgaatcagggttgagatgcagaagatgcgagtgatgaggccatctgctgatgtagccagatacacaatgatcttccacaaccgtgaccatggcaatgaggagaacagagagaggatgaagcttctgcgtcaggtatctagaacatggaaaacagatgggttgaattcctgttcctatagactgctgtcagtggaacataaccctttatacatcaacatcacggtagatttcagcatgcagccaaagatctcataggggtgagcaccgcacaggttttaggaaatggcagcagatcctgtttgtggttggtggcacagcagatgacttgcagcgctctgcttgaaagagtactttagcagcacagaagaatgtttttctgcatggcatctaattagtcagcagagaagctcattttctgaggactggagaggcggactgacccctgggcaggtcctggtgttcttctatgcactttctgctgagatgttcgcaagtttccttgtttgggctggtccagtagaaggacttgaatcaagtctcctgaaagaaatatttcttaaaactattcctgaacttccagtttgaagtaggaaggtgacactgccaaggcttcaaaaggagcaggccagtcttttcccctcaaaagcacaagaattttttaccatctataaacttgttgagaaaagcttgtttcttctagcctgaagaaaaactgcttgtggggtggaaacagcaataatggtaataggggagaaatgattaaaaaaatctctcaataaaatataatctgcttaattcaaaaa

SEQ ID NO: 11 - protein, 355 aa, Gallus gallus
MAISLYVFHFFNKFKVFLLVTLCLMMLWAAFSYFVDSGQTIPKLKSVGEHFGKIISLEKKEDSQKEEKMKITEGVPATKPPQGPCPALSPYLRGASKLTFSPSLTLEEVEKENPQVAKGRYHPAECSALQRVAILIPHRNRERHLLYLLEHLHPFLQRQQLEYGIYVIHQAGSTKFNRAKLLNVGYLEALKEENWDCFIFHDVDLVPENDFNIYMCDRQPKHLVVGRNSTGYRLRYQGYFGGVTALTRDQFSMVNGFSNNYWGWGGEDDDLRIRVEMQKMRVMRPSADVARYTMIFHNRDHGNEENRERMKLLRQVSRTWKTDGLNSCSYRLLSVEHNPLYINITVDFSMQPKIS

SEQ ID NO: 12 - DNA, 1773 bp, Gallus gallus
atgcccatgtgttcaaaggctacctcagattttctggagccatcactgtgtgtcccagaacaatgcctggatatcctgaggccatccctatatgccccaggaccatgtccaggcgtcctaagactatcaccagttgtcccatggtcatcactggtgcttggatgccatcatcgtttgtcccagaaccccactaaggtgccccaaggccattactgcctgcctcaaggccagctctggcattctgaggccaccgctcagcgtccccagcatcgtgcccgggtgtcgtggggctcccatcgactgtcacagggccacctctgtttccctggggaagctattggtgagaacagagcgaggggcgggcggcgcacgtcatcctttgccgaagaacactcccagcagcatgctctgcagcgggggccggagtttgcagccggttctcgctccgcctcgccccgccccgccccgccccgcgtcgctgtggctccttcatgcggcggcgttgggcgcgggcggtggctcggagcggctgcggtgcagcgcttcccgccgggcctcggcgcatgtgagcggcagggcggcggcacatcggccatgcggtggccccgcggcccccgcggcgcctggcggctgttgccccggcgctcgctgctggccgtgctcttcctcttctcgctctcctcctccttcctctacttcgtatatgtggcgccgggcatcgtgaacacctacctcttcatgatgcaagcccaaggcatcatgattcgtgaaaacatgagaacaataggagctcaggtgtatgaacaggtggtccgcagtgcctatgccaagaggaacagcagtgtgaatgactcagattatcctcttgacttgaatcacaatgaaacctttctgcaagctacaacttttcttcctgaagattttacgtacttccccaaccacacctgtcctgagaggctcccttctatgaagggccccattgatgtaaatatgagcgagattacgatggaggacatccaccagttcttctccagagacccttccatcaagctgggaggccactggaagccgagcgactgcctgcctcgctggaaggtggcgatcctgatcccattccgcaatcgctatgaacatcttccagtccttttcaggcaccttattccaatgctgcagcgtcagcgtttacagtttgcattttatgttgtggaacaagctggtactcagcccttcaaccgtgccatgctcttcaatgttggctttcgggaagcgatgaaggacttggactgggactgtctcatcttccatgatgtggaccacataccagaaaatgaccgcaactattatgggtgtggacagatgccgagacactttgcggccaagctggacaagtacatgtacctgttgccctataatgaattcttcggtggagtgagcggcctgactgttgagcagttctggaagattaatggtttcccaaatgccttctggggctggggcggtgaggatgacgacttatggaacagagtgcagtatgcaggctattcagtgactcgaccagaaggagacacaggaaaatacaaatcaattccccaccatcatcgaggagaagtgcagttcctaggaaggtatgccttgctgaggaagtcaaaagaaaggcaagccctggatggcctcaataatttgaactactttcctaatgtcacatatgacgccttgtataagaacatcactgttaacctgacaccagagctggctctggtaactgaatattaa

SEQ ID NO: 13 - protein, 392 aa, Gallus gallus
MRWPRGPRGAWRLLPRRSLLAVLFLFSLSSSFLYFVYVAPGIVNTYLFMMQAQGIMIRENMRTIGAQVYEQVVRSAYAKRNSSVNDSDYPLDLNHNETFLQATTFLPEDFTYFPNHTCPERLPSMKGPIDVNMSEITMEDIHQFFSRDPSIKLGGHWKPSDCLPRWKVAILIPFRNRYEHLPVLFRHLIPMLQRQRLQFAFYVVEQAGTQPFNRAMLFNVGFREAMKDLDWDCLIFHDVDHIPENDRNYYGCGQMPRHFAAKLDKYMYLLPYNEFFGGVSGLTVEQFWKINGFPNAFWGWGGEDDDLWNRVQYAGYSVTRPEGDTGKYKSIPHHHRGEVQFLGRYALLRKSKERQALDGLNNLNYFPNVTYDALYKNITVNLTPELALVTEY

SEQ ID NO: 14 - DNA, 4819 bp, Gallus gallus
gagagcgggcgcccagcggcgggagcggcccccgagccccgcggcagcgctaggggacccttcccgctcaggccgccgcctcctcggctctacgggccctccgccgggcggtgggagcggcgggagcgaagcacagccctcgcccccgtcccgccgccgctccatgggaggagcccgccgccgccctgcgggaagcgcggccgccgcgcgccgggattgaggggagcgggcagctctgagacggcgggaggatgcccctgttccggaaggtgctgcgcgtctccaatcgctccatgctcgccttcatcttcttcttctccttctcctcctcctgcctctacttcatctacgtggcccccggcatagcaaatacatatctcttcatggtgcaagcacgtggtataatgttgagagaaaatgtaaaaacaataggacacatgatcagattgtatactaacaaaaatacaacactgaatggaacagattatcctgaaggaaacaattctagtgactgtgttgctcaaacaacaatgtatcttccagaaaacttcacttactctccttaccaggcttgtccggagaaactgccttacatgagaggccttattgatgtaaatatgagtgaaattagttttgatgaaattcagcaactattttcaaaagacttggacattaaaccaggaggacactggaaacccaaagactgtaagccacgatggaaggtggcgatcatcattccttttcgtaatcgtcacgagcatcttccaattttcttccggcatctgataccgatgttgcagaagcagcggctggaatttgccttctatgttgttgaacagacaggtacacaaccttttaatcgtgcaatgctttttaacgttggcttcaaggaggccatgaaggatgttgtctgggactgcataatatttcatgatgtggatcacttacctgaaaatgaccgaaattattacggatgtggagaaatgccacgtcattttgcagcaaagttggacaaatacatgtacattcttccatacaatgagttctttggtggtgtaagtggactgacagtggaacaattcaagaagattaatggatttccaaatgccttctggggttggggtggagaagatgatgatctttggaacagggttcactatgctggatacaacgtaacaagaccagagggagacttagggaaatacaaatccattcctcatcatcacagaggtgaagtccagtttttaggaagatataaacttctgaggtattccagagaacgtcagtatattgatgggttgaacaatttagtatatactcctaaaatacttgtcagtagattgtataaaaatgtaactgttaatcttatgccagaacttgctcctattagagactattgatggaaatggtgtgacaagctatcctactggagtaaacttttaatgcactggaaggtattaatagacactgaagacgctgtaaaacaaaacaaaaaatcacaacaatcaacgtcttagaattagggatctttgtccatttgatgatgcatatttgggatgagaagtaaagtgattgtatgtgccgtgcattctgttcagaaagaaaagccagcagctaccactcagatgtttacagcatgagactactgttcaagccttcgttctccatgttctctatcttaaccactcagctaaataaactgcagaaaacagacttgctagcttcactggaagtagaggcattcttcttccaggactttttttatactaaattgctcccactctgccctcctgtatttaaatgaatgcttttgttcctttttaaaatgtgttttgtaaatatgtgatgtaaattaatgtgtgtacattgcttttaaattgctcaatattttatgcttcagtatgtatttgggtgtgttcttgtttgaattctataggaatgtttttattagcatgaaagaacaagtataagatgcaagtatccttaaaaaaggttataccttatgtgagatgaaggaaatactaattgttggccagctatgactgtaaactgttatactagttttgagctctaggcctcctgcatatctatatagaagaatcaatttcatatatgaactttctccaaaagaaagcttctaattttatttattgccagcaaaattatacaataccctgcctgccatctaaatcatatttatatacctattgcatgtgtattatggaaaatcttgcagttgttacatactatgatctacaggaactcttaaatgtttccacgtgtgccactagtgtcaatgtcagggattttaatgctaaaacaatgtgtccaggtgcgtacagtattttggtattgtcttttttttttaaatactattgagaagcacttttattcctccaaaatcagaagagccaaaaatgtgtcttcattggaagaatattaaagttagatttttaagaaaataataaaacatagttctaatgcttgcagtgtggggttttcagaaacatggtgtgggtcaaactgttctacttacgattgcataagtggaactgaaattaaactaagtgctttttaaaattccaactataatatattaatcaatatagctattgaagggcctattcgagtacagtgctgaatgcttttatgatgcactaagtatcctcaagtctatgacttcaaatgagattgaggttgcacagttccactaagaattggtcctgtctgtttctctatttttgtggtgtaaaatgtatgagtaactagcctctctccttggcttagaatgaaaaacatatcttcttattttcctattatctgtttgtttgtaatgttaagctacttataagtaactcaatgctaagaagtattttttcctttttttttttttacattctttgtttaacactaaggagaaggactgaaacatttattttctatgttaatgcaaatatattgactaaactctcctgaactgttttgtttgtcgcagttattcaactctttaggaaccaaagactgacttctgcttctgtaaagaatggaaagtatccgtagagtgttctgtaagaatgtagacaaaaaatactaactcgttactatgtgttggtttcctgaattactgccacaaatagtgtggtgctatgtatattttgtttgccataaacactttttatttccttgatgatcccagcagcaaattttgctcttagtcattcttaggtaaagttaggattttacttgtagacatcccaaatattttgtagaaatgtaatgatttagttgtagtactcaagagctaagggataaaccttatgggaattgccctaaaatctaccagtttatctaatatgcattagcaatgatgtagtgcaatgaaaataatgcaaagtataaagtgaacagaacatcttttcaaaagttagatcccctcctgatatatttttataactgagtgtataggctattctgaagcatatggataaagggagataatatatgtatgatcaacaacatctttaaataaaattggtcttatttcataacagactaatacaattctttaagaactgctgaataaattttaaattgttgtaattttgatgtgatttctggttttcagtacaaagttcaactacaattcaacacctatgattaccaaacatcctttggtactgtacgtctgtactgttttctgaagcgtgcttttgtgcccatttggtaggcattctgtgaaatgtgcaattttttaaccattcaaatgcatatttatgcatagatacatatatacataatatattacacacttgctgtggctgaactaatataactttatagctgtaagtgattaatcatgtttggtcttaggaaaaatattattttaaaccaggatgtaaatggattatgacatatagcggagttcttgaaaggtttgcatttttggtgcccaaaagtgatggacgttaccttatgaaatgttaaccaacactcctctctaaataactttgatcactttgttgagaagttttcaaaggtgccatcttaaattacatccaaataatgtttgtatgctatctgcttgagctcagtactattctgtactgaattttaacctaaacagtgccttgaaacaaaataaatgcgatgcagaactaagagttctgtacattccttttgatatttttcataattcttgtaaaacatggggagaggccaagcacattgttttttgcattggtgatgaggcaaataaagaacatcttctctttgaggagttaacttggagggagaagaaaggaattatttttacgagattctgaagatcttgcaattgagagtaaaaattttacagaaatattaacagtagaatctgcttcatatgctgagatactcttaaacccattttccgcaatataaaactaaatagaagaatataaaatataaaatccatttcaaaatagaaaatatattgaaaaatgtatgaaatttcctttttcttggatcttgccagactatttttctattacgtttactaatgactgtgttgaagtggagttctgatgagccagttacttaaaaatattacaagcacccactgcatatcagcaagaggaagtgtacttaagattatttagtttgtagaggtatgtaagataggccttaatgaaaaatgcactgaaacatatgctgggaatttcttgtgtactatcataacttttgttttttttgttgttgttgttcatgagaatgtttgtacttttttatcattgtcttttatgaaatataatgttctaaagctggaaaaaaaaaaaaaaaa

SEQ ID NO: 15 - protein, 368 aa, Gallus gallus
MLAFIFFFSFSSSCLYFIYVAPGIANTYLFMVQARGIMLRENVKTIGHMIRLYTNKNTTLNGTDYPEGNNSSDCVAQTTMYLPENFTYSPYQACPEKLPYMRGLIDVNMSEISFDEIQQLFSKDLDIKPGGHWKPKDCKPRWKVAIIIPFRNRHEHLPIFFRHLIPMLQKQRLEFAFYVVEQTGTQPFNRAMLFNVGFKEAMKDVVWDCIIFHDVDHLPENDRNYYGCGEMPRHFAAKLDKYMYILPYNEFFGGVSGLTVEQFKKINGFPNAFWGWGGEDDDLWNRVHYAGYNVTRPEGDLGKYKSIPHHHRGEVQFLGRYKLLRYSRERQYIDGLNNLVYTPKILVSRLYKNVTVNLMPELAPIRDY

SEQ ID NO: 16 - DNA, 1319 bp, Gallus gallus
ggaacggcgcggctcggcccggccagcgtgtcccggcggcggggccggggtccgccatggggccgggccgccggagagccgcgctgcgcctgcggggcggaggctccccgcagctcctgggtctcctggccggcaagttctccatcttccagctattcttcctcgcgctgctgctgggcttcgcctcgctgctctggctgcagctcagctgctcgggcgaagcgccctcccccgggcgcggggccccccggccgccctgcccgcccgaaccccccgccccgccggccgacgacccttcgtgggggccgcaccgcctggccctgctcgtgcccttccgcgagcgcttcgaggagctgctggccttcgtgccctacatgcaccgcttcctcagcaagaagaggatccgccaccacatcctggtgctcaaccaggtggaccacttcaggtttaacagagcgtcgctgatcaacgtgggcttcctggagagcggcaacgacacggactacatcgccatgcacgacgtcgacctgctgcccctcaacgagcagctggactacggcttccccgaggccgggcccttccacgtggcgtccccagagctgcacccgctgtaccactacaaaacctacgtgggagggatcctgctgctcaccaagcagcattatgagatgtgcaatggcatgtccaaccgcttctggggctggggacgggaggacgatgagttttatcgacgcatcaaaggagctggcctccaggttcatcgtccctctggaatcacaactgggtatgagactttccagcacctgcatgacccagcctggaggaagagggaccagaagcgcattgctgcgcagaagcaggagcagtttaaggtggatcgggagggaggtctgaacaacgtgagataccggattgagtcacggactgctctgagcgtggcaggggccccctgcactgtccttaacatcttgttggactgcgacatgagtgagacaccgtggtgcacgtttggctgagctgtgtcccatgtgccagcatgcgctgcgctcatgccaaggcgccagggctgcgccgagctgcttggagcaaggcagagttttgcagcaggccagcacggtgctgctggcaggacccagaggagcagaaagggctgagtgcttgaatttgctgggatcagcagaagaggccaagagcaggactccatggcattgctgtgagtgatgcggctgtctcctagggcaggtgcaggaggcgtttttcccatgctgggtatggccgagctgccacccagttcagaggacaataaagaactatcaagg

SEQ ID NO: 17 - protein, 319 aa, Gallus gallus
MGPGRRRAALRLRGGGSPQLLGLLAGKFSIFQLFFLALLLGFASLLWLQLSCSGEAPSPGRGAPRPPCPPEPPAPPADDPSWGPHRLALLVPFRERFEELLAFVPYMHRFLSKKRIRHHILVLNQVDHFRFNRASLINVGFLESGNDTDYIAMHDVDLLPLNEQLDYGFPEAGPFHVASPELHPLYHYKTYVGGILLLTKQHYEMCNGMSNRFWGWGREDDEFYRRIKGAGLQVHRPSGITTGYETFQHLHDPAWRKRDQKRIAAQKQEQFKVDREGGLNNVRYRIESRTALSVAGAPCTVLNILLDCDMSETPWCTFG

SEQ ID NO: 18 - DNA, 1838 bp, Gallus gallus
cccatggacagaggtactgcaggcagtaggtgcagcctaggccccaggtggtgaccggccccaagaaggccggcagcagcctgtccttgtggccttagcttccccccacgtcgtgctccaccagcagcaagatggtcaccgtcaggaaaaggaacgtgaaggtcttcacattcgccttcgtactcatcacggtgacgtcattcctgctgaactacaagcaccaggtgaccatgaccacttgggatcctaaacatatcatcagtcagttttctgagcaagtccgaaaactcatcaaatttcctaggaggccgtgcagctgtagcacctgtatttccgagctgggacattccctctggttcgaccagaggtttaactcaactatgcaacctttcctgacctcacaaaatgccttgatcccagaggacagctacaggtggtggctgaaactgcaaggagagaaatctccaaagaatattaatgatactctcaaggaattgtttgggatcattcctggggacagggacccactgcaggagcgaggcactttctcatgcagacggtgtgccgtcgttggcaactccggcaaccttcgtcagtctcaatatggccaagatattgactcccatgactttgtgctcagaatgaaccgtgcacccaccattggctacgaatcagatgttgggagcaagactacccaccactttgtttatccagagagctacaaagagctggcagaaaatgtgagcatgatcgtgatccccttcaaaaccctggacctgcgctggattgttaccgctctcaccacaggcactatcaacttcacatatgttcctgttccacggaaaatcaaagtcagaaaagaaaaggtcctgatttacaatccatcctttatcaaatacgtctatgaaaactggcttcagaatcatggaagatacccttccacaggccttctttctgtgatatttgcactccatgtatgcgatgaggtgaatgtgtatggttttggagcagacagcaaaggacactggcatcactactgggaaaataatgcttcagctggggctttccgacagacaggtgtccatgatggagattttgagttcaatgtaactttgactcttgcctccattgaaaaaataaaatttttcaagggcagatgaccctagccacagggacaagcaggggctgcaatttccaacatgcagcagcacaaagctcagtgaagatgatctggatgacagccaggtttgaaggtgtgaatctggagtggattcggagtgtcacactgctgcagtgctcaccacagggagctcagctgaggaacagattcaatgctgcacttgatttgctatctatagatagctgggaactaccgatcagctgttggaataaatgacatcctgactcactccttagtctctacggattgattcctgaattactggtgaaagatctacctgttgcaattcagggaaacctagacacaggaactgctgtttgtatgtccattctttccttgagtaaggcaagaaatccttgaagaacatggaaaatgtcttctgggatttgatacctactagagtagctctgaacatcatagtaaggattacctcaaagaaattaattcagttctgctgttaatcttctttttgaattctcttctgtttcctctattacttctggtttcatgcactataaatcaaaaacgtgatgaagttgccggagtagtaactgtttctaccattgccacattctgcattgatggcatctagaaaatacagatcaattcacctgtgatcattacttatttattattgcttcctatacagccatagtaa

SEQ ID NO: 19 - protein, 342 aa, Gallus gallus
MVTVRKRNVKVFTFAFVLITVTSFLLNYKHQVTMTTWDPKHIISQFSEQVRKLIKFPRRPCSCSTCISELGHSLWFDQRFNSTMQPFLTSQNALIPEDSYRWWLKLQGEKSPKNINDTLKELFGIIPGDRDPLQERGTFSCRRCAVVGNSGNLRQSQYGQDIDSHDFVLRMNRAPTIGYESDVGSKTTHHFVYPESYKELAENVSMIVIPFKTLDLRWIVTALTTGTINFTYVPVPRKIKVRKEKVLIYNPSFIKYVYENWLQNHGRYPSTGLLSVIFALHVCDEVNVYGFGADSKGHWHHYWENNASAGAFRQTGVHDGDFEFNVTLTLASIEKIKFFKGR

SEQ ID NO: 20 - DNA, 1652 bp, Gallus gallus
ggggcccggaggctcccggcggtggggccgggccggagcggagcgaggctgttgccaccgacgtgtccctggcacaggacgatgagtgccaccgcggcccgggaccggctggggacggctccccgagcccacggggccaccgggacgcggagctacggctgagcccggcgtgccgagccgcgccgagccaggcggatggaaagctgcagcccggcagggagccccgcgcccaccccgcgccccagccctatgcccggggagcggcgccgagcctgcccgcccgcccaccatgaagtgctcgctgcgcgtctgcttcctctcgaccgccttcctcctcatcttcgtcatgtcggtgctcttcacctactcccaccacagcatcgcctacctggaccccggcgggctgggcggcatccaccgggtgaagctggtgcccggttacgccggcgtgcggcggctgagccacggcgtgccgtaccccaggggctgtgcgtgccgccgctgccccgaggatgccgccgccgccgccgccgcctggtttgacagccgctatgacggcggtgtgtctccggtgtggaccaaggagaacatggagctgccgcccgacgtgcagcggtggtggatgatgctgcagccccagttcaagtcccacaacacgcaggaggtgctgagcaagctcttccagatcgtgccaggggagaacccttaccgctggcgcgacccgcgtcactgccggcgctgcgccgtggtcggcaactcgggcaacctgcgtggttccggctatgggcacgagatcgatgggcacgacttcatcatgaggatgaaccaggcacccacggtgggcttcgagggggacgtgggcagccggaccacgcaccacttcatgtaccccgagagtgccaagaacctgcctgccaacgtcagctttgtgctggtgcccttcaaaaccttggacctgctctggatcgccagcgccctctccactggccagatcaggttcacctacgcgcccgtgaagcctttcctgcgggtggacaaagagaaggtgcaaatctacaaccctgccttcttcaagtacatccacgaccgctggacggagcaccacgggcgctacccctccaccggcatgctggtgctcttcttcgccctccacgtctgtgatgaggtgaacgtcttcgggtttggcgccgacagccggggcaattggcaccactattgggagaacaaccgctacgccggcgagttccgcaagaccggggtgcacgacgccgacttcgaggcgcacatcatcgacatgctggccaaaaccagcaggattgagggtgtaccggggcaataactgagggccgggcggccgcgcgtcctccagctcctaaccccggcactgccagagctgccccggctgccgttgggtgtcggcggccgatgggggttctgagtactcggcagactttgtgtggttgggggagttctgacttgaccttgttagtattaaggaacccgcttcagccaagtgaggattttgtagacgccgcagcgccccagccggccgggggatgcgcccaactcgtatctgttacagtcaaaccaaatggctgctcttttttaaaaaccagaacaagcaaaaaaccgtacaaaaagccccctaaaaa

SEQ ID NO: 21 - protein, 349 aa, Gallus gallus
MKCSLRVCFLSTAFLLIFVMSVLFTYSHHSIAYLDPGGLGGIHRVKLVPGYAGVRRLSHGVPYPRGCACRRCPEDAAAAAAAWFDSRYDGGVSPVWTKENMELPPDVQRWWMMLQPQFKSHNTQEVLSKLFQIVPGENPYRWRDPRHCRRCAVVGNSGNLRGSGYGHEIDGHDFIMRMNQAPTVGFEGDVGSRTTHHFMYPESAKNLPANVSFVLVPFKTLDLLWIASALSTGQIRFTYAPVKPFLRVDKEKVQIYNPAFFKYIHDRWTEHHGRYPSTGMLVLFFALHVCDEVNVFGFGADSRGNWHHYWENNRYAGEFRKTGVHDADFEAHIIDMLAKTSRIEGVPGQ

SEQ ID NO: 22 - DNA, 2462 bp, Gallus gallus
atgggactgctggtgttcatgcgcaacctgctgctcgccctctgcctgttcctggtgctgggctttctgtactactccgcctggaagctgcacctcctccgctgggaggactccagtaagtacgggcgcctttcccattcctcgttcccgaaacaaagacccagtgctgattcagtggttctttcctttgactccgttggacatacaataggctcagagtatgacaaactgggctttctcctgaacctggactcaaaattgcctccagaattggcatctaagtatgcaaatttctctgagggagtgtgcaagcctggttatgcatcagcactcatgactgtcatcttcccaaagttctcgaagccggcaccaatgttcttagatgattccttcaggagatgggctcgtatcagggactttgtacccccctttggaattaaaggacaagataatttgatcaaagccatcctgtcagcaaccaaagattaccgtctaactccagctcttgacagtctcagctgccgacgatgtattattgtgggaaatggtggagtacttgccaataagtcattggggctaaagattgatgactatgatgttgttgtcaggctgaactcggctccggtgaaggggtttgaaaaagatgtgggtggcaaaacaactctccgcatcacatatcctgaaggtgcaatccagaagatggagcagtatgagaaggattccctgtttgtcctggcgggattcaaatggcaggatttcaagtggctgaagtacattgtttacaaggagaaagtgagcgcctctgatggcttttggaagtcagtggcaacccgtgtccccagagaacctcatgagatacgtatcctgaatccctacttcatccaggaggcagctttcagcttcattggactgcctttcaacaacggcctgatgggcagagggaacatccccaccttgggaagcgtagcaataaccatggcactccacaactgtgatgaggtggcggtcgctgggtttggctatgacatgagttcacccaatgcgccactgcactactatgagaacatcaagatgtctgccatcaaagagtcctggacgcacaacatccaacgggagaaggagttcctgcggaagctggtgaaagcccgagtcatcacggacctgaccagtgggatctgagtggctgcagcccctgcctcaaagggaggagaggaagacatacgctgcggctctgggagcaggcactggcagccccccacaagaatcccacttccctggagacacacagagatgcccgggtgctctgggaaggccctctcgcatcgccgggctgcaggaaggttgcatctgctgcctccagtcctggagctggcaggaggcgggcgaggggctcagtgggcagttcttgaactctgcatcacagacggatcttctgtgtccagaattaaacaggaaagactcaggagagagaagaaaggtttgtgaataaagcgatttgtgccaaatgggaggtgacgctgccccgaggcagcagtgcctgaatgtacaaagtagtattttttaaaagaaactctgctggaatcatcgtagaattaccaacgtgcaaagcaagtctgcttgtgcacagccctgcagaaagctcggcctcaccacgtcccatctgcattctcactgccctcagacctttccccaggaaaacaaatccgtccaaaccgtcagtgttgttggtgctgctataatttaaagggaggttggcttcccctccttctccacttggagcttctcattggagatgagcacgggtttgtttttctgcacttttcctcccttcctgcatagaagcggcggcggcagccactcacttggctgtgttttccatagctgtttgctctgccctgacgctggaactggtggctctctgcctgcagcaggcccaccgtgccgcctgtcacagcgctgcggagcccacctcgtcgtgctcagggctgtcaggtccgtgcttgctgtgcagagccctcgtggagtccgtgcagatcggtgtcaccacttctggacagcatcctgctttgtttttgtgggggagatcagtggtttgttttttggaaggaggtccgatgctgcgtggggatctgaagctttgcatattgaagaccaggccaccgaagatgttttatgttccggactcgatcatgttccctatttaagtgacttgtgacctcagcaatgatggagcgtgctggcaagtgtgggggcctgctgggaaccctcgccttctgcttggcccctgctcatttcatgtccaagctcctcccgtgctgctcgagcgctgctgctcctcctgctctcaggagcacgtcccttcctttgctgtcttggtcccgagatgcagtatttgcacatttgatttgtgtacgtatttcagaggagctggaataaactgagcgccgtggctgagtgcaagg

SEQ ID NO: 23 - protein, 392 aa, Gallus gallus
MGLLVFMRNLLLALCLFLVLGFLYYSAWKLHLLRWEDSSKYGRLSHSSFPKQRPSADSVVLSFDSVGHTIGSEYDKLGFLLNLDSKLPPELASKYANFSEGVCKPGYASALMTVIFPKFSKPAPMFLDDSFRRWARIRDFVPPFGIKGQDNLIKAILSATKDYRLTPALDSLSCRRCIIVGNGGVLANKSLGLKIDDYDVVVRLNSAPVKGFEKDVGGKTTLRITYPEGAIQKMEQYEKDSLFVLAGFKWQDFKWLKYIVYKEKVSASDGFWKSVATRVPREPHEIRILNPYFIQEAAFSFIGLPFNNGLMGRGNIPTLGSVAITMALHNCDEVAVAGFGYDMSSPNAPLHYYENIKMSAIKESWTHNIQREKEFLRKLVKARVITDLTSGI

SEQ ID NO: 24 - protein, 374 aa, Gallus gallus
MGLLVFMRNLLLALCLFLVLGFLYYSAWKLHLLRWEDSNSVVLSFDSVGHTIGSEYDKLGFLLNLDSKLPPELASKYANFSEGVCKPGYASALMTVIFPKFSKPAPMFLDDSFRRWARIRDFVPPFGIKGQDNLIKAILSATKDYRLTPALDSLSCRRCIIVGNGGVLANKSLGLKIDDYDVVVRLNSAPVKGFEKDVGGKTTLRITYPEGAIQKMEQYEKDSLFVLAGFKWQDFKWLKYIVYKEKVSASDGFWKSVATRVPREPHEIRILNPYFIQEAAFSFIGLPFNNGLMGRGNIPTLGSVAITMALHNCDEVAVAGFGYDMSSPNAPLHYYENIKMSAIKESWTHNIQREKEFLRKLVKARVITDLTSGI

SEQ ID NO: 25 - DNA, 1622 bp, Gallus gallus
agagagcagaaggggtgctgctgtgcgcggacacttttctctgggaggaattggactttttcttctccaaactgtggggaggacatggaaattgggaagtctggggacagcgaagggggcagagagcccctgagcacatggtgagcagtgctgggccccgagatcccccagcctcacactgaggctctccctttttccttcaccagaagaattgaggatggccccatagccccgcagcccccggagctgctgccccctctgctcgcaccacgtggaagaactgtgagcggcagtggctgcgatgagagctccggccctcggctgatgcctgccctgctgataaagatgatcaataagtcccgagggaagatactgggagtgctggcgctgtttctggtcatggtgtggtactcgatataccgggaggacagcttttattttcctgtgcaagaaaacaagaccgtatgtcccattggggaggtggagaggaaggcagcacagctcatcgggaactacacgagggaccgcccgctcttcctgcagctgaaggattacttctgggtgaggacgccgtcgctctatgagctgccctacggcatcaaaggcagcgaggatgtcctcctgcgcctgctgtcggtcaccagttactcactgcctgagagcatccagagcctgaagtgtcggaggtgcgtggtggtgggcaacgggcaccggctccgcaacagctccatgggggacaccatcaacacctacgacgtggtgatcaggctgaacaacgcgccggtgcacggctatgagcaggacgtgggctccaagaccaccatgcggctcttctaccccgagtcagcccattttgacccccaggcagagaacaatccgaacacgctgctggtgctggtgcccttcaagcccgtggacttccagtggatggaggccatcctcagcgacaggaagagggttcgtaaagggttttggaagcagcccccactgatctgggatgccaacccggagcaagtgcgcatcctcaacccgtactacatggaagtaactgctgctaaactgctcagcctccccatgaagcagccaaggaaggtcaaacagaaaccaaccacggggctgttggccatcaccttggctctccacttctgcgacctggtgcacattgcaggctttgggtaccctgactcggccaacaagaagcaaaccatccactactatgagcagatcacactcaagtccatggctgcctcggagcacaacatctcacatgaggcggtggccatcaagcgcatgctggagctgggcctggtcaagaacctcacctacttctgagggcaatggggctgcgcagggacacgtccccaccttggagcccgcagtggtcctggaggagccggtgtcactgagctccccaccatgctggtggcagtatgggggccatgccagcttgctgaccccgggggtgcagggagccccttaatggggacttttcatatggaaacactgaaactagtgaggacctgggagggattctggggaggatggaggggaccttcccccagcaggtctggggggccacggcagggtgcactggagcccctctcttcccttggatactcttg

SEQ ID NO: 26 - protein, 335 aa, Gallus gallus
MPALLIKMINKSRGKILGVLALFLVMVWYSIYREDSFYFPVQENKTVCPIGEVERKAAQLIGNYTRDRPLFLQLKDYFWVRTPSLYELPYGIKGSEDVLLRLLSVTSYSLPESIQSLKCRRCVVVGNGHRLRNSSMGDTINTYDVVIRLNNAPVHGYEQDVGSKTTMRLFYPESAHFDPQAENNPNTLLVLVPFKPVDFQWMEAILSDRKRVRKGFWKQPPLIWDANPEQVRILNPYYMEVTAAKLLSLPMKQPRKVKQKPTTGLLAITLALHFCDLVHIAGFGYPDSANKKQTIHYYEQITLKSMAASEHNISHEAVAIKRMLELGLVKNLTYF

SEQ ID NO: 27 - DNA, 1296 bp, Gallus gallus
gccccgatgcggaggcgaagtgcccgggccgagcccagcgcagcaatgctgagtgacgataactctgtaaagctgaaaagtgattgtttgcctcctgtgcaatggtgtaaggtagctgcacatgaagatgagaagaccaatctggtttttaaaaggtactcgcaaatttcttgcactgtttgtgattggagggtgcttcctttatatcctcaaattacatttttaccctgaagaatgtgacagaacaaaaacaccgtatgtggactttgatcgcgtaaagagagcacaacaatatgccagtgctgtgttgcaggagcagtgccgaccttcgtatgtgaaaaaagcaatgggaaagttatttgcagagaaatacagcatggacatacctccctttgtaggaaaaaatatagatgatgatgaagctttatttaaatatggacctccgtttggattccacaggttctttgataagcttaaaaagcttctcgaactcttaccagagcacgatttgccagaggatttgaagtcaaaacactgtaagcgttgtgttgttattggcagtggtggaattctgtatggatcagagctaggccacttactgaatcagtatgatattgttataaggttaaatgatgcaccagttcaaggatacacggatcacgttggtaacaaaactactataaggatgacttacccagaaggagctccactttctgaacacgagtatccccctgctagtttatttgtggctgtcctctttaaaagtgttgatttcaattggcttcaagcaatggtaaaaaatgaaacactgtctctgtggatacgacttttcttttggaaggaagttgccaagaaaattccttttacatcaaaacaatttcggattctcaatccagtcatcgttaaagagacagccttggacatcctagagttccccaaacctcgatcaatattctggggttgggataagaacgtacccacaattggggtcatggcagtcgttctggccacacatctatgtgatgaagtaagcatagcaggatttggatacgacctcaaccagcccagcacacctttgcactattacaacaacctctgcatggctgccatgaacagacaaacgatgcacaatgtgacaggtgaaacaaaattactgcaaaaactggtcaaagaaaaagttgtgaaagacctcactggtggaatccattgtgaattctgcaacaaagacagctagtaattgaagtgcagcacggcctggatttgttaaattcccagaagttttgttggaacaacctt

SEQ ID NO: 28 - protein, 368 aa, Gallus gallus
MRRPIWFLKGTRKFLALFVIGGCFLYILKLHFYPEECDRTKTPYVDFDRVKRAQQYASAVLQEQCRPSYVKKAMGKLFAEKYSMDIPPFVGKNIDDDEALFKYGPPFGFHRFFDKLKKLLELLPEHDLPEDLKSKHCKRCVVIGSGGILYGSELGHLLNQYDIVIRLNDAPVQGYTDHVGNKTTIRMTYPEGAPLSEHEYPPASLFVAVLFKSVDFNWLQAMVKNETLSLWIRLFFWKEVAKKIPFTSKQFRILNPVIVKETALDILEFPKPRSIFWGWDKNVPTIGVMAVVLATHLCDEVSIAGFGYDLNQPSTPLHYYNNLCMAAMNRQTMHNVTGETKLLQKLVKEKVVKDLTGGIHCEFCNKDS

SEQ ID NO: 29 - DNA, 2352 bp, Gallus gallus
tttttgctttcaaacaaggcgatttttgttttgtttctgtcctttgccgccggcagagccatcgagttgtgtgtcgatgcgagcgttggggcgagcagagactttccaggaacccccggctgtctccgggccggtaacgccgggcgccacgggtgcagccctgtgccgtggcggctttctaggaccggttcctcccccgatgccccgtgggctttcgcgtgggtggcgatcgtcggaggcagcggcacggagagggagacccccgtgcccctcagcttgcctggcttttcgggtttttttgcttctcgagagggcgccccgcctcccctcgctgcccgggagggggtcagggctttccgcgggcgggcgagcagtcgctgtaaccgcagccgctgtgggaggacgcgcgggggatggagcggcgcggtgccttccctcacacgaggctctcctcccctggaaggcttcgatataagctgaggaggagcttcggcagacgcgagtcaaacgccacctccatcctaacgcgcagcatgcttccttccacgttttcttttgcagatggggttcagctgaacgaagccccgctcttcttcctcatcgttaccgtaagcgtgcggaagtcgggctggagctgcttgcggacggtacggcccctctaagaagctgcacgttaccttctctctgcggttcgatctgatttgaagtcccttccctgttgacaactggtctaccagtcatgaaacgaattcttctgtttttcatcctggctgctgctgttatgtacggtatattgcatggaaatctgtggagaaataacttctactggattagcttttatggacagacttcttctgtgaggggtccttcttccagtgaggctagtggagttacccagctgccacctacggctgtggagagaaggaacgccctaaacacttgcactctgaaaccagcatttgaatctttactggatgttgagaaaatatacccgttcctgtgtgccagtgattttatcagagtggcagagtaccatggaagtgataagttcgagctaccttatggaataaagagagcagaacaattttttcgttcagccctttcaaaactgcaaaattgtggactgtccaacaaagacgacagtgttgcctgccgacggtgtgttgtggtcggtaatggaggagtacttcgaaataagacgttaggagggaaaattgactcctatgatgtgataataagaatgaataatggccctgttatagggtacgaagaggatgttgggagaaggacgactttccgcctttcctacccagaatccatcttctcagatccaatccactacgaccctaacactactgttgttattatcgtcttcaaaccacgtgacttaaagtggctttgggagattttaggtggtcagaaaataagtgctaaaggcttttggaagaaaccagctctaaacatgatatataaatctaatcaaatcaggattcttgatcccagcatcaccagaaaaacagcttatgattggcttcatttcccaacaagatttcccaaaaaagagaaacccaagcatccaacaacggggctaattgccattacactagcatttcacatttgtcatgaagttcacctggcgggcttcaaatatgacttcactgacaggaacagttctttgcactactatggcaacgaaacaatgtctcagatgatgcagaatgaataccatgacatcagtgctgagcagaaattcttgaagaagcttatagacaagaactttgtggtcaacttgacgtgaaagctggatggaaaatctgaagaacagtcactactttcaagatttcagaatctttttattttttgtatgacatttttattttttaagtgcaacgtaactgtttactgttgaaaaacagcaaggaaacctcataaggcagaagcttcttctaagccagaggataatggactattctaagcagagctaactgaatttctgtgaagctatttaatgggaaaaacacaaaactttcagctgaaagtatcagggatgtataaaaatgtgattcacattacttatttatcagcaagaacttttttccaaacgattacttctctggaataccttcttttctaacgtcctccaaaaggattacactgttaagggactgagacaactatttaagtagtggatggatgtcaatgcttgaatctttcttgtaatacaaaagtggctttaaaagcaatgccttgaagtcattactatgcgtttgggagaaggggaaacatgcggaacatacagactttagtctgctctcctcctgagcagaggactcatccatccatcttctgtga

SEQ ID NO: 30 - protein, 352 aa, Gallus gallus
MKRILLFFILAAAVMYGILHGNLWRNNFYWISFYGQTSSVRGPSSSEASGVTQLPPTAVERRNALNTCTLKPAFESLLDVEKIYPFLCASDFIRVAEYHGSDKFELPYGIKRAEQFFRSALSKLQNCGLSNKDDSVACRRCVVVGNGGVLRNKTLGGKIDSYDVIIRMNNGPVIGYEEDVGRRTTFRLSYPESIFSDPIHYDPNTTVVIIVFKPRDLKWLWEILGGQKISAKGFWKKPALNMIYKSNQIRILDPSITRKTAYDWLHFPTRFPKKEKPKHPTTGLIAITLAFHICHEVHLAGFKYDFTDRNSSLHYYGNETMSQMMQNEYHDISAEQKFLKKLIDKNFVVNLT

SEQ ID NO: 31 - DNA, 2192 bp, Gallus gallus
gagaagtgccgcgctcggtggccagcaagctcgattagaagtgagatgagggcacgaagagctgcttgcatggcacagatgtggcagtggctcatctggatgtcacctggccacccccttgtgactggaagagtttgacaggagctgctgaggacagcgaaggatcctggagatgaaggtgtttcttgaactcttcagcgcctcacgcggctgttgtgtttggggatatcctgccttaaatcctggacaaaaatctgccaacagccaaaaatgacctgagtgggagagccactgctttgtccaacaggcccagcttcctgccccgtgctcactgtcccgtttggatgcgacgtacactatggttcacatcaatgtgctgaaaaaattcatgtgtgttcttgtggtgatactgatagctctgacggtttgcctgtggaaagagacaagaggaagctactatgttcctttgaagaacgatggcacacaggttcacagggctctggacaaatggaacctacttaaatcgcagggcctcctccatgaagctgctggtgaaatgggtcagatgcctaaagcattgcccaacaaccaaaacaaggtaaaaggcatcacctctggagcagtagagaagtccaggaaagcagcagagcacgtgaaggtatgggataaggacagctcgtccagaaacctcatacccaggctgcagaaggtcaggaagaactacctgtccatgaacaagtacaatgtgacttacaatgggaagatgaatgctgctaaactcagcccagagcagctgctgtgccggctgcgggacagggtgaacgtgaccatgatacggggatcggacggtccatttaattcctcagaatggcagcactacctgccagacaaaagcctcaatgagacggtgggccgcctgggtcgctgtgctgttgtgtcctcagcaggctctctgaaatcatctcacttgggaccagagatagatagccatgatgctgtcttgcggtttaatggggctcctgtcaaaggatttcaagaagatgtggggcaaaaaacgacgattcgtcttgtcaactcccagctggtcactgttgaggagcagcagttcctgaaagatgcgctgtataacactggaatcttgattgtctgggatccagcaccgtatcatgcagaaatccatgagtggtaccgaaaaccagactacaagttttttgaagcctacaagtcgtatcgtattagacatccggagcagcccttttatatcctgaacccaaagatgcagtggcaactctgggatattctgcaggagaattccctggagcatattcagcctaacccaccgtcttcaggaatgctgggcattgtgatcatgatgacgctctgtgatgaggtggatgtgtatgaatttctcccttctaagcggcagacggacatttgccactactaccagaagtttcacgaccatgcctgcaccatgggagcttaccaccctctcctgtttgagaagaacttggtgaagcatttaaaccaaggcaccgatgaggacatctatactcacgggaaggttactttgcctggcttccgaaatgtacattgctagcgaatcggtttttattgcatttagacttttgccttgactgttgaagcactttgaaaatcagggttgtcatatttgttaggtctaagcactggtgtgttttgacggctcttccgcgttgcaggactctgaggcaaagctcctccgaactggaaggcagtgcaatacgttggggtacatccacgcagcgtctgcgtgagcccatgcaataaatgtcctcctggtggggctgggctcgtgctgctggtggatatctgtcttcagctcaactgatgatgccgtgtgggaatcgcgggagatgctttctaagcagagactgcacaagtccgaatcgagtgctgataaagttcagcttgctccaaaggggttctgacagccgaaagcctgatctgctgagaacaagaagcattcttgaacacttgttcttatcgtccctggagctctttggagcagtgattgaaggaatcactgttaaagctgggtgctgataagctcatctcctagtccaaaaatatttgatcttaccattcagataagcaaggcagcttcctttcaaaaatataatgctttttaaattcaaaaaaaaaaaaaaaaa

SEQ ID NO: 32 - protein, 413 aa, Gallus gallus
MVHINVLKKFMCVLVVILIALTVCLWKETRGSYYVPLKNDGTQVHRALDKWNLLKSQGLLHEAAGEMGQMPKALPNNQNKVKGITSGAVEKSRKAAEHVKVWDKDSSSRNLIPRLQKVRKNYLSMNKYNVTYNGKMNAAKLSPEQLLCRLRDRVNVTMIRGSDGPFNSSEWQHYLPDKSLNETVGRLGRCAVVSSAGSLKSSHLGPEIDSHDAVLRFNGAPVKGFQEDVGQKTTIRLVNSQLVTVEEQQFLKDALYNTGILIVWDPAPYHAEIHEWYRKPDYKFFEAYKSYRIRHPEQPFYILNPKMQWQLWDILQENSLEHIQPNPPSSGMLGIVIMMTLCDEVDVYEFLPSKRQTDICHYYQKFHDHACTMGAYHPLLFEKNLVKHLNQGTDEDIYTHGKVTLPGFRNVHC

SEQ ID NO: 33 - DNA, 2274 bp, Gallus gallus
atgaaacctaacttgaagcaatggaaacaactcatgctgtttggaatctttgcatggggtctgctttttctagtgatcttcatctatttcacagacagcaacagtgctgagccagttcccagttccttttcttacattgaaacgaagaggctcctgcccctgcagggcaagcagagagtcatcatgggagccatacacgatccgtcattctctgaagccattgatgggaatgaggtactccttaatgaagatcttttagatacatttaaatcagagactggaagtattaagaaatggactgatttggaagatgcctttagaagcgaagatgagttttttccatcccagataggacgaaagtcaaaaagtgctttctaccaagtgaacgatgattatttattcgctgctggtcagcctatgtcacacaacagcttccaagagatagcaaaattcatctcagccgatgaggataatccaaaagaaagtattttacagaacaactggagccgtcagaggagaatgaggagaaggagcacaaagcacagaagaagccagatgcttgatgaatctgatgactgggatgggctgtattctacaatgtcgaaatcctttctatacaagctctggaaaggagatgtctcttccaagatgctgaaccctcgactgcagaaggcgatgaaagattatttgagcaccaataagcatggggtgcggttcaaagggaaacgaaactcgaagctgacaggcgaccagcttttctgcgagctgaaagaaagggtggatgtgaaaacaatagatggcaaggaagctcccttctccactcttggatgggagaagcacgttcctcaaattccactgggcaaattgtatacacatggctttgggagctgtgccgtagttatgtctgctggtgcgatactgaactcctctctaggggatgaaatagattctcatgatgctgttctaagatttaattctgctccaacacgcggctatgaaaaagatgttggaaataaaacaacgatgcgaatcattaactctcagattctcaccaacccaaaccatcactttgttgacagctccttgtacaaagatgttatcttagtagcctgggatcctgctccctactctgcaaatctgaatgtgtggtataagaagccggactacaatctgttcactccctatgtacagcatcgcaggaagaatccaaaccagcctttttacattctccatccaaagtttatatggcagctctgggacatcattcaggagaacacgaaagagaagatacagcccaaccctccttcttcaggttttattggtatcctcatcatgatgtccatgtgtaacgaagtgcacgtgtacgaatacatcccttcagtccgacagaccgacctatgtcactatcatgaactctactacgatgcagcttgtaccttaggggcctatcacccgctgctctatgagaagctgttggtgcagaggatgaacaaaggtttgcaggatgatctgtatcggaagggaaaggtcattttgccagggttcaagtctgtcaaatgcccggaacgaaataattttccacccttgtagaagagagtctttcacaaacaatgtgcaataaggtactactgtcgtactataaacaaggagagaatacttgaaaaatgtattagaccaacccagtcttgagtctataaattgtaattaagtagcaggcatgagaaatacttctttcctgagcctgagtatttattactgctttgcaaatagttaaagaaaacaaaaagcttagcttacaaaaggtgcagaggacatacttaggccgaaatataatgtattgttgtgggtgtgaccgtcagaatttgtcagtggtctcttgtgccacttatgctagatggtaacttttttttttttttaaaggaatttatttaagtgttaaatccagcattgtgaggcagcctgtatcgctcatgtacagagctgccagttgaacaatgcagcgtttctcatggctccatgggattttcacactctccaggaatgaagtaattgctactctgagctgaatattcattaattagaggagtctttcagttcctgttcatacactggttcacttgcaggcttctaactgtacaggaaaccttatggtggctatgaagtcagtgcagatgtaggaagcagaacacgcagctaaaccaattaaaccactggatgtacccttggtgtcacatcccattgctcacactgagcagggcagagggcaaagagaaa

SEQ ID NO: 34 - protein, 529 aa, Gallus gallus
MKPNLKQWKQLMLFGIFAWGLLFLVIFIYFTDSNSAEPVPSSFSYIETKRLLPLQGKQRVIMGAIHDPSFSEAIDGNEVLLNEDLLDTFKSETGSIKKWTDLEDAFRSEDEFFPSQIGRKSKSAFYQVNDDYLFAAGQPMSHNSFQEIAKFISADEDNPKESILQNNWSRQRRMRRRSTKHRRSQMLDESDDWDGLYSTMSKSFLYKLWKGDVSSKMLNPRLQKAMKDYLSTNKHGVRFKGKRNSKLTGDQLFCELKERVDVKTIDGKEAPFSTLGWEKHVPQIPLGKLYTHGFGSCAVVMSAGAILNSSLGDEIDSHDAVLRFNSAPTRGYEKDVGNKTTMRIINSQILTNPNHHFVDSSLYKDVILVAWDPAPYSANLNVWYKKPDYNLFTPYVQHRRKNPNQPFYILHPKFIWQLWDIIQENTKEKIQPNPPSSGFIGILIMMSMCNEVHVYEYIPSVRQTDLCHYHELYYDAACTLGAYHPLLYEKLLVQRMNKGLQDDLYRKGKVILPGFKSVKCPERNNFPPL

SEQ ID NO: 35 - DNA, 1292 bp, Gallus gallus
cgcgccccacgcctcctgtgaccctcgtgccccacggccgccccagctccgcgggataaagatgctggtccgcgtcttcgtcgtcctgctgtgcgcggccgcgctctccgtgctctacgtgctgctgtgccgcgaggccgccgggcagagggacggctccgcgtacaccgcgcccgcggcgctcagcttgcagggctacagccgcgtccccgacgggaagccgctgcgcagagctccgtgccgccgctgcgccgtggtctccagctcggggcagatgctgggatcgcacctgggccgggagatcgacgggcaggagtgcgtgctgcgcatgaaccacgcccccaccgccggcttcgaggaggacgtgggcacgcggagcaccgtccgcgtcgtgtcgcacaccagcgtcccgctgctgctcaggaaccagccctacttcttccagcagtcccgggacaccatctacgtcatttggggtcccagcaggaagatgagccgcgagaagggcggcccgacgcaccgagcgctgctcagggtgctggagatgtacccccgcctgcagctctacacgctgaccgaggagaagatggcgtattgcgacgacgtcttccagaacgagacaggcaagaacaggctgaaatccggctccttcctgagcacggggtggttcaccatgatcctggccatggagctgtgcgagcacatctgcgtcttcggcatggtcagcgacagctactgcagggagaagaaccactcgagcgtgccttaccactacttcgagaaggggcggctggatgagtgcaggatgtacctggtgcacgagagggccccccgcgccgggcaccgcttcatcaccgaaaaagccatcttctcccgctgggccaagaggaaggacatcatcttcagccacccgtcgtgggcaggggggtaggagaggcggcagtgcggttggaagcccctcgatatgcccggtattgggggtctgcagccactgggggggcagcagtggagaccccgttgcgttggagcgcataggacaggactggatccaccgagcccccccagctgcaggccccgagctggcttggacccgtgcagtgtggatgtttaatgtgggattcatcccgggacggaccccacgtatatggggcacgtggagcggggccgggaccccccgtcccacagacccccgtgtgccccctgcccgcagccctgagctgccttgccacaagtgcccttggattcagaccaaagccgacttgcccattaaaagcatttgtaagcccgaaaaa

SEQ ID NO: 36 - protein, 289 aa, Gallus gallus
MLVRVFVVLLCAAALSVLYVLLCREAAGQRDGSAYTAPAALSLQGYSRVPDGKPLRRAPCRRCAVVSSSGQMLGSHLGREIDGQECVLRMNHAPTAGFEEDVGTRSTVRVVSHTSVPLLLRNQPYFFQQSRDTIYVIWGPSRKMSREKGGPTHRALLRVLEMYPRLQLYTLTEEKMAYCDDVFQNETGKNRLKSGSFLSTGWFTMILAMELCEHICVFGMVSDSYCREKNHSSVPYHYFEKGRLDECRMYLVHERAPRAGHRFITEKAIFSRWAKRKDIIFSHPSWAGG

SEQ ID NO: 37 - DNA, 1525 bp, Gallus gallus
cctgcacggcggcgcttccccggccgagccatggccgcggctcccccgccatgccgagcctagcggggagcgggcggagcggcgcccgggaggcgcacaaaatgaagaccctgatgcgccacgggctggccgtctgcttggcgctcaccaccatgtgcaccagcttgttgctcatgtacggcggcatcggaggcggcggcgggggccacccggagcctcggcggcggcagcagcagcagcagcaggtggcggcggtgcccagccgccctccgggacgcggccagcaccgcccagcgctccccgtcggggccggactcctggagggctacatcagcgtcctggagcacaagcctttaaaaatgcactgcaagagctgtgcattggtaaccagttctggacaccttctgggaagtaaacaaggtgacagaatcgacgagacggagtgcgtaatacgaatgaatgatgcacctactcgaggttatggacaggatgttgggaacaaaacaagccttcgagtcattgcacactccagcattcagaggattttgcgaaatcgcaatgaactcttaaatatgagccacggtgctgtgttcatcttctggggtcctagcagctacatgaggagagatggtaaaggcttggtgtacaacaacctgcagctgatgaatcagatactgcctcaattaaaagcatacatgatttctcgccacaagatgcttcaatttgatgacctttttaaacgggaaactgggaaagacaggaagatatccaacacttggcttagcacgggctggttcacaatgactatcgccttagagctgtgtgacaggataaatgtttatggcatggtgccaccggatttctgcagggatcctaatcatctttcagtaccttatcattattatgaacctttgggacctgatgaatgcacaatgtacatttcacacgagcggggacgaaagggcagccatcatcggttcatcacagagaaacgagtgtttgagaactgggcgcggacattcaacattcacttcttccaaccagactggaaaccagaaccacttactgtaaatcaccccgagatgaaagcagcggtctgagggatgaatgcaaaagactgcaaccgcaatcaccgactgtatcagccatcagggggttggaccttctgggacagcaaggcaactgacagcaaaagggtaacgggatttgcagctgataactgcaacaagtcaggaagttccgatggaggggtatatagagagcactttctgttgaactgtgtgttaatccgctatatcgcctttctggccatctgacttcctgtacgtgtgtgtgatttgtgaaaagcaactcggtatcattacaggatgggtaattcattatggttttttaaagtacagcaccactgacttttcatagtgaaaactgatggtatttatttaatggaggtttttatgcaacctaggccagtatttttctaattcacagttctgtggtcgttgatctttcataatctttcaaatcc

SEQ ID NO: 38 - protein, 349 aa, Gallus gallus
MPSLAGSGRSGAREAHKMKTLMRHGLAVCLALTTMCTSLLLMYGGIGGGGGGHPEPRRRQQQQQQVAAVPSRPPGRGQHRPALPVGAGLLEGYISVLEHKPLKMHCKSCALVTSSGHLLGSKQGDRIDETECVIRMNDAPTRGYGQDVGNKTSLRVIAHSSIQRILRNRNELLNMSHGAVFIFWGPSSYMRRDGKGLVYNNLQLMNQILPQLKAYMISRHKMLQFDDLFKRETGKDRKISNTWLSTGWFTMTIALELCDRINVYGMVPPDFCRDPNHLSVPYHYYEPLGPDECTMYISHERGRKGSHHRFITEKRVFENWARTFNIHFFQPDWKPEPLTVNHPEMKAAV

SEQ ID NO: 39 - DNA, 30 bp, Artificial Sequence
  IRES insert
aattccccctctccctcccccccccctaac

SEQ ID NO: 40 - DNA, 1089 bp, Artificial  Sequence
  chicken beta 1,4- galactosyltransferase Type 1 codon optimized DNA sequence
atgaaagaacctgcacttcctggtacttcactgcaaagagcatgtagactgctggtagcattttgcgccctgcacctgagcgcaaccctgctctactacctggctggatccagcctgactccaccccgctctccagaacctccccctcggaggccgcctccagccaacctctccctgccaccctcccggcctcctcctccccctgcggctcgcccccgcccaggacctgtttctgcacaaccccggaacctgccagattctgcaccatctggactgtgccccgatccaagtccactgctcgttggtcctctgcgggtggagtttagtcagccagtgaacctggaggaagtggcttctaccaatccggaggtcagggaaggagggagattcgccccaaaggactgcaaagcgctccagaaggtggctattattatccccttcaggaacagagaggagcacctgaagtattggctgtactacatgcacccgattcttcagagacagcaattggactatggggtctatgtgattaatcaagacggcgatgaagaatttaacagagctaaactgcttaatgtcggtttcactgaggcactcaaggaatacgattatgattgctttgtgttttccgatgtggatctgattcctatggacgaccgtaacacatataagtgctatagtcaaccacgtcacctgagtgtgtcaatggacaagtttggctttaggctgccgtataaccagtatttcggaggagtttcagcattgagtaaagaacagtttacaaaaatcaacgggttcccaaataactactgggggtggggcggagaggacgacgacatctacaacagactggtttttaaggggatggggatttcccgcccggatgcagtaataggcaagtgtcgtatgatacgccatagcagggatagaaagaacgaacccaaccctgagcgctttgaccggattgcacatacaagagaaactatgtcatctgatggacttaactctctttcatatgaggtgctgagaacagatcggttccccctgtacactagaatcacagtagatatcggggcacctgggtcataa

SEQ ID NO: 41 - DNA, 1179 bp, Artificial Sequence
  chicken alpha-2,3-sialyltransferase Type 3 codon optimized DNA sequence
atgggtcttttggttttcatgagaaatctgctgctggctctgtgtctgttcctggtcctgggatttctgtactactctgcatggaagctccacctgctgcgctgggaggatagctctaaatatggacgcctgagccatagctcttttcctaagcaaagaccaagtgctgattctgtggtcttgtcatttgactctgttggacatactattggctctgaatatgacaaactgggttttctgcttaaccttgattctaaacttccccctgaattggcctcaaaatatgccaacttctctgagggagtgtgcaagcctggttatgcatctgccctgatgactgtgattttccctaaattctccaaacctgcccccatgttccttgatgactccttccggcgctgggcccgcattagagactttgtgcctccatttggcattaaagggcaggacaatctgataaaggcaatactgtctgctacaaaagattacagactcacaccagcactggacagcttgtcatgccgccgctgtatcattgttgggaatggtggtgttctggccaacaagagtttgggtcttaagattgatgactatgatgtggtcgttcgcctgaactctgcacctgtcaaaggctttgagaaagatgttggtggaaagacaacactgcggatcacttacccagagggggctattcagaagatggaacagtatgagaaagactccctgtttgtgctggcgggatttaaatggcaagactttaagtggctgaaatatattgtgtataaagaaaaggtctcagcttctgatggcttctggaaatcagtggctacccgggtgcctcgggagccacatgaaattcgcatactgaatccctatttcatccaagaagctgctttttcattcattggcctgccattcaataatggtctgatgggtcgggggaatatccccaccctgggttctgtggccatcacaatggctctgcataattgtgatgaggtggctgttgctggctttggatatgacatgagttcccctaatgctcccctgcattactatgagaacataaaaatgagtgccattaaggagtcatggactcataatatacaacgggagaaggaatttcttcgcaagctggttaaagccagagtgattacagatcttacatctgggatatga

(19) United States
(12) Patent Application Publication
(10) Pub. No.: US 2014/0298503 A1
(43) Pub. Date: Oct. 2, 2014

(54) Glycosylation in Avians

(71) Applicant: SYNAGEVA BIOPHARMA CORP., Lexington, MA (US)

(72) Inventor: Alex J. Harvey, Athens, GA (US)

(73) Assignee: Synageva BioPharma Corp., Lexington, MA (US)

(21) Appl. No.: 13/854,471

(22) Filed: Apr. 1, 2013

(51) Int. Cl. A01K 67/027

(52) U.S. Cl. CPC A01K 67/0275
USPC 800/19

(57) ABSTRACT

Transgenic avians which produce proteins in their oviduct tissue having modified oligosaccharide structures and methods of making such avians are disclosed herein. The invention also includes the modified proteins produced in the transgenic birds.

### RELATED APPLICATIONS

This application is a divisional of U.S. application Ser. No. 12/319,396, filed Jan. 7, 2009, which claims the benefit of U.S. Provisional Application No. 61/010,207, filed Jan. 7, 2008. The entire teachings of the above applications are incorporated herein by reference.

### BACKGROUND

Certain proteins with potential commercial uses can require post-translational modifications that are efficiently produced by mammalian cells. However, mammalian cells, such as the industry standard Chinese Hamster Cells (CHO), can be difficult to grow under GMP conditions and require immense resources to propagate at the scale needed for commercial purposes. Animal based bioreactors systems are an attractive alternative to CHO and other mammalian cell based systems due to reasons which include low cost, low maintenance and ease of scalability. However, the post-translational modification of therapeutic proteins, in particular glycosylation, is executed differently in certain animals and plants as compared to mammalian cells such as CHO cells. Transgenic avians, in part because of their prolific egg laying and protein production abilities, have been successfully employed as therapeutic protein bioreactors. In some instances, sugar molecules (i.e., oligosaccharide or glycosylation structures) attached to proteins produced in the oviduct of avians such as chickens and deposited into eggs have been found to have basic structure similar to CHO and human proteins. However, there are some structural carbohydrate elements that are not present on certain proteins produced in the oviduct that can be important for bioactivity and bioavailability in human patients.

The egg white is formed around the yolk as it traverses the oviduct, the avian equivalent of the mammalian fallopian tube. The region of the oviduct in which egg white formation happens is called the magnum and is populated by cells called tubular gland cells (TGCs) which specialize in the synthesis and secretion of egg white proteins.

The two primary classes of glycosylation structures found on proteins, N- and O-linked oligosaccharides, are synthesized by different sets of enzymes. For O-linked oligosaccharides (also referred to as O-glycans) produced in the magnum of laying hens and deposited in the egg white, the enzymatic machinery for oligosaccharide production appears to be similar to that for human O-glycan production, since essentially the same sugars and linkages are present in oligosaccharide structures produced in both humans and in the avian oviduct.

Hen egg white N-linked oligosaccharides (also referred to as N-glycans) have a structure somewhat similar to those found in humans but are typically lacking the terminal galactose and sialic acid sugars. For certain therapeutic proteins, having the terminal galactose and sialic acid can be important for bioavailability and thus efficacy in patients.

Terminal sialic acid residues, which are rarely present or not present at all on N-glycan structures produced in the hen oviduct, shields the N-glycan from recognition by various lectins (receptors that recognize sugar molecules). Proteins with terminal Gal can be bound by lectins expressed in the liver and cleared from the blood circulation in patients (Ashwell and Morell. Adv Enzymol Relat Areas Mol Biol 41: 99-128, 1974). Proteins with the N-glycan having terminal GlcNac, as is typically the case in proteins produced in the hen oviduct, or mannose are bound by lectins expressed on macrophages, also leading to clearance (Schlesinger, et al. Biochem J 192: 597-606, 1980). These results can lead to proteins having a short half-life which often reduces efficacy.

Interestingly, N-glycans produced in other organs in the chicken such as those found in the blood are typically terminated with Gal and/or sialic acid (Ito, et al. Rapid Commun Mass Spectrom 20: 3557-65, 2006; Raju, et al. Glycobiology 10: 477-86, 2000). Thus it is apparent that the chicken genome contains genes that encode all of the enzymes needed to synthesize a fully sialylated N-glycan.

For chicken egg white derived N-glycans, a small percentage of the branches are occupied by Gal and a small percentage of those Gals are capped with sialic acid. For the egg white O-glycans, a high percentage of branches are capped by sialic acid. There is a substantial amount of galactose and sialic acid in egg white proteins, predominantly due to the abundance of O-glycan modified egg white proteins (Feeney, et al. J Biol Chem 235: 2633-7, 1960; Feeney, et al. J Biol Chem 235: 2307-11, 1960; Robinson and Monsey. Biochem J 147: 55-62, 1975). N- and O-glycan synthesis pathways share the same pools of Gal and sialic acid (Varki, et al., Essentials of Glycobiology. Plainview, N.Y., Cold Spring Harbor Laboratory Press, 1999). Thus the levels of Gal and sialic acid that are available for glycan synthesis in TGCs are high and should not be a limiting factor.

The structure of the egg white N-glycans in addition to what is known about the relevant enzymes in mammals gives clues as to the cellular mechanisms that give rise to the egg white N-glycan structures. In mammals, N-glycan synthesis begins in the endoplasmic reticulum with the synthesis of the dolichol oligosaccharide precursor which includes two GlcNac residues and a number of mannose and glucose residues. This complex is attached to the asparagine of the target protein. The precursor is trimmed back to 3 mannose and 2 GlcNac residues by various glycosidases (termed the core pentasaccharide). GlcNac, Gal and sialic acid residues are then sequentially added by glycosyltransferases. It is at this stage that the diversity of N-glycan structures becomes prominent possibly due to the intracellular levels of the various glycosyltransferases and competition between the glycosyltransferases for free acceptor sites on the growing N-glycan branches (Varki, et al., Essentials of Glycobiology. Plainview, N.Y., Cold Spring Harbor Laboratory Press, 1999).

Starting with GlcNac, there are at least six N-acetylglucosaminyltransferases (GnTs) responsible for the addition of GlcNac to the trimannosyl core of N-glycans. The high level of branching of egg white N-glycans indicates that all six GnTs may be expressed in oviduct cells of the hen to some extent.

The galactosyltransferases (e.g., β1,4 galactosyltransferases), referred to as GalTs herein, are a family of at least 7 members which have distinct as well as overlapping roles in the formation of N- and O-glycans. GalT1 (type 1) is thought to be primarily responsible for addition of Gal to the GlcNac residues of all linkages on the N-glycan (Lee, et al. J Biol Chem 276: 13924-34, 2001). The other members of the family, in particular types 2 and 3, are thought to be able to catalyze this transfer though their actual role in N-glycan synthesis appears to be minor. GalT1 is typically expressed in a ubiquitous manner in all cell types, though the levels can vary.

The sialyltransferase (SialT) family catalyzes addition of sialic acid to Gal or N-acetylgalactosamine (GalNac) (in the case of O-linked glycans) as well as other acceptors. With respect to N- and O-glycans, the sialic acid addition is produced by either an α2,3 or α2,6 linkage depending on the specific SialT involved. Human N-glycans can have either or both α2,3 and α2,6 linkages. CHO-produced N-glycans have only the α2,3 linkage, due to a lack of expression of the α2,6 SialTs (Lee, et al. J Biol Chem 264: 13848-55, 1989). Egg white N-glycans and O-glycans also appear to be linked only through the α2,3 linkage.

There are six members of the α2,3 SialT family. Types 1 and 2 may be involved in O-glycan synthesis as they use the Gal-GalNac chain as an acceptor. Types 3, 4 and 6 apparently can add sialic acid to chains ending in Gal-GlcNac and may be involved in N-glycan and O-glycan synthesis. Type 5 appears to not be involved in O-glycan or N-glycan synthesis but rather may be involved in the addition of sialic acid to ceramide-containing compounds (Harduin-Lepers, et al. Biochimie 83: 727-37, 2001). Very little has been known about the avian α2,3 SialT family other than the expression analysis of type 1 (SialT1) in chick embryos (Kurosawa, et al. Biochim Biophys Acta 1244: 216-22, 1995).

It is currently estimated that the level for Gal at the last (i.e., terminal) or penultimate (i.e., second to last) position in egg white glycans is less than about 10% and the level for terminal sialic acid is less than about 2%. What is needed are birds which produce glycosylated proteins in oviduct tissue, such as magnum tissue, where a greater quantity of galactose and/or sialic acid is added to the N-linked oligosaccharides.

### SUMMARY OF THE INVENTION

It has been discovered that the key enzyme involved in transfer of Gal to N-glycans is not expressed in TGCs. This is particularly significant since sialic acid is only attached to N-glycans through a Gal residue. It has also been discovered that the enzymes that transfer sialic acid to Gal on N-glycans are expressed but at levels that appear to preclude efficient sialylation. These discoveries in part have lead to the invention of transgenic birds that produce therapeutic proteins (e.g., human therapeutic proteins) having oligosaccharide structures (e.g., N-linked oligosaccharide structures) with a more complete complement of terminal sialic acid residues and Gal (e.g., penultimate Gal) residues. These birds are often referred to herein “transgene-augmented glycosylation” birds.

The invention includes transgenic avians (e.g., transgenic chickens) containing a transgene in their genome which contains a glycosyltransferase coding sequence which is expressed. The invention also includes methods of making the transgenic avians. The oviduct tissue, for example, magnum tissue (e.g., tubular gland cells) of the transgenic avian can produce protein (e.g., an exogenous protein, for example, a therapeutic protein) having an N-linked oligosaccharide with at least one saccharide that would not be present in the absence of the transgene. Also included in the invention are proteins having modified oligosaccharide patterns produced as disclosed herein.

In one embodiment, the glycosyltransferase is an N-acetylglucosaminyltransferase, for example, an N-acetylglucosaminyltransferase 3 and the sugar is N-acetylglucosamine.

In another embodiment, the glycosyltransferase is a galactosyltransferase (e.g., galactosyltransferase type 1) and the saccharide is galactose. In one embodiment, the exogenous protein (e.g., therapeutic protein) produced in the oviduct of transgene-augmented galactosyltransferase (e.g., galactosyltransferase type 1) birds can be used as a substrate for addition of sialic acid. For example, using well known in vitro methods, sialic acid is linked to Gal that has been added to the oligosaccharide structures by the recombinant or exogenous galactosyltransferase in the oviduct.

In another embodiment, the glycosyltransferase is a sialyltransferase (e.g., a sialyltransferase type 3) and the saccharide is sialic acid.

In one embodiment, cells of oviduct tissue of the transgenic avians of the invention secrete the protein in the presence of egg white.

In one embodiment, transgenes of the invention include at least one of an oviduct specific promoter and at least a portion of a retrovirus such as an LTR.

One aspect of the invention relates to isolating or purifying the protein having the altered oligosaccharide pattern.

In one particular embodiment, the invention is directed to methods of producing a protein in an avian wherein the protein is exogenous to the avian. The method can include producing a transgenic avian containing a transgene encoding a glycosyltransferase wherein oviduct tissue of the avian produces an exogenous protein encoded by a second transgene and having an N-linked oligosaccharide. The N-linked oligosaccharide will have at least one of a galactose and a sialic acid attached to it wherein the oligosaccharide would not have the galactose and/or sialic acid attached in the absence of the transgene encoding the glycosyltransferase.

The invention includes transgenic avians containing transgenes having coding sequences for enzyme(s) involved in the synthesis of oligosaccharide structures that are found to be present in relatively low quantities in the hen oviduct tissue such as the magnum (e.g., tubular gland cells). For example, the enzymes may be present in oviduct tissue in quantities less than that found in other tissue in the bird. For example, the enzymes may be present in oviduct tissue in an amount less than about 90% that found on average in other tissue in the avian such as liver and kidney tissue, or for example, the enzymes may be present in oviduct tissue in an amount less than about 80% that found on average in other tissue in the avian such as liver and kidney tissue, or for example, the enzymes may be present in oviduct tissue in an amount less than about 70% that found on average in other tissue in the avian such as liver and kidney tissue, or for example, the enzymes may be present in oviduct tissue in an amount less than about 60% that found on average in other tissue in the avian such as liver and kidney tissue, or for example, the enzymes may be present in oviduct tissue in an amount less than about 50% that found on average in other tissue in the avian such as liver and kidney tissue, or for example, the enzymes may be present in oviduct tissue in an amount less than about 30% that found on average in other tissue in the avian such as liver and kidney tissue, or for example, the enzymes may be present in oviduct tissue in an amount less than about 20% that found on average in other tissue in the avian such as liver and kidney tissue, or for example, the enzymes may be present in oviduct tissue in an amount less than about 10% that found on average in other tissue in the avian such as liver and kidney tissue.

The invention also includes vectors that contain transgenes of the invention. Vectors used in accordance with the invention are designed to integrate transgenes of the invention into the chicken genome and express enzyme(s) in the cells of the oviduct that make egg white proteins. Any useful vector may be employed to produce the avians of the invention such as the transgene-augmented glycosylation avians. Some useful vectors include viral vectors such as retroviral vectors and adenoviral vectors, plasmids and other nucleotide sequences that can become part of the avian genome (i.e., integrated into the genome).

Other useful vectors such as non-infective nucleic acid vectors are contemplated for use herein. For example, site directed DNA integration, integrase mediated integration and artificial chromosomes are also contemplated for use in accordance with the invention.

Examples of avian retroviruses which are contemplated for use in accordance with the invention include, without limitation, Avian Leukemia/Leukosis Viruses (ALV), for example, and without limitation, RAV-0, RAV-1, RAV-2; Avian Sarcoma Viruses (ASV); Avian Sarcoma/Acute Leukemia Viruses (ASLV) including, without limitation, Rous Sarcoma Virus (RSV); Fujinami Sarcoma Viruses (FSV); Avian Myeloblastosis Viruses (AMV); Avian Erythroblastosis Viruses (AEV); Avian Myelocytomatosis Viruses (MCV), for example, and without limitation, MC29; Reticuloendotheliosis Viruses (REV), for example, and without limitation, Spleen Necrosis Virus (SNV). The invention also contemplates the use of Murine Leukemia Viruses (MLV); Molony Murine Sarcoma Viruses (MMSV); Moloney Murine Leukemia Viruses (MMLV); and lentiviruses (e.g., human immunodeficiency virus (HIV), Equine Infectious Anemia Virus (EIAV), feline immunodeficiency virus (FIV), bovine immunodeficiency virus (BIV) and simian immunodeficiency virus (SIV), and replication deficient forms of these retroviruses. Typically, retroviral vectors used in accordance with the invention are replication-deficient.

Other methods may also be employed to produce transgene-augmented glycosylation avians where infective DNA is not required to produce germline transmission, such as those reported in de Lavoir et al, Jun. 8, 2006, Nature vol. 441, p 766-769, the disclosure of which is incorporated in its entirety herein by reference.

In one embodiment, the invention is directed to transgene-augmented glycosylation birds that contain a GalT1, GalT2, GalT3, GalT4, GalT5, GalT6 and/or GalT7 encoding transgene in their genome and produce recombinant proteins, such as therapeutic proteins, in the oviduct tissue, e.g., magnum tissue (for example, in tubular gland cells), which carry N-glycans that are completely or substantially occupied by galactose at the terminal positions. For example, the exogenous proteins (e.g., therapeutic proteins) produced in accordance with the invention can have an N-glycan structure that is about 30% occupied by galactose at the terminal positions, or for example, the exogenous proteins can have an N-glycan structure that is about 40% occupied by galactose at the terminal positions, or for example, the exogenous proteins can have an N-glycan structure that is about 50% occupied by galactose at the terminal positions, or for example, the exogenous proteins can have an N-glycan structure that is about 60% occupied by galactose at the terminal positions, or for example, the exogenous proteins can have an N-glycan structure that is about 70% occupied by galactose at the terminal positions, or for example, the exogenous proteins can have an N-glycan structure that is about 80% occupied by galactose at the terminal positions, or for example, the exogenous proteins can have an N-glycan structure that is about 90% occupied by galactose at the terminal positions, or for example, the exogenous proteins can have an N-glycan structure that is about 95% occupied by galactose at the terminal positions, or for example, the exogenous proteins can have an N-glycan structure that is 100% occupied by galactose at the terminal positions.

In one embodiment, the invention is directed to transgene-augmented glycosylation birds that contain one or more GalT1, GalT2, GalT3, GalT4, GalT5, GalT6 and GalT7 encoding transgene(s) in their genome and produce recombinant proteins, such as therapeutic proteins, in the oviduct tissue, e.g., magnum tissue (for example, in tubular gland cells), which carry N-glycans that are completely or substantially occupied by galactose at the penultimate positions. For example, the exogenous proteins (e.g., therapeutic proteins) produced in accordance with the invention can have N-glycan structures that are about 30% occupied by galactose at the penultimate positions, or for example, the exogenous proteins can have N-glycan structures that are about 40% occupied by galactose at the penultimate positions, or for example, the exogenous proteins can have N-glycan structures that are about 50% occupied by galactose at the penultimate positions, or for example, the exogenous proteins can have N-glycan structures that are about 60% occupied by galactose at the penultimate positions, or for example, the exogenous proteins can have N-glycan structures that are about 70% occupied by galactose at the penultimate positions, or for example, the exogenous proteins can have N-glycan structures that are about 80% occupied by galactose at the penultimate positions, or for example, the exogenous proteins can have N-glycan structures that are about 90% occupied by galactose at the penultimate positions, or for example, the exogenous proteins can have N-glycan structures that are about 95% occupied by galactose at the penultimate positions, or for example, the exogenous proteins can have N-glycan structures that are 100% occupied by galactose at the penultimate positions.

In one embodiment, the invention is directed to transgene-augmented glycosylation birds that contain one or more GalT1, GalT2, GalT3, GalT4, GalT5, GalT6 and GalT7 encoding transgene(s) in their genome and produce recombinant proteins, such as therapeutic proteins, in the oviduct tissue, e.g., magnum tissue (for example, in tubular gland cells), which carry N-glycans that are completely or substantially occupied by sialic acid at the terminal positions. For example, the exogenous proteins can have an N-glycan structure that is about 30% occupied by sialic acid at the terminal positions, or for example, the exogenous proteins can have an N-glycan structure that is about 40% occupied by sialic acid at the terminal positions, or for example, the exogenous proteins can have an N-glycan structure that is about 50% occupied by sialic acid at the terminal positions, or for example, the exogenous proteins can have an N-glycan structure that is about 60% occupied by sialic acid at the terminal positions, or for example, the exogenous proteins can have an N-glycan structure that is about 70% occupied by sialic acid at the terminal positions, or for example, the exogenous proteins can have an N-glycan structure that is about 80% occupied by sialic acid at the terminal positions, or for example, the exogenous proteins can have an N-glycan structure that is about 90% occupied by sialic acid at the terminal positions, or for example, the exogenous proteins can have an N-glycan structure that is about 95% occupied by sialic acid at the terminal positions, or for example, the exogenous proteins can have an N-glycan structure that is about 100% occupied by sialic acid at the terminal positions.

In one embodiment, the invention is directed to transgene-augmented glycosylation birds that contain one or GalT1, GalT2, GalT3, GalT4, GalT5, GalT6 and GalT7 encoding transgene(s) in their genome and produce recombinant proteins, such as therapeutic proteins, in the oviduct tissue, e.g., magnum tissue (for example, in tubular gland cells) where one out of three GlcNac residues of the oligosaccharide has a galactose residue attached, or for example, where two out of three GlcNac residues of the oligosaccharide has a galactose residue attached, or for example, where three out of three GlcNac residues of the oligosaccharide has a galactose residue attached, or for example, where one out of four GlcNac residues of the oligosaccharide has a galactose residue attached, or for example, where two out of four GlcNac residues of the oligosaccharide has a galactose residue attached, or for example, where three out of four GlcNac residues of the oligosaccharide has a galactose residue attached, or for example, where four out of four GlcNac residues of the oligosaccharide has a galactose residue attached, or for example, where one out of five GlcNac residues of the oligosaccharide has a galactose residue attached, or for example, where two out of five GlcNac residues of the oligosaccharide has a galactose residue attached, or for example, where three out of five GlcNac residues of the oligosaccharide has a galactose residue attached, or for example, where four out of five GlcNac residues of the oligosaccharide has a galactose residue attached, or for example, where five out of five GlcNac residues of the oligosaccharide has a galactose residue attached.

In one embodiment, the invention is directed to transgene-augmented glycosylation birds that contain one or more SialT1, SialT2, SialT3, SialT4, SialT5 and SialT6 encoding transgene(s) in their genome and produce recombinant proteins, such as therapeutic proteins, in the oviduct tissue, e.g., magnum tissue (for example, in tubular gland cells) where one out of one galactose residues of the oligosaccharide has a terminal sialic acid residue attached, or for example, where one out of two galactose residues of the oligosaccharide has a terminal sialic acid residue attached, or for example, where two out of two galactose residues of the oligosaccharide has a terminal sialic acid residue attached, or for example, where one out of three galactose residues of the oligosaccharide has a terminal sialic acid residue attached, or for example, where two out of three galactose residues of the oligosaccharide has a terminal sialic acid residue attached, or for example, where three out of three galactose residues of the oligosaccharide has a terminal sialic acid residue attached, or for example, where one out of four galactose residues of the oligosaccharide has a terminal sialic acid residue attached, or for example, where two out of four galactose residues of the oligosaccharide has a terminal sialic acid residue attached, or for example, where three out of four galactose residues of the oligosaccharide has a terminal sialic acid residue attached, or for example, where four out of four galactose residues of the oligosaccharide has a terminal sialic acid residue attached, or for example, where one out of five galactose residues of the oligosaccharide has a terminal sialic acid residue attached, or for example, where two out of five galactose residues of the oligosaccharide has a terminal sialic acid residue attached, or for example, where three out of five galactose residues of the oligosaccharide has a terminal sialic acid residue attached, or for example, where four out of five galactose residues of the oligosaccharide has a terminal sialic acid residue attached, or for example, where five out of five galactose residues of the oligosaccharide has a terminal sialic acid residue attached, or for example, where one out of six galactose residues of the oligosaccharide has a terminal sialic acid residue attached, or for example, where two out of six galactose residues of the oligosaccharide has a terminal sialic acid residue attached, or for example, where three out of six galactose residues of the oligosaccharide has a terminal sialic acid residue attached, or for example, where four out of six galactose residues of the oligosaccharide has a terminal sialic acid residue attached, or for example, where five out of six galactose residues of the oligosaccharide has a terminal sialic acid residue attached, or for example, where six out of six galactose residues of the oligosaccharide has a terminal sialic acid residue attached.

In one embodiment, the invention provides for transgenic hens containing one or more SialT1, SialT2, SialT3, SialT4, SialT5 and SialT6 encoding transgene(s) in their genome which produce N-glycans in their oviduct tissue such as magnum tissue (for example, in tubular gland cells) with an increased percentage of branches ending with sialic acid relative to a non-transgenic bird. For example, the exogenous proteins can have an N-glycan structure that is 30% occupied by sialic acid at the terminal positions, or for example, the exogenous proteins can have an N-glycan structure that is 40% occupied by sialic acid at the terminal positions, or for example, the exogenous proteins can have an N-glycan structure that is 50% occupied by sialic acid at the terminal positions, or for example, the exogenous proteins can have an N-glycan structure that is 60% occupied by sialic acid at the terminal positions, or for example, the exogenous proteins can have an N-glycan structure that is 70% occupied by sialic acid at the terminal positions, or for example, the exogenous proteins can have an N-glycan structure that is 80% occupied by sialic acid at the terminal positions, or for example, the exogenous proteins can have an N-glycan structure that is 90% occupied by sialic acid at the terminal positions, or for example, the exogenous proteins can have an N-glycan structure that is 95% occupied by sialic acid at the terminal positions, or for example, the exogenous proteins can have an N-glycan structure that is 100% occupied by sialic acid at the terminal positions.

In one embodiment, the invention is directed to transgene-augmented glycosylation birds that contain one or more GalT1, GalT2, GalT3, GalT4, GalT5, GalT6, GalT7, SialT1, SialT2, SialT3, SialT4, SialT5 and SialT6 encoding transgenes, for example, GalT1 and SialT3 encoding transgene(s) in their genome and produce recombinant proteins, such as therapeutic proteins, in the oviduct tissue, e.g., magnum tissue (for example, in tubular gland cells) where one out of one galactose residues of the oligosaccharide has a terminal sialic acid residue attached, or for example, where one out of two galactose residues of the oligosaccharide has a terminal sialic acid residue attached, or for example, where two out of two galactose residues of the oligosaccharide has a terminal sialic acid residue attached, or for example, where one out of three galactose residues of the oligosaccharide has a terminal sialic acid residue attached, or for example, where two out of three galactose residues of the oligosaccharide has a terminal sialic acid residue attached, or for example, where three out of three galactose residues of the oligosaccharide has a terminal sialic acid residue attached, or for example, where one out of four galactose residues of the oligosaccharide has a terminal sialic acid residue attached, or for example, where two out of four galactose residues of the oligosaccharide has a terminal sialic acid residue attached, or for example, where three out of four galactose residues of the oligosaccharide has a terminal sialic acid residue attached, or for example, where four out of four galactose residues of the oligosaccharide has a terminal sialic acid residue attached, or for example, where one out of five galactose residues of the oligosaccharide has a terminal sialic acid residue attached, or for example, where two out of five galactose residues of the oligosaccharide has a terminal sialic acid residue attached, or for example, where three out of five galactose residues of the oligosaccharide has a terminal sialic acid residue attached, or for example, where four out of five galactose residues of the oligosaccharide has a terminal sialic acid residue attached, or for example, where five out of five galactose residues of the oligosaccharide has a terminal sialic acid residue attached, or for example, where one out of six galactose residues of the oligosaccharide has a terminal sialic acid residue attached, or for example, where two out of six galactose residues of the oligosaccharide has a terminal sialic acid residue attached, or for example, where three out of six galactose residues of the oligosaccharide has a terminal sialic acid residue attached, or for example, where four out of six galactose residues of the oligosaccharide has a terminal sialic acid residue attached, or for example, where five out of six galactose residues of the oligosaccharide has a terminal sialic acid residue attached, or for example, where six out of six galactose residues of the oligosaccharide has a terminal sialic acid residue attached.

In one embodiment, the invention provides for transgenic hens containing more than one of GalT1, GalT2, GalT3, GalT4, GalT5, GalT6, GalT7, SialT1, SialT2, SialT3, SialT4, SialT5 and SialT6 encoding transgenes, for example, GalT1 and SialT3 encoding transgenes, in their genome which produce exogenous protein in their oviduct tissue such as magnum tissue (for example, in tubular gland cells) with an increased percentage of branches ending with sialic acid relative to a non-transgenic bird. For example, the exogenous proteins can have an N-glycan structure that is 20% occupied by sialic acid at the terminal positions, or for example, the exogenous proteins can have an N-glycan structure that is 30% occupied by sialic acid at the terminal positions, or for example, the exogenous proteins can have an N-glycan structure that is 40% occupied by sialic acid at the terminal positions, or for example, the exogenous proteins can have an N-glycan structure that is 50% occupied by sialic acid at the terminal positions, or for example, the exogenous proteins can have an N-glycan structure that is 60% occupied by sialic acid at the terminal positions, or for example, the exogenous proteins can have an N-glycan structure that is 70% occupied by sialic acid at the terminal positions, or for example, the exogenous proteins can have an N-glycan structure that is 80% occupied by sialic acid at the terminal positions, or for example, the exogenous proteins can have an N-glycan structure that is 90% occupied by sialic acid at the terminal positions, or for example, the exogenous proteins can have an N-glycan structure that is 95% occupied by sialic acid at the terminal positions, or for example, the exogenous proteins can have an N-glycan structure that is 100% occupied by sialic acid at the terminal positions.

In one embodiment, proteins of the invention have oligosaccharides with between 1 and 5 sialic acids (e.g., between 1 and 4 sialic acids).

In one embodiment, proteins of the invention have oligosaccharides with between 1 and 5 galactoses (e.g., between 1 and 4 galactoses).

To produce transgene-augmented glycosylation birds that produce exogenous proteins the transgene-augmented glycosylation birds can be crossed with existing birds which are transgenic for therapeutic protein production in the oviduct where the efficacy of the therapeutic protein can be enhanced by having galactose and/or sialic acid at the oligosaccharide added to the therapeutic proteins. The transgene-augmented glycosylation birds can also be used to produce egg or embryo donors. Transgenes encoding proteins such as therapeutic proteins can be introduced into the embryos, for example, by methods known in the art, to produce lines of avians which will manufacture the transgene encoded proteins in the oviduct where the glycosylated transgene encoded proteins can have additional sugars such as galactose and sialic acid to the their oligosaccharide structures. In another embodiment, existing birds which are transgenic for therapeutic protein production are used to produce egg or embryo donors and a vector(s) encoding a glycosyltransferase(s) (e.g., GalT, SialT) transgene(s) is introduced into the donor egg or embryo.

In one embodiment, the invention is directed to producing hens such as chicken hens to make oligosaccharide structures in egg white-derived therapeutic proteins that more closely resemble the oligosaccharide structures naturally present on mammalian proteins, in particular human proteins.

First generation transgenic birds produced in accordance with the invention typically are referred to as the G0 generation and are usually hemizygous for each inserted transgene. The G0 generation may be bred to non-transgenic birds to give rise to fully transgenic G1 offspring which are also hemizygous for the transgene. The G1 hemizygous offspring may be bred to non-transgenic birds giving rise to G2 hemizygous offspring or may be bred together to give rise to G2 offspring homozygous for the transgene. Descendents of G0 birds which are hemizygous or homozygous for the transgene can be bred to descendents of G0 birds which are hemizygous or homozygous for another transgene to produce offspring hemizygous for both transgenes. The double hemizygous birds can be interbred to produce birds homozygous for one or both transgenes. These are merely examples of certain useful breeding schemes. The present invention contemplates the employment of any useful breeding scheme such as those known to individuals of ordinary skill in the art.

Any combination of features described herein is included within the scope of the present invention provided that the features included in any such combination are not mutually inconsistent. Such combinations will be apparent based on this specification and on the knowledge of one of ordinary skill in the art.

### BRIEF DESCRIPTION OF THE FIGURES

FIGS. 1 A and B show exemplary structures of an N-glycan which is attached to egg white proteins as occur naturally (1A) and in accordance with the invention (1B), Gal is occasionally found to occupy a terminal GlcNac residue and appears to be β1,4-linked as shown in FIG. 1A. On naturally occurring egg white proteins, Gal has not been detected as being present on a bisecting GlcNac. In addition, it appears that when a Gal is present on an N-linked oligosaccharide it will sometimes, though rarely be sialated. With the addition of Gal to terminal GlcNac residues in accordance with the invention, more of the terminal GlcNac residues are sialated than would otherwise be sialated in the absence of the transgene-augmented glycosylation. FIG. 1B shows an exemplary N-linked oligosaccharide structure present on a protein produced in the avian oviduct in accordance with the invention. In this exemplary diagrammatic and non-limiting structure all of the terminal GlcNac residues (except for the bisecting GlcNac) have an attached Gal which is sialated.

FIG. 2 shows an expression analysis of chicken galactosyltransferases. mRNA was isolated from cultured fibroblasts (F), magnum (M), liver (L) and kidney (K) tissues of a laying hen and analyzed by the Northern blot method. Blots were probed with sequences complementary to the chicken β1,4 galactosyltransferases type 1, 2 and 3. The approximate positions of RNA molecular weight markers are shown to the left. The expected size for the type 1 mRNA is 2.2 kb. The about 4.3 kb band in the type 1 blot may represent a partially processed RNA. The data indicates an absence of type 1 production in the magnum.

FIG. 3 shows the expression analysis of chicken sialyltransferases 1, 3, 4 and 6. mRNA was isolated from cultured fibroblasts (F), magnum (M), liver (L) and kidney (K) tissues of a laying hen and analyzed by the Northern blot method. Blots were probed with sequences complementary to the chicken 1, 3, 4 and 6 sialyltransferases. The approximate positions of RNA molecular weight markers are shown to the left. The data indicates low expression of type 3 and possibly low expression of type 4 in the magnum.

FIG. 4 shows a flow diagram of an exemplary two vector strategy. The GalT1 flock can be produced with the pALV-SIN-1.8-OM-GalT1 transgene shown in FIG. 6A. The SialT3 flock can be produced with the pALV-SIN-1.8-OM-SialT3 transgene shown in FIG. 6B. EW means egg white. Sial means sialic acid. Gal means galactose. GS birds are birds that contain transgenes for both GalT1 and SialT3. Gal/Sial means Gal and sialic acid. GGSS birds are birds homozygous for both GalT1 and SialT3 transgenes. A “protein production flock” is a flock that produces a protein with attached oligosaccharide structure(s) such as a therapeutic protein the effectiveness of which can be enhanced by the addition of Gal and/or sialic acid to the oligosaccharide structure(s).

FIG. 5 shows a flow diagram of an exemplary one vector strategy. The flock is produced with the pALV-SIN-1.8-OM-GalT1-IRES-SialT3 vector shown in FIG. 6C. EW means egg white.

FIGS. 6A, B and C show maps of pALV-SIN-1.8-OM-GalT1, pALV-SIN-1.8-OM-SialT3 and pALV-SIN-1.8-OM-GalT1-IRES-SialT3 vectors respectively. The retroviral transgene portion of each vector is shown. The vector backbones are not shown for simplicity. Upon integration of the vector in the chick embryo cells, the 3′ SIN LTR is copied over to the 5′ LTR such that the transgene is flanked by inactivated LTRs. FIG. 6A shows a 1.8 kb ovomucoid promoter operably linked to the chicken beta-1,4-galactosyltransferase type 1 coding sequence. FIG. 6B shows a 1.8 kb ovomucoid promoter operably linked to the chicken alpha-2,3-sialyltransferase type 3. FIG. 6C shows a 1.8 kb ovomucoid promoter operably linked to the chicken beta-1,4-galactosyltransferase type 1 coding sequence and chicken alpha-2,3-sialyltransferase 3 coding sequence with an IRES between the two coding sequences such as the translational enhancer disclosed in U.S. Pat. No. 4,937,190, issued Jun. 26, 1990, the disclosure of which is incorporated in its entirety herein by reference.

FIG. 7 shows a general method and timeline for producing transgene-augmented glycosylation birds.

FIG. 8A shows a MALDI-MS analysis of oligosaccharide structures of egg white proteins produced in a transgene-augmented glycosylation chicken having the GalT1 transgene incorporated into its genome using the vector shown in FIG. 6A (FIG. 9). Thirteen separate analyses were performed and the figure shows exemplary results of one of the runs. FIG. 8B-8C shows additional oligosaccharide structures that have Gal and/or sialic acid added that were also identified in one or more of the other twelve analyses (mass/mz is specified for each). FIG. 8D is a control sample. The data demonstrates that Gal and some Sialic Acid were added to oligosaccharide structures present on egg white protein as a result of transgene-augmented glycosylation. Legend: =mannose; ▴=fucose; ∘=galactose; ▪=N-acetylglucosamine; ♦=sialic acid.

The invention includes proteins having an N-linked oligosaccharide, for example, human proteins including those disclosed in this application (e.g., human proteins) which can be expressed in the oviduct of transgene augmented glycosylation birds having novel oligosaccharide structures.

FIG. 9A-C (SEQ ID NO: 1) shows pSIN-OM-1.8-GalT1 which is 7434 bp in length. Some features of the sequence are as follows: LTR-nucleotides 370 . . . 542; LTR-3645 . . . 3990; CDS-268 . . . 7356; promoter 4441 . . . 6214.

FIG. 10A-C (SEQ ID NO: 2) shows pSIN-OM-1.8-SialT3 which is 7545 bp in length. Some features of the sequence are as follows: LTR-nucleotides 370 . . . 542; LTR-3645 . . . 3990; CDS-6362 . . . 7540 promoter 4431 . . . 6309.

FIG. 11A-C (SEQ ID NO: 3) shows pSIN-OM-1.8-GalT1-IRES-SialT3 9119 bp in length. Some features of the sequence are as follows: LTR-nucleotides 3653 . . . 3998; LTR-nucleotides 378 . . . 550; CDS-nucleotides 7930 . . . 9108; CDS-nucleotides 6276 . . . 7361; promoter nucleotides 4449 . . . 6222; IRES 7362 . . . 7929. It is contemplated that one or more of the following nucleotide substitutions will enhance the quantity of translated product produced by the IRES: nt 7920 T to G; nt 7918 C to A; nt 7917 G to T; nt 7836 G to A; nts 7366 to 7368 (CCC) replaced with AATTCCCCCTCTCCCTCCCCCCCCCCTAAC (SEQ ID NO: 39).

FIG. 12A (SEQ ID NO: 4) shows chicken beta-1,4-galactosyltransferase (CKI) mRNA type 1-ACCESSION NO. U19890. Some of the features are: 5′UTR-nucleotides 1 . . . 57; CDS-nucleotides 58 . . . 1146; 3′UTR-nucleotides 1147.2279; polyA signal-nucleotides 2260 . . . 2265

FIG. 12B (SEQ ID NO: 5) shows the amino acid sequence for chicken beta-1,4-galactosyltransferase.

FIG. 13A (SEQ ID NO: 6) shows chicken beta-1,4-galactosyltransferase (CKII) mRNA type 2-ACCESSION U19889. The CDS is shown by nucleotides 202 . . . 1323.

FIG. 13B (SEQ ID NO: 7) shows the amino acid sequence for chicken beta-1,4-galactosyltransferase.

FIG. 14A (SEQ ID NO: 8) shows chicken beta-1,4-galactosyltransferase, type 3 mRNA-ACCESSION NO: XM_{—}416564. The CDS is shown by nucleotides 1 . . . 1029.

FIG. 14B (SEQ ID NO: 9) shows the amino acid sequence for chicken beta-1,4-galactosyltransferase, type 3.

FIG. 15A (SEQ ID NO: 10) shows chicken beta-1,4-galactosyltransferase, type 4 mRNA-ACCESSION XM_{—}416563. The CDS is shown by nucleotides 221 . . . 1288.

FIG. 15B (SEQ ID NO: 11) shows the amino acid sequence for chicken beta-1,4-galactosyltransferase, type 4.

FIG. 16A (SEQ ID NO: 12) shows chicken beta-1,4-galactosyltransferase, type 5 mRNA. The CDS is shown by nucleotides 1 . . . 1773.

FIG. 16B (SEQ ID NO: 13) shows the amino acid sequence for chicken beta-1,4-galactosyltransferase, type 5.

FIG. 17A-B (SEQ ID NO: 14) shows chicken beta-1,4-galactosyltransferase, type 6 mRNA. The CDS is shown by nucleotides 294 . . . 1400.

FIG. 17C (SEQ ID NO: 15) shows the amino acid sequence for chicken beta-1,4-galactosyltransferase, type 6.

FIG. 18A (SEQ ID NO: 16) shows chicken beta-1,4-galactosyltransferase, type 7 mRNA. The CDS is shown by nucleotides 57 . . . 1016.

FIG. 18B (SEQ ID NO: 17) shows the amino acid sequence for chicken beta-1,4-galactosyltransferase, type 7.

FIG. 19A (SEQ ID NO: 18) shows chicken alpha-2,3-sialyltransferase 1 mRNA. The CDS is shown by nucleotides 132 . . . 1160.

FIG. 19B (SEQ ID NO: 19) shows the amino acid sequence for chicken alpha-2,3-sialyltransferase 1.

FIG. 20A (SEQ ID NO: 20) shows chicken alpha-2,3-sialyltransferase 2 mRNA. The CDS is shown by nucleotides 290 . . . 1339.

FIG. 20B (SEQ ID NO: 21) shows the amino acid sequence for chicken alpha-2,3-sialyltransferase 2.

FIG. 21A (SEQ ID NO: 22) shows chicken alpha-2,3-sialyltransferase 3 mRNA. The CDS is shown by nucleotides 1 . . . 1179.

FIG. 21B (SEQ ID NO: 23) shows the amino acid sequence for chicken alpha-2,3-sialyltransferase 3.

FIG. 21C (SEQ ID NO: 24) shows an alternate isoform having an amino acid sequence segment deleted, i.e., the corresponding nucleotide sequence segment is deleted in the nucleotide sequence shown in FIG. 21a.

FIG. 22A (SEQ ID NO: 25) shows chicken alpha-2,3-sialyltransferase 4 mRNA. The CDS is shown by nucleotides 325 . . . 1332.

FIG. 22B (SEQ ID NO: 26) shows the amino acid sequence for chicken alpha-2,3-sialyltransferase 4.

FIG. 23A (SEQ ID NO: 27) shows chicken alpha-2,3-sialyltransferase 5 mRNA. The CDS is shown by nucleotides 128 . . . 1234.

FIG. 23B (SEQ ID NO: 28) shows the amino acid sequence for chicken alpha-2,3-sialyltransferase 5.

FIG. 24A (SEQ ID NO: 29) shows chicken alpha-2,3-sialyltransferase 6 mRNA. The CDS is shown by nucleotides 740 . . . 1798.

FIG. 24B (SEQ ID NO: 30) shows the amino acid sequence for chicken alpha-2,3-sialyltransferase 6.

FIG. 25A (SEQ ID NO: 31) shows chicken alpha-2,6-sialyltransferase 1 mRNA. The CDS is shown by nucleotides 359 . . . 1600.

FIG. 25B (SEQ ID NO: 32) shows the amino acid sequence for chicken alpha-2,6-sialyltransferase 1.

FIG. 26A (SEQ ID NO: 33) shows chicken alpha-2,6-sialyltransferase 2 mRNA. The CDS is shown by nucleotides 1 . . . 1590.

FIG. 26B (SEQ ID NO: 34) shows the amino acid sequence for chicken alpha-2,6-sialyltransferase 2.

FIG. 27A (SEQ ID NO: 35) shows chicken alpha-2,6-sialyltransferase 4 mRNA. The CDS is shown by nucleotides. The CDS is shown by nucleotides 62 . . . 931.

FIG. 27B (SEQ ID NO: 36) shows the amino acid sequence for chicken alpha-2,6-sialyltransferase 4.

FIG. 28A (SEQ ID NO: 37) shows chicken alpha-2,6-sialyltransferase 5 mRNA. The CDS is shown by nucleotides 51 . . . 1100.

FIG. 28B (SEQ ID NO: 38) shows the amino acid sequence for chicken alpha-2,6-sialyltransferase 5.

### DETAILED DESCRIPTION

Some of the definitions and abbreviations used herein include the following: aa,

amino acid(s); bp, base pair(s); CDS, coding sequence cDNA, DNA complementary to an RNA; GalNac, N-acetylgalactosamine; Gal, galactose; GlcNac, IRES, internal ribosome entry site; N-acetylglucosamine nt, nucleotide(s); kb, 1000 base pairs; μg, microgram; ml, milliliter; ng, nanogram; nt, nucleotide.

Certain definitions are set forth herein to illustrate and define the meaning and scope of the various terms used to describe the invention herein.

The term “avian” as used herein refers to any species, subspecies or strain of organism of the taxonomic class ava, such as, but not limited to, such organisms as chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Ausstralorp, Minorca, Amrox, California Gray, Italian Partridge-colored), as well as strains of turkeys, pheasants, quails, duck, ostriches and other poultry commonly bred in commercial quantities.

The phrases “based on” and “derived from” typically mean obtained from, in whole or in part. For example, a retroviral vector being based on or derived from a particular retrovirus or based on a nucleotide sequence of a particular retrovirus mean that the genome of the retroviral vector contains a substantial portion of the nucleotide sequence of the genome of the particular retrovirus. The substantial portion may be a particular gene or nucleotide sequence such as the nucleotide sequence encoding the gag, pol and/or env proteins or other structural or functional nucleotide sequence of the virus genome such as sequences encoding the LTRs or may be substantially the complete retrovirus genome, for example, most (e.g., more than 60% or more than 70% or more than 80% or more than 90%) or all of the retrovirus genome, as will be apparent from the context in the specification as the knowledge of one skilled in the art. Examples of retroviral vectors that are based on or derived from a retrovirus are the NL retroviral vectors (e.g., NLB) which are based on the ALV retrovirus as disclosed in Cosset et al, Journal of Virology (1991) vol. 65, p 3388-3394.

The term “coding sequence” and “coding region” as used herein refer to nucleotide sequences and nucleic acid sequences, including both RNA and DNA, that encode genetic information for the synthesis of an RNA, a protein, or any portion of an RNA or protein.

Nucleotide sequences that are not naturally part of a particular organism's genome or are introduced at a non-native site in the organism's genome are referred to as “foreign” nucleotide sequences, “heterologous” nucleotide sequences, “recombinant” nucleotide sequences or “exogenous” nucleotide sequences. In addition, a nucleotide sequence that has been isolated and then reintroduced into the same type (e.g., same species) of organism is not considered to be a naturally occurring part of a particular organism's genome and is therefore considered exogenous or heterologous. “Heterologous proteins” or “exogenous proteins” can be proteins encoded by foreign, heterologous or exogenous nucleotide sequences and therefore are often not naturally expressed in a cell of the organism.

As used herein, the terms “exogenous”, “heterologous” and “foreign” with reference to nucleic acids, such as DNA and RNA, are used interchangeably and refer to nucleic acid that does not occur naturally as part of a chromosome, a genome or cell in which it is present or which is found in a location(s) and/or in amounts that differ from the location(s) and/or amounts in which it occurs in nature. It can be nucleic acid that is not endogenous to the genome, chromosome or cell and has been exogenously introduced into the genome, chromosome or cell. Examples of heterologous DNA include, but are not limited to, DNA that encodes a gene product or gene product(s) of interest, for example, for production of an encoded protein. Examples of heterologous DNA include, but are not limited to, DNA that encodes traceable marker proteins, DNA that encodes therapeutic proteins. The terms “heterologous” and “exogenous” can refer to a biomolecule such as a nucleic acid or a protein which is not normally found in a certain cell, tissue or substance produced by an organism or is not normally found in a certain cell, tissue or substance produced by an organism in an amount or location the same as that found to occur naturally. For example, a protein that is heterologous or exogenous to an egg is a protein that is not normally found in the egg.

The term “construct” as used herein refers to a linear or circular nucleotide sequence such as DNA that has been assembled from more than one segments of nucleotide sequence which have been isolated from a natural source or have been chemically synthesized, or combinations thereof.

The term “complementary” as used herein refers to two nucleic acid molecules that can form specific interactions with one another. In the specific interactions, an adenine base within one strand of a nucleic acid can form two hydrogen bonds with thymine within a second nucleic acid strand when the two nucleic acid strands are in opposing polarities. Also in the specific interactions, a guanine base within one strand of a nucleic acid can form three hydrogen bonds with cytosine within a second nucleic acid strand when the two nucleic acid strands are in opposing polarities. Complementary nucleic acids as referred to herein, may further comprise modified bases wherein a modified adenine may form hydrogen bonds with a thymine or modified thymine, and a modified cytosine may form hydrogen bonds with a guanine or a modified guanine.

The term “cytokine” as used herein refers to any secreted amino acid sequence that affects the functions of cells and is a molecule that modulates interactions between cells in the immune, inflammatory or hematopoietic responses. A cytokine includes, but is not limited to, monokines and lymphokines regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and b-lymphocytes. Lymphokines are generally referred to as being produced by lymphocyte cells. Examples of cytokines include, but are not limited to, interferon, erythropoietin, G-CSF, Interleukin-1 (IL-1), Interleukin-6 (IL-6), Interleukin-8 (IL-8), Tumor Necrosis Factor-alpha (TNF-alpha) and Tumor Necrosis Factor beta (TNF-beta).

The term “expressed” or “expression” as used herein refers to the transcription of a coding sequence to yield an RNA nucleic acid molecule at least complementary in part to a region of one of the two nucleic acid strands of the coding sequence. The term “expressed” or “expression” as used herein can also refer to the translation of RNA to produce a protein or peptide.

The term “expression vector” as used herein refers to a nucleic acid vector that comprises a gene expression controlling region, such as a promoter or promoter component, operably linked to a nucleotide sequence encoding at least one polypeptide.

The term “fragment” as used herein can refer to, for example, an at least about 10, 20, 50, 75, 100, 150, 200, 250, 300, 500, 1000, 2000, 5000, 6,000, 8,000, 10,000, 20,000, 30,000, 40,000, 50,000 or 60,000 nucleotide long portion of a nucleic acid that has been constructed artificially (e.g., by chemical synthesis) or by cleaving a natural product into multiple pieces, using restriction endonucleases or mechanical shearing, or enzymatically, for example, by PCR or any other polymerizing technique known in the art, or expressed in a host cell by recombinant nucleic acid technology known to one of skill in the art. The term “fragment” as used herein may also refer to, for example, an at least about 5, 10, 20, 30, 40, 50, 75, 100, 150, 200, 250, 300, 400, 500, 1000, 2000, 5000, 6,000, 8,000 or 10,000 amino acid portion of an amino acid sequence, which portion is cleaved from a naturally occurring amino acid sequence by proteolytic cleavage by at least one protease, or is a portion of the naturally occurring amino acid sequence synthesized by chemical methods or using recombinant DNA technology (e.g., expressed from a portion of the nucleotide sequence encoding the naturally occurring amino acid sequence) known to one of skill in the art. “Fragment” may also refer to a portion, for example, of about 5%, about 10%, about 20%, about 30%, about 40%, about 50%, about 60%, about 70%, about 80% about 90% about 95% or about 99% of a particular nucleotide sequence or amino acid sequence.

“Functional portion” and “functional fragment” can be used interchangeably and as used herein mean a portion or fragment of a whole capable of performing, in whole or in part, a function of the whole. For example, a biologically functional portion of a molecule means a portion of the molecule that performs a biological function of the whole or intact molecule. Functional portions may be of any useful size. For example, a functional fragment may range in size from about 20 bases in length to a length equal to the entire length of the specified sequence minus one nucleotide. In another example, a functional fragment may range in size from about 50 bases in length to a length equal to the entire length of the specified sequence minus one nucleotide. In another example, a functional fragment may range in size from about 50 bases in length to about 20 kb in length. In another example, a functional fragment may range in size from about 500 bases in length to about 20 kb in length. In another example, a functional fragment may range in size from about 1 kb in length to about 20 kb in length. In another example, a functional fragment may range in size from about 0.1 kb in length to about 10 kb in length. In another example, a functional fragment may range in size from about 20 bases kb in length to about 10 kb in length.

The term “fully transgenic” refers to an animal such as a bird that contains at least one copy of a transgene in essentially all of its somatic cells.

The term “gene expression controlling region” as used herein refers to nucleotide sequences that are associated with a coding sequence and which regulate, in whole or in part, expression of the coding sequence, for example, regulate, in whole or in part, the transcription of the coding sequence. Gene expression controlling regions may be isolated from a naturally occurring source or may be chemically synthesized and can be incorporated into a nucleic acid vector to enable regulated transcription in appropriate cells. The “gene expression controlling regions” may precede, but is not limited to preceding, the region of a nucleic acid sequence that is in the region 5′ of the end of a coding sequence that may be transcribed into mRNA.

The term “isolated nucleic acid” as used herein covers, for example, (a) a DNA which has the sequence of part of a naturally occurring genomic molecule but is not flanked by at least one of the sequences that flank that part of the molecule in the genome of the species in which it naturally occurs; (b) a nucleic acid which has been incorporated into a vector or into the genomic DNA of a prokaryote or eukaryote in a manner such that the resulting vector or genomic DNA is not identical to naturally occurring DNA from which the nucleic acid was obtained; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, i.e., a gene encoding a fusion protein, and (e) a recombinant nucleotide sequence that is part of a hybrid sequence that is not naturally occurring. Isolated nucleic acid molecules of the present invention can include, for example, natural allelic variants as well as nucleic acid molecules modified by nucleotide deletions, insertions, inversions, or substitutions.

The term “nucleic acid” as used herein refers to any linear or sequential array of nucleotides and nucleosides, for example cDNA, genomic DNA, mRNA, tRNA, oligonucleotides, oligonucleosides and derivatives thereof. For ease of discussion, non-naturally occurring nucleic acids may be referred to herein as constructs. Nucleic acids can include bacterial plasmid vectors including expression, cloning, cosmid and transformation vectors such as, animal viral vectors such as, but not limited to, modified adenovirus, influenza virus, polio virus, pox virus, retroviruses such as avian leukosis virus (ALV) retroviral vector, a murine leukemia virus (MLV) retroviral vector, and a lentivirus vector, and the like and fragments thereof. In addition, the nucleic acid can be an LTR of an avian leukosis virus (ALV) retroviral vector, a murine leukemia virus (MLV) retroviral vector, or a lentivirus vector and fragments thereof. Nucleic acids can also include NL vectors such as NLB, NLD and NLA and fragments thereof and synthetic oligonucleotides such as chemically synthesized DNA or RNA. Nucleic acids can include modified or derivatized nucleotides and nucleosides such as, but not limited to, halogenated nucleotides such as, but not only, 5-bromouracil, and derivatized nucleotides such as biotin-labeled nucleotides.

The term “vector” and “nucleic acid vector” as used herein refers to a natural or synthetic single or double stranded plasmid or viral nucleic acid molecule that can be transfected or transformed into cells and replicate independently of, or within, the host cell genome. A circular double stranded vector can be linearized by treatment with an appropriate restriction enzyme based on the nucleotide sequence of the vector. A nucleic acid can be inserted into a vector by cutting the vector with restriction enzymes and ligating the desired pieces together, as is understood in the art.

The term “operably linked” refers to an arrangement of elements wherein the components so described are configured so as to perform their usual function. Gene expression controlling regions or promoters (e.g., promoter components) operably linked to a coding sequence are capable of effecting the expression of the coding sequence. The controlling sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “oviduct” or “oviduct tissue” refers to tissue of an avian oviduct, such as the magnum, e.g., tubular gland cells, where proteins are produced containing N-linked oligosaccharides that contain substantially reduced amounts of Gal and/or sialic acid relative to that of proteins produced in other tissue of the avian such as liver or kidney tissue.

The term “oviduct specific promoter” as used herein refers to promoters and promoter components which are functional, i.e., provide for transcription of a coding sequence, to a large extent, for example, primarily (i.e., more than 50% of the transcription product produced in the animal by a particular promoter type being produced in oviduct cells) or exclusively in oviduct cells of a bird. Examples of oviduct specific promoters include, ovalbumin promoter, ovomucoid promoter, ovoinhibitor promoter, lysozyme promoter and ovotransferrin promoter and functional portions of these promoters, e.g., promoter components. Glycosylation enzymes such as GalTs (e.g., GalT1) and SialTs (e.g., SialT3) are normally directed to the ER/Golgi organelles and participate in the N-glycan synthesis pathway. By limiting the expression of these enzymes to the magnum using oviduct specific promoters, deleterious physiological effects to the bird as result of expression of these enzymes in other tissues of the bird are minimized.

The terms “percent sequence identity”, “percent identity”, “% identity”, “percent sequence homology”, “percent homology”, “% homology” and “percent sequence similarity” can each refer to the degree of sequence matching between two nucleic acid sequences or two amino acid sequences. Such sequence matching can be determined using the algorithm of Karlin & Attschul (1990) Proc. Natl. Acad. Sci. 87: 2264-2268, modified as in Karlin & Attschul (1993) Proc. Natl. Acad. Sci. 90: 5873-5877. Such an algorithm is incorporated into the NBLAST and XBLAST programs of Attschul et al. (1990) T. Mol. Biol. Q15: 403-410. BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference amino acid sequence. To obtain gapped alignments for comparison purposes, Gapped BLAST is utilized as described in Attschul et al. (1997) Nucl. Acids Res. 25: 3389-3402. When utilizing BLAST and Gapped BLAST programs, the default parameters of the respective programs (e.g. XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons.

The term “poultry derived” refers to a composition or substance produced by or obtained from poultry. “Poultry” refers to birds that can be kept as livestock, including but not limited to, chickens, duck, turkey, quail and ratites. For example, “poultry derived” may refer to chicken derived, turkey derived and/or quail derived. The term “avian derived” refers to a composition or substance produced by or obtained from an avian.

The terms “polynucleotide,” “oligonucleotide”, “nucleotide sequence” and “nucleic acid sequence” can be used interchangeably herein and include, but are not limited to, coding sequences, i.e., polynucleotide(s) or nucleic acid sequence(s) which are transcribed and translated into polypeptide in vitro or in vivo when placed under the control of appropriate regulatory or control sequences; controlling sequences, e.g., translational start and stop codons, promoter sequences, ribosome binding sites, polyadenylation signals, transcription factor binding sites, transcription termination sequences, upstream and downstream regulatory domains, enhancers, silencers, DNA sequences to which a transcription factor(s) binds and alters the activity of a gene's promoter either positively (induction) or negatively (repression) and the like. No limitation as to length or to synthetic origin are suggested by the terms described herein.

As used herein the terms “polypeptide” and “protein” refer to a polymer of amino acids, for example, three or more amino acids, in a serial array, linked through peptide bonds. The term “polypeptide” includes proteins, protein fragments, protein analogues, oligopeptides and the like. The term “polypeptides” includes polypeptides as defined above that are encoded by nucleic acids, produced through recombinant technology (e.g., isolated from a transgenic bird), or synthesized. The term “polypeptides” further contemplates polypeptides as defined above that include chemically modified amino acids or amino acids covalently or noncovalently linked to labeling ligands.

The term “promoter” as used herein refers to a DNA sequence useful to initiate transcription by an RNA polymerase in an avian cell. A “promoter component” is a DNA sequence that can, by itself or, in combination with other DNA sequences effect or facilitate transcription. Promoter components can be functional fragments of promoters. For example, an ovomucoid promoter component includes, without limitation, the about 1.8 kb, the about 3.9 kb and the about 10 kb ovomucoid promoters disclosed in U.S. application Ser. No. 11/649,543, published May 17, 2007, which is incorporated in its entirety herein by reference. “Promoter components” can also encompass rearranged gene expression controlling regions which function to initiate RNA transcription and hybrid DNA molecules composed of naturally occurring DNA sequences and/or synthetic DNA sequences which function to initiate RNA transcription.

The terms “recombinant nucleic acid” and “recombinant DNA” as used herein refer to combinations of at least two nucleic acid sequences that are not naturally found in a eukaryotic or prokaryotic cell. The nucleic acid sequences may include, but are not limited to, nucleic acid vectors, gene expression regulatory elements, origins of replication, suitable gene sequences that when expressed confer antibiotic resistance, protein-encoding sequences and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

As used herein, the term “regulatory” sequences or elements include promoters, enhancers, terminators, stop codons, and other elements that can control gene expression.

A “retrovirus”, “retroviral particle”, “transducing particle”, or “transduction particle” refers to a replication-defective or replication-competent virus capable of transducing non-viral DNA or RNA into a cell.

A “SIN vector” is a self-inactivating vector. In particular, a SIN vector is a retroviral vector having an altered genome such that upon integration into genomic DNA of the target cell (e.g., avian embryo cells) the 5′ LTR of the integrated retroviral vector will not function as a promoter. For example, a portion or all of the nucleotide sequence of the retroviral vector that results in the U3 region of the 5′ LTR of the retroviral vector once integrated may be deleted or altered in order to reduce or eliminate promoter activity of the 5′ LTR. In certain examples, deletion of the CAAT box and/or the TAATA box from U3 of the 5′ LTR can result in a SIN vector, as is understood in the art.

The term “sense strand” as used herein refers to a single stranded DNA molecule from a genomic DNA that can be transcribed into RNA and translated into the natural polypeptide product of the gene. The term “antisense strand” as used herein refers to the single strand DNA molecule of a genomic DNA that is complementary with the sense strand of the gene.

A “therapeutic protein” or “pharmaceutical protein” is a substance that, in whole or in part, makes up a drug. In particular, “therapeutic proteins” and “pharmaceutical proteins” include an amino acid sequence which in whole or in part makes up a drug.

The terms “transcription regulatory sequences” and “promoter components” as used herein refer to nucleotide which regulates the transcriptional expression of a coding sequence. Exemplary transcription regulatory sequences include enhancer elements, hormone response elements, steroid response elements, negative regulatory elements, and the like. The “transcription regulatory sequences” may be isolated and incorporated into a vector to enable regulated transcription in appropriate cells of portions of the vector DNA. The “transcription regulatory sequence” may precede, but is not limited to, the region of a nucleic acid sequence that is in the region 5′ of the end of a protein coding sequence that is transcribed into mRNA. Transcriptional regulatory sequences may also be located within a protein coding region, for example, in regions of a gene that are identified as “intron” regions.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with certain concentrations of salt, for example, but without limitation, a calcium or magnesium salt, or exposing the cells to an electric field, detergent, or liposome material, to render the host cell competent for the uptake of the nucleic acid molecules.

As used herein, a “transgenic animal” is any non-human animal, such as an avian species, including the chicken, in which one or more of the cells of the animal contain heterologous nucleic acid introduced by way of human intervention, such as by transgenic techniques known in the art (see, for example, US patent publication No. 2007/0243165, published Oct. 18, 2007 (now, U.S. Pat. No. 7,511,120), the disclosure of which is incorporated in its entirety herein by reference) including those disclosed herein. The nucleic acid is introduced into an animal, directly or indirectly by introduction into a cell (e.g., egg or embryo cell) by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animal, the transgene can cause cells to express a recombinant form of the target protein or polypeptide. The terms “chimeric animal” or “mosaic animal” are used herein to refer to animals in which a transgene is found, or in which the recombinant nucleotide sequence is expressed, in some but not all cells of the animal. A germ-line chimeric animal contains a transgene in its germ cells and can give rise to an offspring transgenic animal in which most or all cells of the offspring will contain the transgene.

As used herein, the term “transgene” means a nucleic acid sequence (encoding, for example, a human protein) that is partly or entirely heterologous, i.e., foreign, to the animal or cell into which it is introduced, or, is partly or entirely homologous to an endogenous gene of the transgenic animal or cell into which it is introduced, but which is designed to be inserted, or is inserted, into the animal or cell genome in such a way as to alter the genome of the organism into which it is inserted (e.g., it is inserted at a location which differs from that of the natural gene or its insertion results in a knockout).

Techniques useful for isolating and characterizing the nucleic acids and proteins of the present invention are well known to those of skill in the art and standard molecular biology and biochemical manuals may be consulted to select suitable protocols for use without undue experimentation. See, for example, Sambrook et al., 1989, “Molecular Cloning: A Laboratory Manual”, 2nd ed., Cold Spring Harbor, the content of which is herein incorporated by reference in its entirety.

Exogenous therapeutic proteins expressed and secreted in the oviduct and endogenous egg white proteins both have N-glycan structures that lack Gal and sialic acid. It has been discovered that this is a result of the glycosylation enzymatic pathway which is responsible for sugar modification of egg white proteins.

To date multiple proteins that are deposited in the egg white of transgenic hens have been produced including erythropoietin, interferon alpha and G-CSF. Avian derived G-CSF and avian derived interferon alpha are both proteins with only O-glycans and no N-glycans. Some O-glycan structures of these two proteins are similar to human O-glycans, with a high proportion of the structures synthesized to completion (Rapp, et al. Transgenic Res 12: 569-75, 2003). In addition, these proteins have been shown to have high stability as well as high efficacy and low immunological response in patients, all of which are expected of proteins having proper O-linked glycosylation (Patel, et al. Int J Clin Pharmacol Ther 45: 161-8, 2007).

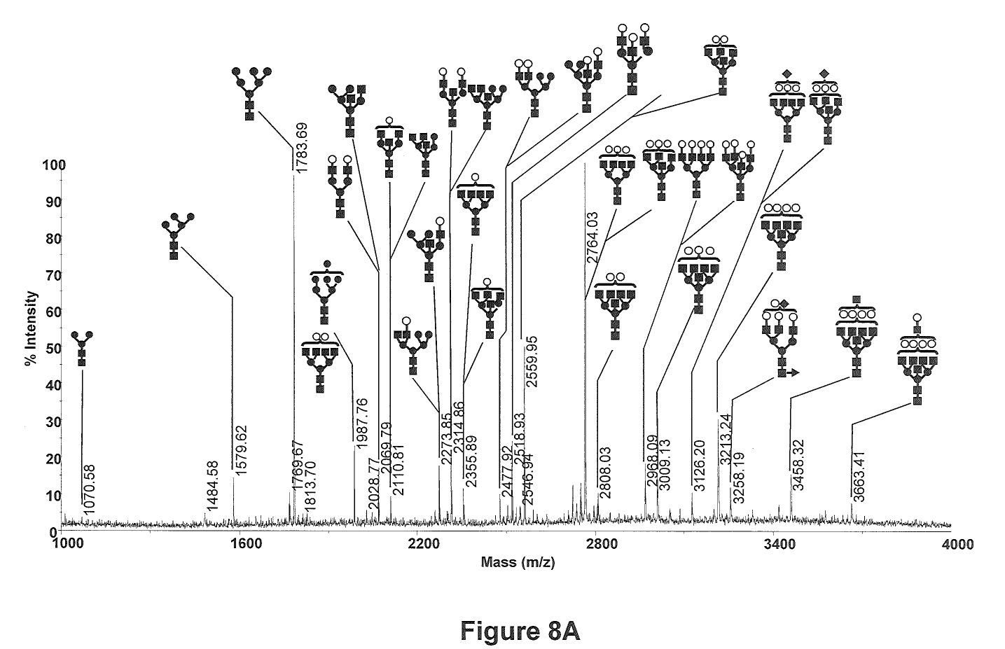

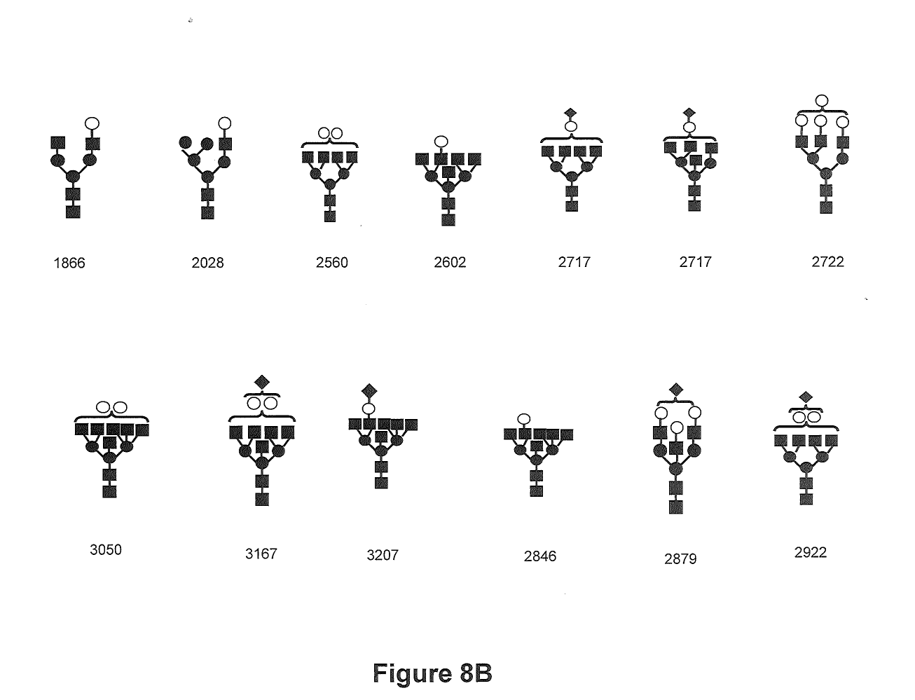

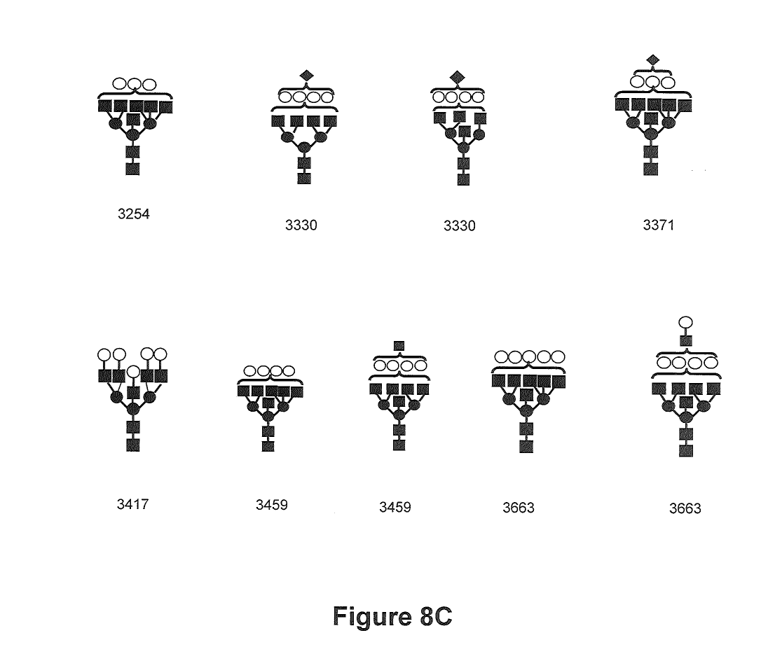

Glycosylation of proteins expressed in the hen oviduct that are modified with N-glycans such as human erythropoietin have been analyzed. See, for example, U.S. patent application Ser. No. 11/973,853, filed Oct. 10, 2007, the disclosure of which is incorporated in its entirety herein by reference. The basic N-linked structure has some similarities to that of human; however, there are also some differences. For example, in most cases of human N-glycans, a fucose is linked to the N-acetylglucosamine (GlcNac) residue that is linked to asparagine. In egg white proteins and recombinant proteins produced in the oviduct, this fucose is typically not present in large quantities. Human N-glycans are typically terminated in sialic acid at all or most terminal positions, being linked to galactose (Gal) which is linked to GlcNac. In the case of chicken egg white proteins and exogenous proteins produced in the oviduct, i.e., magnum (e.g., tubular gland cells), there is little or no sialic acid present in the N-glycans. In addition, there are typically few Gal residues at the terminal sugar in the N-glycan structures that have been characterized in transgenic avian derived exogenous protein (FIG. 8A). Thus, most of the terminal positions in N-glycans of egg white protein and exogenous protein produced and secreted by the chicken oviduct are occupied by GlcNac.

The inventor has also observed a bisecting GlcNac at the β1,4-linked mannose, a structure which is found in humans in certain tissues or cell types. Bisecting GlcNac is believed to increase the ADCC activity of antibodies. In one embodiment, transgenic avians of the invention have a transgene with a coding sequence for an acetylglucosaminyltransferase, such as N-acetylglucosaminyltransferase 3, linked to an oviduct specific promoter thereby imparting additional bisecting GlcNacs to N-linked oligosaccharide structures of protein produced in the oviduct (e.g., exogenous proteins such as antibodies).

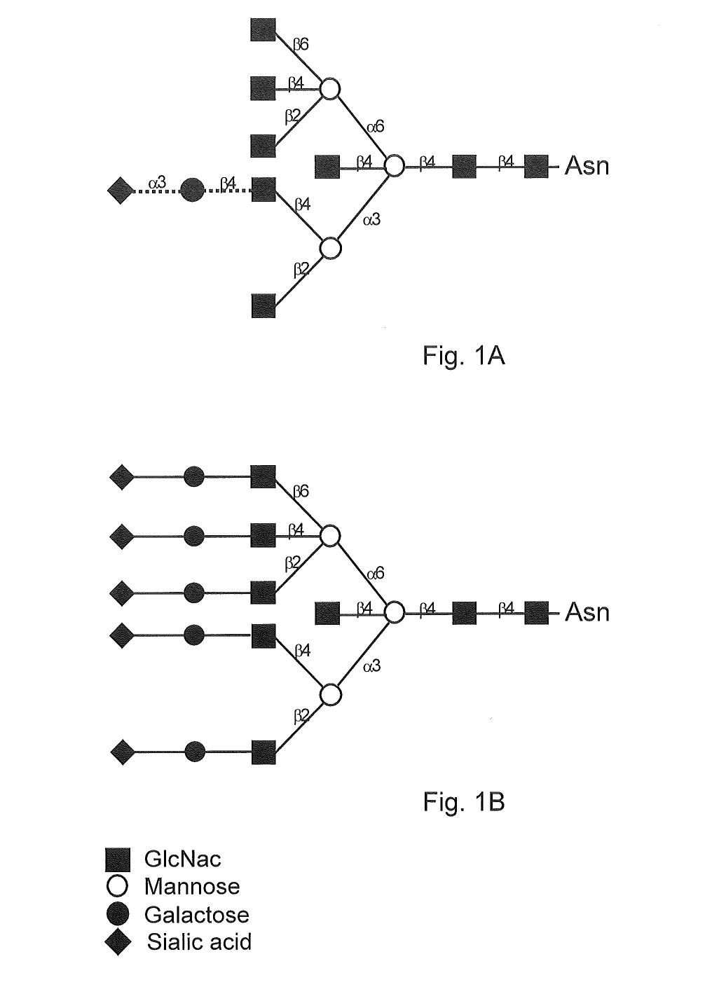

The N-glycans that reside on exogenous and endogenous (e.g., ovalbumin, ovomucoid) proteins produced and secreted in the oviduct have a basic structure essentially that shown in FIG. 1A see (Yamashita, et al. J Biol Chem 257: 12809-14, 1982; Harvey, et al. J Am Soc Mass Spectrom 11: 564-71, 2000; Lattova, et al. J Am Soc Mass Spectrom 15: 725-35, 2004) and U.S. patent application Ser. No. 11/973,853, filed Oct. 10, 2007.

In one embodiment, the present invention is directed to correcting the glycosylation deficiency by introduction into the avian genome transgenes that will express glycosyltransferases whose expression is deficient in the magnum, e.g., TGCs (tubular gland cells). Endogenous egg white proteins having N-glycans, such as ovalbumin and ovomucoid, can be harvested from eggs of transgenic hens and assessed for the presence of terminal sialic acid and/or terminal Gal and/or penultimate Gal resulting in transgene-augmented glycosylation flocks.

The transgene-augmented glycosylation flocks will have multiple uses. For example, a flock can be crossed to an existing flock that produces a therapeutic protein the effectiveness of which can be enhanced by an increase in the number of sialylated N-glycan structures. In another use, the transgenic flock can be used to produce entirely new production flocks having transgenes containing coding sequences for exogenous proteins which are expressed in the oviduct, e.g., magnum tissue. That is, an exogenous (e.g., therapeutic) protein transgene is introduced into a transgene-augmented glycosylation flock.

Chicken beta 1,4 GalT types 1 and 2 were previously identified by screening of a chicken hepatoma cDNA library (Shaper, N. L., J. A. Meurer, et al. (1997) J Biol Chem 272(50): 31389-99). Through analysis of the published chicken genome sequence the inventor has identified five additional GalTs (e.g., beta 1,4 GalT types 3 through seven) in the chicken genome that correspond to five members of the beta 1,4 GalT family which have been characterized in other species, including human, mouse, and hamster.

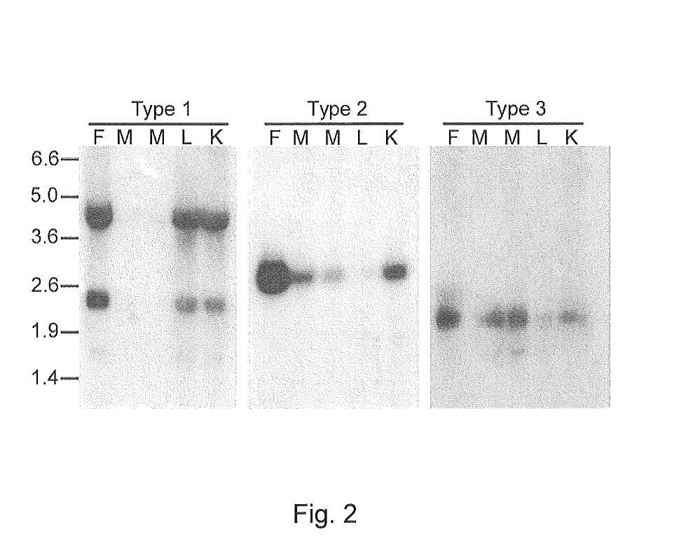

The expression of the seven chicken GalTs in several tissues, including magnum tissue, has been analyzed by Northern analysis. The expression of GalT1 was found to be almost undetectable in the magnum while it was expressed at detectable levels in cultured chicken fibroblasts as well as liver and kidney tissues, as shown in FIG. 2. Expression of type 6 was also not detectable in magnum tissues. Types 2 through 5 and 7 were all found to be expressed in the magnum tissues.

The lack of expression of GalT1 is a surprising result as GalT1 is thought to be ubiquitously expressed in a variety of tissues (Hennet. Cell Mol Life Sci 59: 1081-95, 2002). In other studies GalT1 was shown to be expressed in a number of chicken tissues, though magnum tissue expression was not assessed in those studies (Shaper, Meurer, Joziasse, Chou, Smith, Schnaar and Shaper. J Biol Chem 272: 31389-99, 1997).

The inventor has found that the lack of GalT1 expression in the magnum is responsible for a lack of N-linked Gal. GalT6 expression is also absent in the hen magnum. However, GalT6 is believed to be primarily responsible for the addition of Gal to glucose-ceramide, a step in the synthesis of the glycolipid lactosylceramide (Guo, et al. Glycobiology 11: 813-20, 2001) but not typically involved in addition of Gal to other proteins produced in the hen.

Therefore, in view of these discoveries, it is an object of the invention to produce transgenic birds which contain a transgene having a coding sequence for GalT1 operably linked to a promoter which can function in the oviduct resulting in the addition of Gal to N-linked oligosaccharides of protein produced in oviduct tissue as disclosed in the Examples. In addition, as expected these GalT1 birds also result in the addition of some sialic acid to N-linked oligosaccharides of the protein.

The expression of GalT1 provides for the addition of Gal to N-linked oligosaccharides produced in the magnum which can serve as a point of attachment for sialic acid. As can be seen in FIG. 8, additional sialic acid is added to the N-linked oligosaccharide structure of proteins produced in the magnum of GalT1 birds compared to normal birds.

It is also contemplated in accordance with the present invention, that deficiencies in expression of members of SialT family can be compensated for to provide for transgene-augmented glycosylation birds which allow for more sialation of N-linked oligosaccharides than in GalT1 birds.

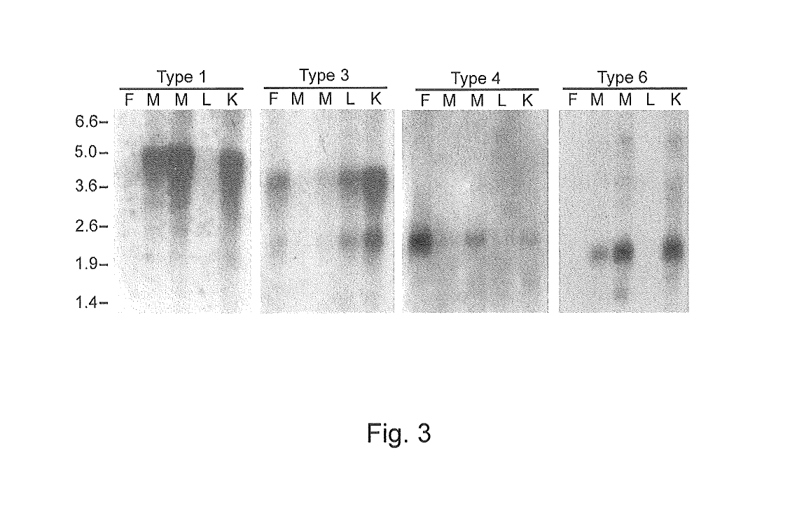

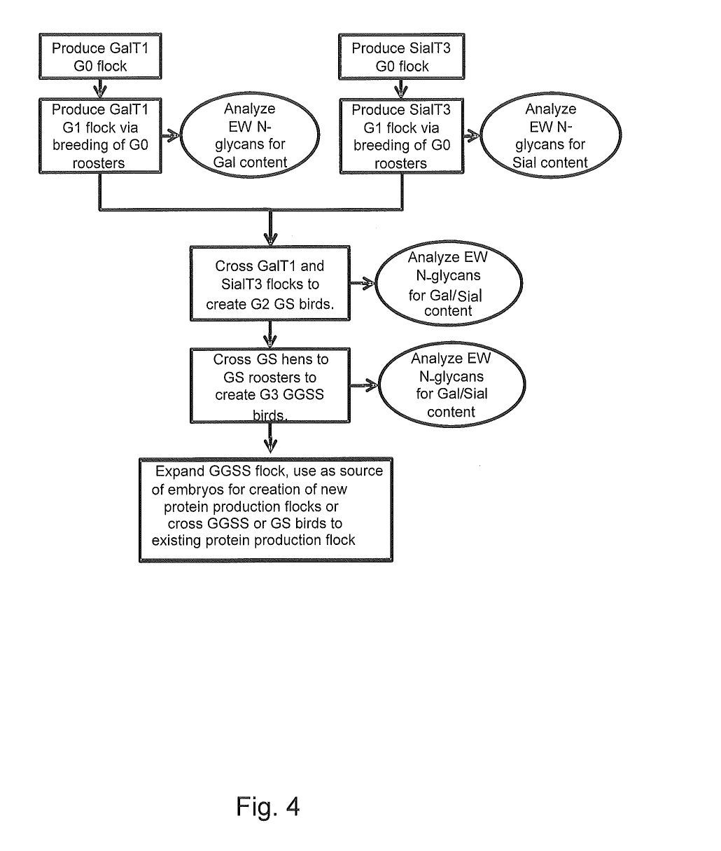

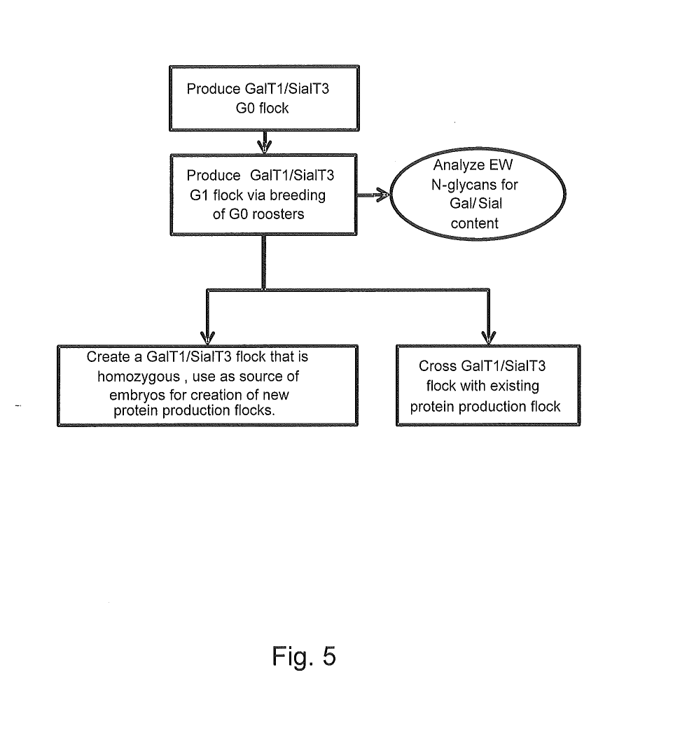

The inventor has analyzed the recently sequenced chicken genome finding that all six members of the α2,3 SialT family are present. Analysis of the expression of SialTs by the Northern blot method has also been performed. The expression of SialT1 in the magnum (FIG. 3) was very strong whereas the expression SialT2 was low, implicating SialT1 as having a major role in egg white O-glycan synthesis since the Galβ1,3GalNac chains in O-glycans found in egg white proteins are mostly sialylated (whereas the few Galβ1,4GlcNac chains present on N-glycans have little or no attached sialic acid). The expression of SialT3 in the magnum is detectable but is quite low relative to that of chicken fibroblast, kidney and liver (FIG. 3). The fact that SialT3 synthesis is fairly abundant in the kidney and liver and the N-glycans that arise from these organs are sialylated to a high degree (Ito, Takegawa, Deguchi, Nagai, Nakagawa, Shinohara and Nishimura. Rapid Commun Mass Spectrom 20: 3557-65, 2006; Deguchi, et al. Rapid Commun Mass Spectrom 20: 741-6, 2006; Sasaki, et al. J Biol Chem 262: 12059-76, 1987) indicates SialT3 may have a significant role in the sialylation of N-glycans in the chicken. A faint signal was detected for SialT4 in chicken fibroblasts and an even fainter signal in magnum and kidney. The low expression of SialT4 in the tissues examined suggests that SialT4 may have a lessor role in the sialylation of chicken N-glycans. The expression of SialT6 in the magnum and kidney is relatively high and undetectable in chicken fibroblasts and liver.

Therefore, in accordance with the invention, transgenic avians are contemplated that express one or more recombinant or exogenous SialT coding sequence(s) in the oviduct tissue, e.g., magnum tissue. In one embodiment, a transgenic avian, e.g., transgenic chicken, is contemplated that expresses in the magnum tissue (e.g., in tubular gland cells) an exogenous SialT coding sequence, i.e., a recombinant avian, e.g., chicken, nucleotide sequence that encodes a SialT. In one embodiment, a transgenic avian is contemplated that expresses an exogenous SialT1 coding sequence. In one embodiment, a transgenic avian is contemplated that expresses an exogenous SialT2 coding sequence. In one embodiment, a transgenic avian is contemplated that expresses an exogenous SialT3 coding sequence. In one embodiment, a transgenic avian is contemplated that expresses an exogenous SialT4 coding sequence. In one embodiment, a transgenic avian is contemplated that expresses an exogenous SialT5 coding sequence. In one embodiment, a transgenic avian is contemplated that expresses an exogenous SialT6 coding sequence.

In one particularly useful embodiment, a transgenic avian such as a chicken is produced that expresses a SialT3 coding sequence in its magnum tissue (e.g., in its tubular gland cells).

In one embodiment, a transgene-augmented glycosylation avian is produced in accordance with the invention that contains one or more transgenes that provides for expression of one or more GalTs in the oviduct and one or more transgenes that provide for expression of one or more SialTs in the oviduct. In a particularly useful but non-limiting example, a transgenic avian is produced in accordance with the invention that contains a transgene that provides for expression of GalT1 in the oviduct and a transgene that provides for expression of SialT3 in the oviduct. In another non-limiting example, a transgenic avian is produced in accordance with the invention that contains a transgene that provides for expression of GalT1 in the oviduct and a transgene that provides for expression of SialT3 in the oviduct and a transgene that provides for expression of SialT4 in the oviduct. In another non-limiting example, a transgenic avian is produced in accordance with the invention that contains a transgene that provides for expression of GalT1 in the oviduct and a transgene that provides for expression of GalT6 in the oviduct and a transgene that provides for expression of SialT3 in the oviduct and a transgene that provides for expression of SialT4 in the oviduct.

Many methods which are useful to express more than one (for example, two, three or four or more) exogenous nucleotide sequence in an avian genome are apparent to practitioners of ordinary skill in the art. For example, one such method which employs a single transcript containing an internal ribosome entry site (IRES) is described in Example 6. In another example, a fully transgenic bird (i.e., a G1 transgenic or a descendent of a G1 transgenic) containing a first desired transgene can have a second transgene introduced into its genome using standard methods. That is, the transgene can be introduced into the fully transgenic bird in essentially the same manner as the first transgene. In another example, the fully transgenic bird can be crossed to a second fully transgenic bird containing a desired transgene, as is understood in the art. These processes can be repeated to introduce the desired number of transgenes into the genome.

Any useful IRES is contemplated for use in accordance with the invention including those IRESs disclosed herein as well as any other useful IRESs (e.g., Foot an Mouth disease virus IRES, see for example, Belsham and Brangwyn (1990) J. of Virology, Vol. 64, p 5389-5395).

Any useful method can be used to introduce transgenes of the invention into an avian genome including, for example, those methods disclosed in US patent publication No. 2007/0180546 (now, U.S. Pat. No. 7,550,650), published Aug. 2, 2007; US patent publication No. 2007/0077650 (now, U.S. Pat. No. 7,524,626), published Apr. 5, 2007 and US patent publication No. 2008/0064862, published Mar. 13, 2008, the disclosures of each of these three patent applications are incorporated in their entireties herein by reference. One consideration is that the proteins produced in accordance with the methods disclosed in these cited documents were produced with a signal sequence so that the protein is secreted into the egg white whereas the glycosyltransferase produced in accordance with the present invention will not be secreted from the cell and therefore will typically not include a signal sequence.

Any gene expression controlling region (e.g., promoter) which can be made useful in accordance with the invention is contemplated for use in accordance with the invention. For example, constitutive promoters such as CMV and beta-actin which have been shown to function in the avian oviduct can be used. See, for example, US patent publication No. 2006/0015960, published Jan. 19, 2006 and 2006/0143725, published Jun. 29, 2006. The disclosure of each of these two patent applications is incorporated in its entirety herein by reference. In one particularly useful embodiment, the promoter is a promoter that primarily or exclusively is expressed in the oviduct such as ovomucoid promoters, ovalbumin promoters, and lysozyme promoters, conalbumin promoters, ovomucin promoters, ovotransferrin promoters. See, for example, US patent publication No. 2005/0176047, published, Aug. 11, 2005; U.S. Pat. No. 7,176,300, issued Feb. 13, 2007; US patent publication No. 2007/0124829, published May 31, 2007; and US patent publication No. 2006/0130170, published Jun. 15, 2006. The disclosure of each of these three patent applications and one issued patent is incorporated in its entirety herein by reference. Such promoters can be useful to avoid over expression of the glycosyltransferases in tissue aside from oviduct tissue of the avian, which could be problematic for the health or survival of the transgenic birds. Other promoters useful in accordance with the invention include, for example and without limitation, MDOT promoters and rous-sarcoma virus (RSV) promoters, murine leukemia virus (MLV) promoters, mouse mammary tumor virus (MMTV) promoters and SV40 promoters and functional portions of each of these promoters. Other promoters which may be useful in the present invention include, without limitation, Pol III promoters (for example, type 1, type 2 and type 3 Pol III promoters) such as H1 promoters, U6 promoters, tRNA promoters, RNase MPR promoters and functional portions of each of these promoters. Typically, functional terminator sequences are selected for use in the present invention in accordance with the promoter that is employed, as is understood in the art.

In one useful embodiment, a 1.8 kb ovomucoid promoter is employed which is disclosed essentially in US patent publication No. 2007/0113299, published May 17, 2007, the disclosure of which is incorporated in its entirety herein by reference. The 1.8 kb OM promoter has provided useful expression of GalT coding sequence in magnum cells as seen in Example 3. Other glycosylation enzymes are contemplated for production in the oviduct under the control of the 1.8 kb ovomucoid promoter.

Proteins which are contemplated for production in transgene-augmented glycosylation birds of the invention specifically include therapeutic proteins, including, but not limited to human proteins, which contain one or more N-linked oligosaccharide structures. Such proteins include, but are not limited to, the following proteins including where applicable their human protein equivalent: fusion proteins, growth hormones, cytokines, structural proteins and enzymes including human growth hormone, interferon, lysozyme, and β-casein, albumin, α-1 antitrypsin, antithrombin III, collagen, factors VIII, IX, X (and the like), fibrinogen, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), somatotropin, and chymotrypsin, glucocerebrosidase, lysosomal acid lipase, β-galactosidase and neuraminidase; galactosylceramidase (GALC); agalsidase alfa (Replagal), agalsidase beta (Fabrazyme) or α-galactosidase A; α-glucosidase; acid sphingomyelinase (rhASM); galactosylceramidase (GALC); modified immunoglobulins and antibodies, including immunotoxins which may bind to surface antigens on human tumor cells, b-domain deleted factor VIII, factor VIIa, anticoagulants; hirudin, alteplase, tPA, reteplase, tPA—3 of 5 domains deleted, insulin, insulin lispro, insulin aspart, insulin glargine, long-acting insulin analogs, hgh, glucagons, tsh, follitropin-beta, fsh, gm-csf, pdgh, IFN-α2, IFN-α2a, IFN-α2b, IFN-α, IFN-betα 1b, IFN-beta 1a, IFN-gamma1b, IL-2, IL-11, hbsag, ospa, mab directed against t-lymphocyte antigen, mAb directed against tag-72, tumor-associated glycoprotein, Fab fragments derived from chimeric mAb directed against platelet surface receptor gpII(b)/III(a), mAb or mAb fragment directed against tumor-associated antigen CA-125, mAb or mAb fragment directed against human carcinoembryonic antigen, cea, mAb or mAb fragment directed against human cardiac myosin, mAb or mAb fragment directed against tumor surface antigen psma, mAb fragments (Fab/Fab2 mix) directed against HMW-MAA, mAb or mAb fragment (Fab) directed against carcinoma-associated antigen, mAb fragments (Fab) directed against NCA 90, a surface granulocyte nonspecific cross reacting antigen, chimeric mAb directed against CD20 antigen found on surface of b-lymphocytes, humanized mAb directed against the alpha chain of the IL2 receptor, chimeric mAb directed against the alpha chain of the IL-2 receptor, chimeric mAb directed against TNF-α, humanized mAb directed against an epitope on the surface of respiratory synctial virus, humanized mAb directed against HER2, human epidermal growth factor receptor 2, human mAb directed against cytokeratin tumor-associated antigen anti-CTLA4, chimeric mAb directed against CD20 surface antigen of b-lymphocytes dornase-alpha, β-glucocerebrosidase, TNF-α, IL-2-diptheria toxin fusion protein, TNFR-IgG fragment fusion protein, laronidase, DNAase, mAbs, alefacept, tositumomab, alemtuzumab, rasburicase, agalsidase beta, teriparatide, parathyroid hormone derivatives, adalimumab (IgG1), anakinra, biological modifier, nesiritide, human b-type natriuretic peptide (HBNP), colony stimulating factors, pegvisomant, human growth hormone receptor antagonist, recombinant activated protein c, omalizumab, immunoglobulin E (IgE) blocker, lbritumomab, tiuxetan, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, etanercept, antidiuretic hormones, prolactin, thyroid stimulating hormone, multimeric proteins including immunoglobulins, such as antibodies, and antigen binding fragments thereof, an immunoglobulin heavy chain polypeptide comprising a variable region or a variant thereof, which may comprise a D region, a J region, a C region, or a combination thereof; an immunoglobulin light chain polypeptide comprising a variable region or a variant thereof which may comprise a J region and a C region; an immunoglobulin polypeptide encoded by at least one expression vector comprises an immunoglobulin heavy chain variable region, an immunoglobulin light chain variable region, and a linker peptide thereby forming a single-chain antibody capable of selectively binding an antigen; HERCEPTIN™ (Trastuzumab) (Genentech, Calif.) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath; Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD2O IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); CAT-152, a human anti-TGF-β₂ antibody (Cambridge Ab Tech); Cetuximab (BMS) is a monoclonal anti-EGF receptor (EGFr) antibody; Bevacizuma (Genentech) is an anti-VEGF human monoclonal antibody; Infliximab (Centocore, J&J) is a chimeric (mouse and human) monoclonal antibody used to treat autoimmune disorders; Gemtuzumab ozogamicin (Wyeth) is a monoclonal antibody used for chemotherapy; Ranibizumab (Genentech) is a chimeric (mouse and human) monoclonal antibody used to treat macular degeneration, GM-CSF, interferon β, fusion protein, CTLA4-Fc fusion protein, growth hormones, cytokines, structural, interferon, lysozyme, β-casein, albumin, α-1 antitrypsin, antithrombin III, collagen, factors VIII, IX, X (and the like), fibrinogen, lactoferrin, protein C, tissue-type plasminogen activator (tPA), somatotropin, and chymotrypsin, immunoglobulins, antibodies, immunotoxins, factor VIII, b-domain deleted factor VIII, factor VIIa, factor IX, anticoagulants, hirudin, alteplase, reteplase, tPA—3 of 5 domains deleted, insulin, insulin lispro, insulin aspart, insulin glargine, long-acting insulin analogs, glucagons, tsh, follitropin-β, fsh, PDGH, IFN-beta, IFN-beta 1, IFN-beta 2, IFN-α, IFN-α1, IFN-α2, IFN-gamma, IL-2, IL-11, hbsag, ospa, dornase alfa, β-glucocerebrosidase, TNF-α, IL-2-diptheria toxin fusion protein, TNFR-IgG fragment fusion protein, laronidase, DNAse, alefacept, tositumomab, murine mAb, alemtuzumab, rasburicase, agalsidase beta, teriparatide, parathyroid hormone derivatives, adalimumab (IgG1), anakinra, biological modifier, nesiritide, human b-type natriuretic peptide (HBNP), colony stimulating factors, pegvisomant, human growth hormone receptor antagonist, recombinant activated protein c, omalizumab, immunoglobulin E (IgE) blocker, lbritumomab, tiuxetan, ACTH, glucagon, somatostatin, somatotropin, thymosin, parathyroid hormone, pigmentary hormones, somatomedin, luteinizing hormone, chorionic gonadotropin, hypothalmic releasing factors, etanercept, antidiuretic hormones, prolactin and thyroid stimulating hormone, an immunoglobulin polypeptide, immunoglobulin polypeptide D region, immunoglobulin polypeptide J region, immunoglobulin polypeptide C region, immunoglobulin light chain, immunoglobulin heavy chain, an immunoglobulin heavy chain variable region, an immunoglobulin light chain variable region and a linker peptide.

Proteins such as those disclosed herein not normally N-glycosylated can be engineered to contain a glycosylation site (i.e., an N-linked glycosylation site) which is glycosylated in the avian system, as is understood by a practitioner of skill in the art. In addition, proteins such as those disclosed herein can be engineered to contain one or more additional N-linked glycosylation sites. In one embodiment, the protein with an added glycosylation site has attached one or more N-linked oligosaccharide structures with terminal modifications produced as disclosed herein.

It is specifically contemplated that proteins produced as disclosed herein can be isolated or purified using methodologies well known to practitioners of ordinary skill in the art.

In one embodiment, eggs laid by avians produced in accordance with the invention contain an exogenous or heterologous protein (such as a therapeutic protein) having an altered glycosylation pattern produced in the oviduct as disclosed herein in an amount greater than about 0.01 μg per hard-shell egg. For example, the eggs may contain the heterologous protein in an amount in a range of between about 0.01 μg per hard-shell egg and about 2 grams per hard-shell egg. In one embodiment, the eggs contain between about 0.1 μg per hard-shell egg and about 1 gram per hard-shell egg. For example, the eggs may contain between about 1 μg per hard-shell egg and about 1 gram per hard-shell egg. In one embodiment, the eggs contain between about 10 μg per hard-shell egg and about 1 gram per hard-shell egg. For example, the eggs may contain between about 100 μg per hard-shell egg and about 1 gram per hard-shell egg (e.g., the eggs may contain between about 100 μg per hard-shell egg and about 100 mg per hard-shell egg).

Typically, the heterologous protein (e.g., therapeutic protein) having an altered glycosylation pattern as disclosed herein is present in the egg white of the eggs. In one embodiment, the heterologous protein is present in egg white in an amount greater than about 0.01 μg per ml of the egg. In another embodiment, the heterologous protein is present in egg white in an amount in a range of between about 0.01 μg per ml of the egg white and about 0.2 gram per ml of the egg white. For example, the heterologous protein may be present in egg white in an amount in a range of between about 0.1 μg per ml of the egg white and about 0.5 gram per ml of the egg white. In one embodiment, the heterologous protein is present in egg white in an amount in a range of between about 1 μg per ml of the egg white and about 0.2 gram per ml of the egg white. For example, the heterologous protein may be present in egg white in an amount in a range of between about 10 μg per ml of the egg white and about 0.1 gram per ml of the egg white (e.g., the heterologous protein may be present in egg white in an amount in a range of between about 10 μg per ml of the egg white and about 5 mg per ml of the egg white).

The invention also contemplates that pegylating proteins produced as disclosed herein may be advantageous as discussed, for example, in U.S. patent application Ser. No. 11/584,832, filed Oct. 23, 2006, the disclosure of which is incorporated it its entirety herein by reference.

While it is possible that therapeutic proteins produced in accordance with this invention may be administered in raw form, it is preferable to administer the therapeutic proteins as part of a pharmaceutical formulation.

The invention thus further provides pharmaceutical formulations comprising therapeutic proteins produced in accordance with the invention or a pharmaceutically acceptable derivative thereof together with one or more pharmaceutically acceptable carriers thereof and, optionally, other therapeutic and/or prophylactic ingredients and methods of administering such pharmaceutical formulations. The carrier(s) must be “acceptable” in the sense of being compatible with the other ingredients of the formulation and not deleterious to the recipient thereof. Methods of treating a patient (e.g., quantity of pharmaceutical protein administered, frequency of administration and duration of treatment period) using pharmaceutical compositions of the invention can be determined using standard methodologies known to physicians of skill in the art.

Pharmaceutical formulations include those suitable for oral, rectal, nasal, topical (including buccal and sub-lingual), vaginal administration. The pharmaceutical formulations include those suitable for administration by injection including intramuscular, sub-cutaneous and intravenous administration. The pharmaceutical formulations also include those for administration by inhalation or insufflation. The formulations may, where appropriate, be conveniently presented in discrete dosage units and may be prepared by any of the methods well known in the art of pharmacy. The methods of producing the pharmaceutical formulations typically include the step of bringing the therapeutic proteins into association with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution; as a suspension; or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles, which may include edible oils, or preservatives.

Therapeutic proteins of the invention formulated for parenteral administration (e.g., by injection, for example bolus injection or continuous infusion) may be presented in unit dose form in ampoules, pre-filled syringes, small volume infusion or in multi-dose containers with an added preservative. The therapeutic proteins may be injected by, for example, subcutaneous injections, intramuscular injections, and intravenous infusions or injections.

The therapeutic proteins may take such forms as suspensions, solutions, or emulsions in oily or aqueous vehicles, and may contain formulatory agents such as suspending, stabilizing and/or dispersing agents. It is also contemplated that the therapeutic proteins may be in powder form, obtained by aseptic isolation of sterile solid or by lyophilization from solution, for constitution with a suitable vehicle, e.g., sterile, pyrogen-free water, before use.

For topical administration to the epidermis, the therapeutic proteins produced according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents or coloring agents.

Formulations suitable for topical administration in the mouth include lozenges comprising active ingredient in a flavored base, such as sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably represented as unit dose suppositories. Suitable carriers include cocoa butter and other materials commonly used in the art, and the suppositories may be conveniently formed by a mixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

For intra-nasal administration the therapeutic proteins of the invention may be used as a liquid spray or dispersible powder or in the form of drops.

Drops may be formulated with an aqueous or non-aqueous base also comprising one or more dispersing agents, solubilizing agents or suspending agents. Liquid sprays are conveniently delivered from pressurized packs.

For administration by inhalation, therapeutic proteins according to the invention may be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

For administration by inhalation or insufflation, the therapeutic proteins according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient, may be employed.

The pharmaceutical compositions according to the invention may also contain other active ingredients such as antimicrobial agents, or preservatives. In addition, it is contemplated that the therapeutic proteins of the invention may be used in combination with other therapeutic agents.

Compositions or compounds of the invention can be used to treat a variety of conditions. For example, there are many conditions for which treatment therapies are known to practitioners of skill in the art in which therapeutic proteins obtained from cell culture (e.g., CHO cells) are employed. The present invention contemplates that the therapeutic proteins produced in accordance with the invention can be employed to treat such conditions. That is, the invention contemplates the treatment of conditions known to be treatable by conventionally produced therapeutic proteins by using therapeutic proteins produced in accordance with the invention. For example, erythropoietin produced in accordance with the invention can be used to treat human conditions such as anemia and kidney disease, e.g., chronic renal failure (or other conditions which may be treatable by administering EPO of the invention).

Generally, the dosage administered will vary depending upon known factors such as age, health and weight of the recipient, type of concurrent treatment, frequency of treatment, and the like. Usually, a dosage of active ingredient can be between about 0.0001 and about 10 milligrams per kilogram of body weight. Precise dosage, frequency of administration and time span of treatment can be determined by a physician skilled in the art of administration of the respective therapeutic protein.

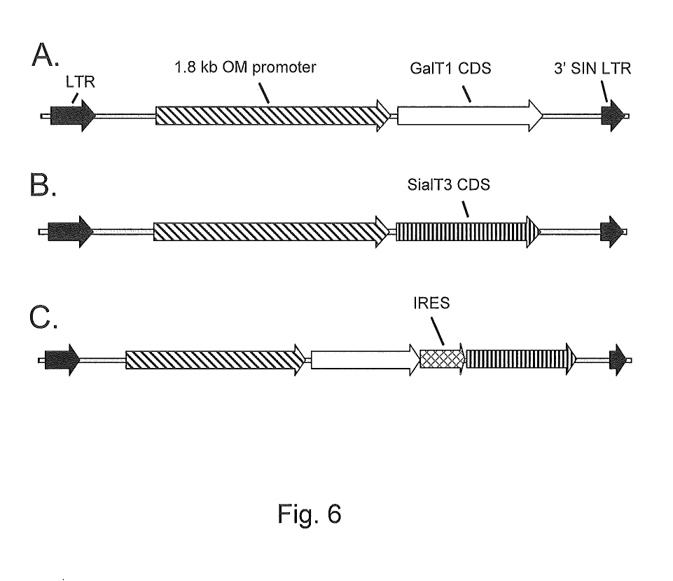

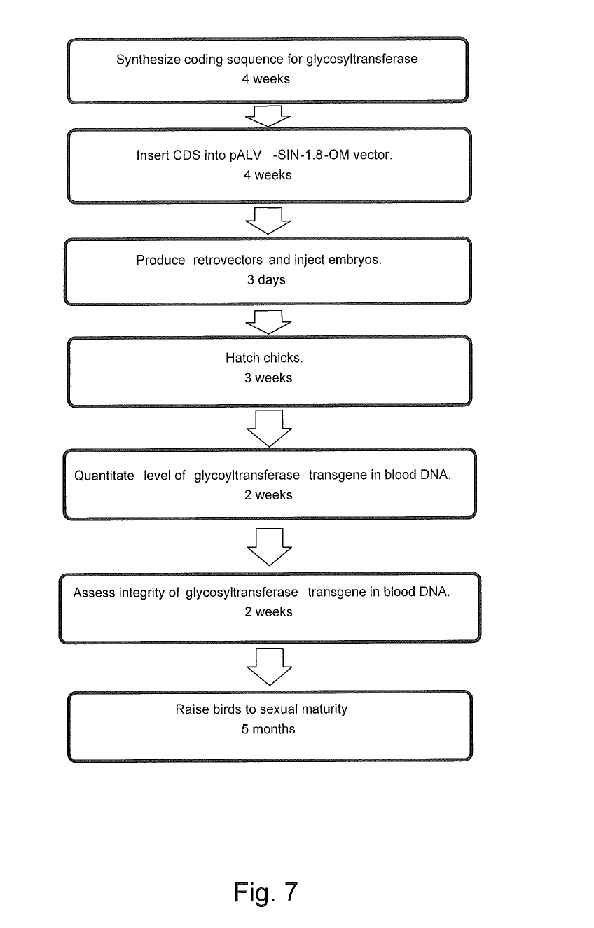

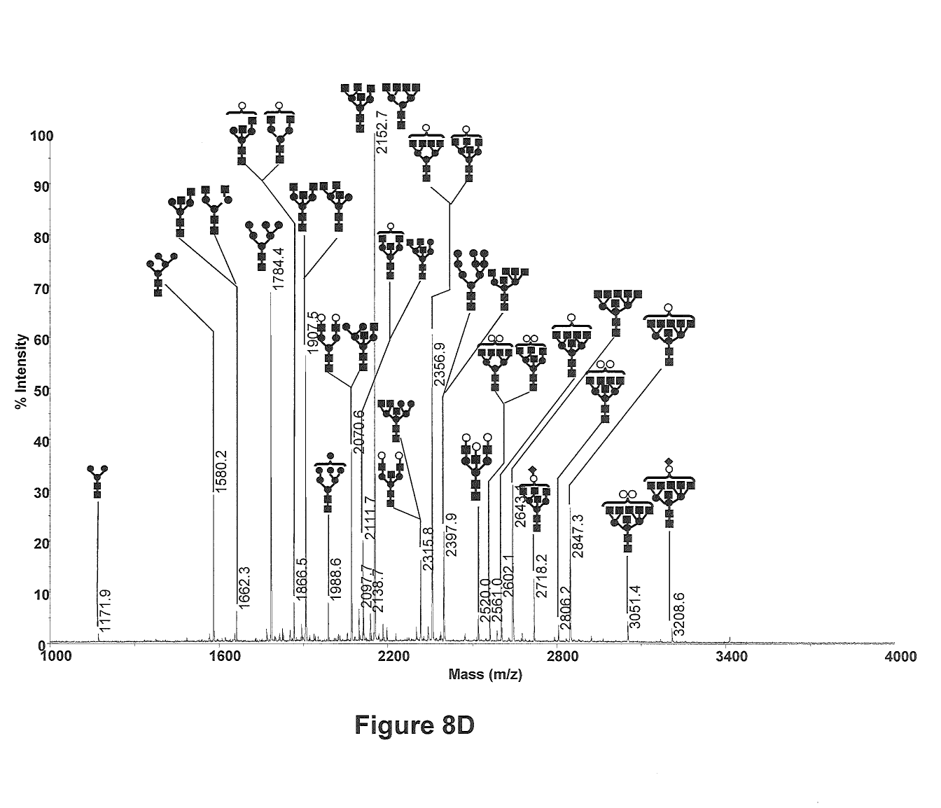

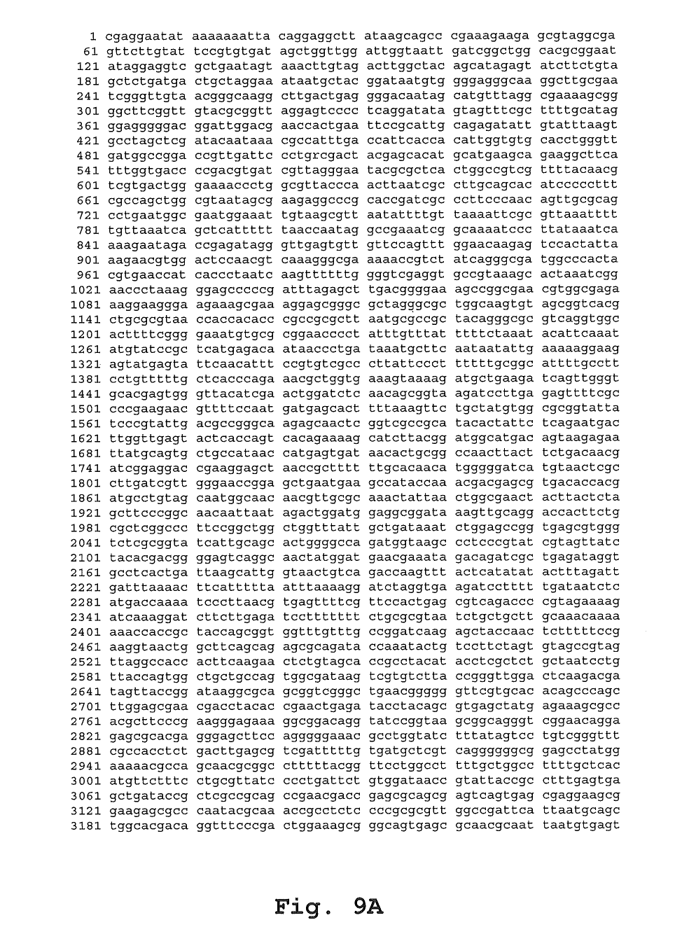

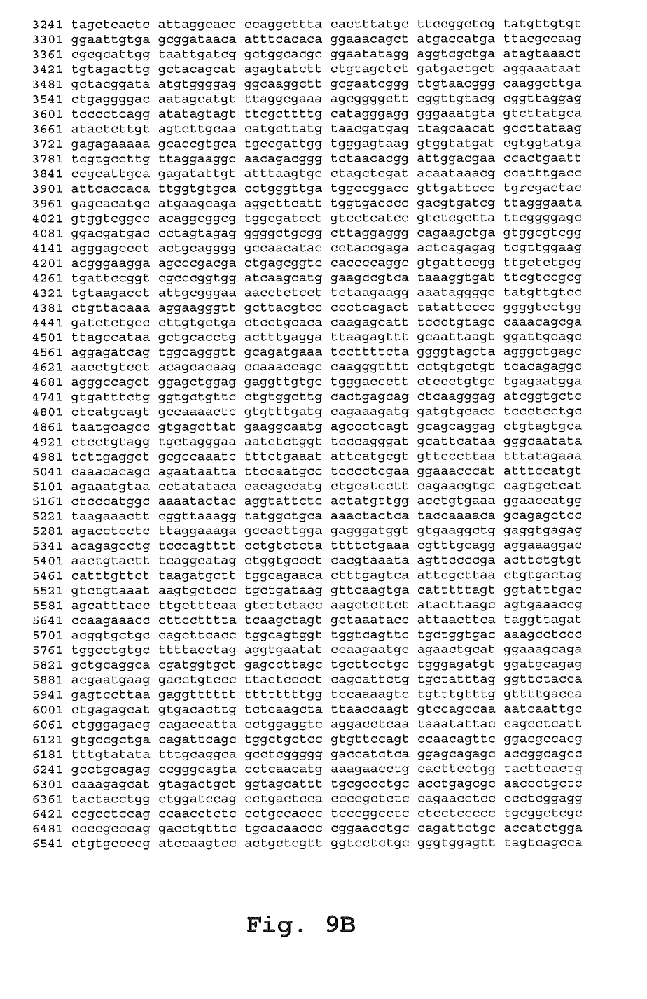

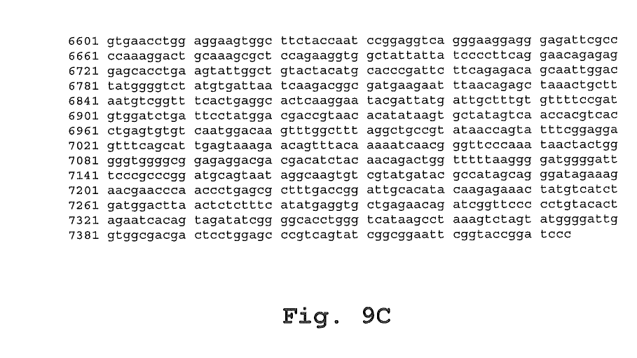

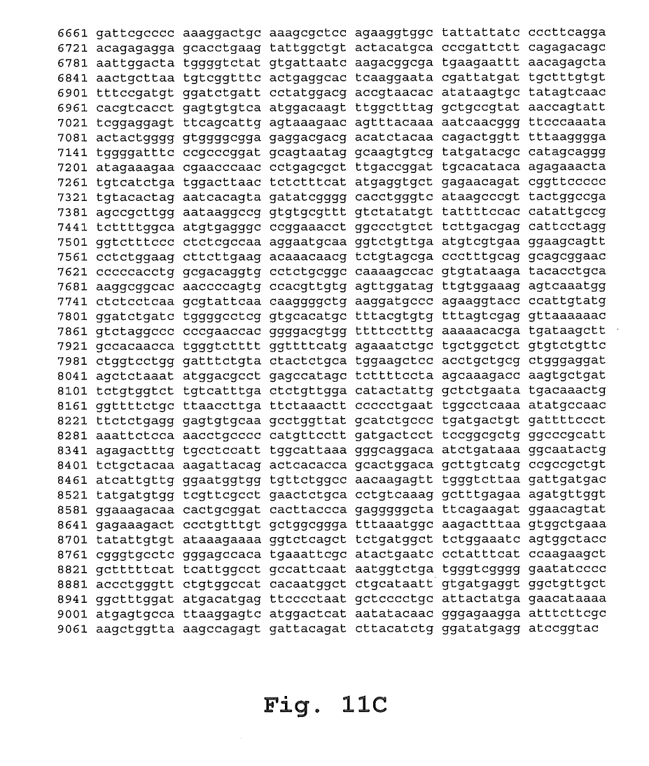

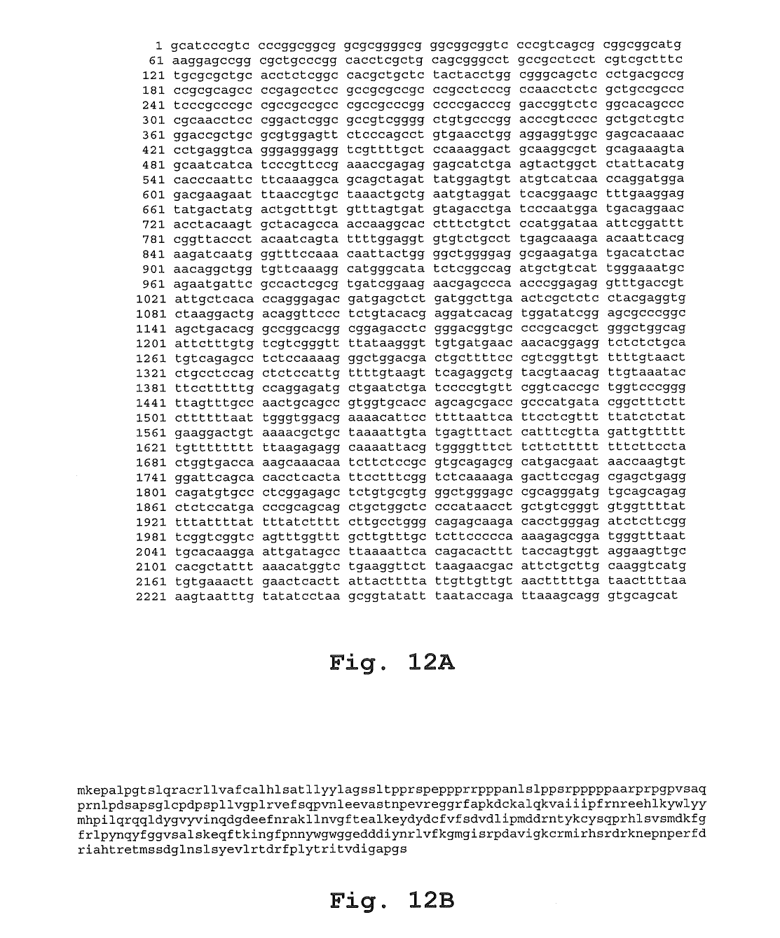

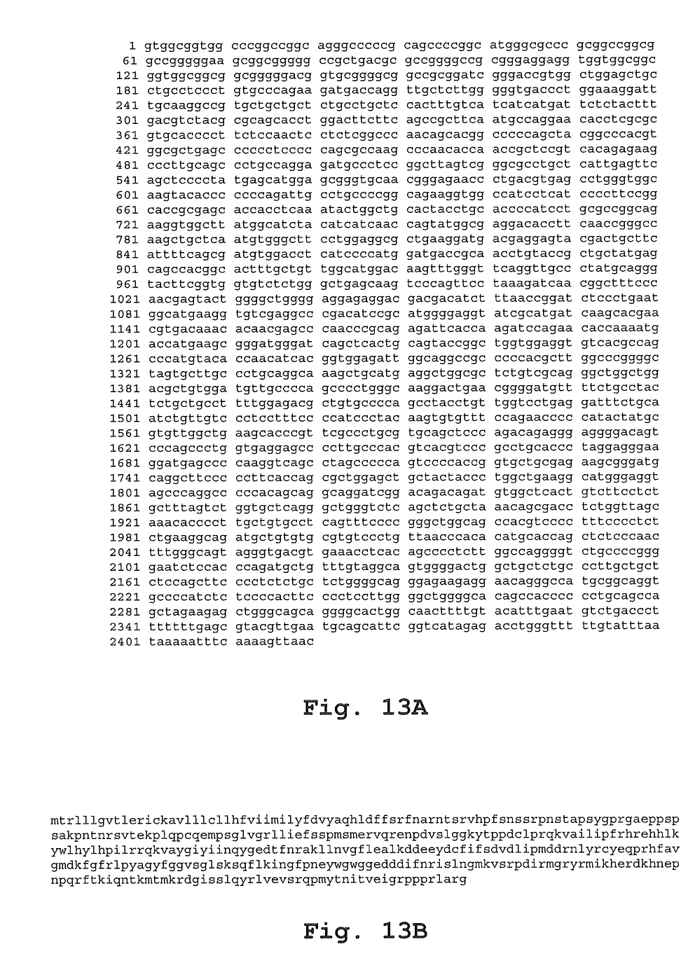

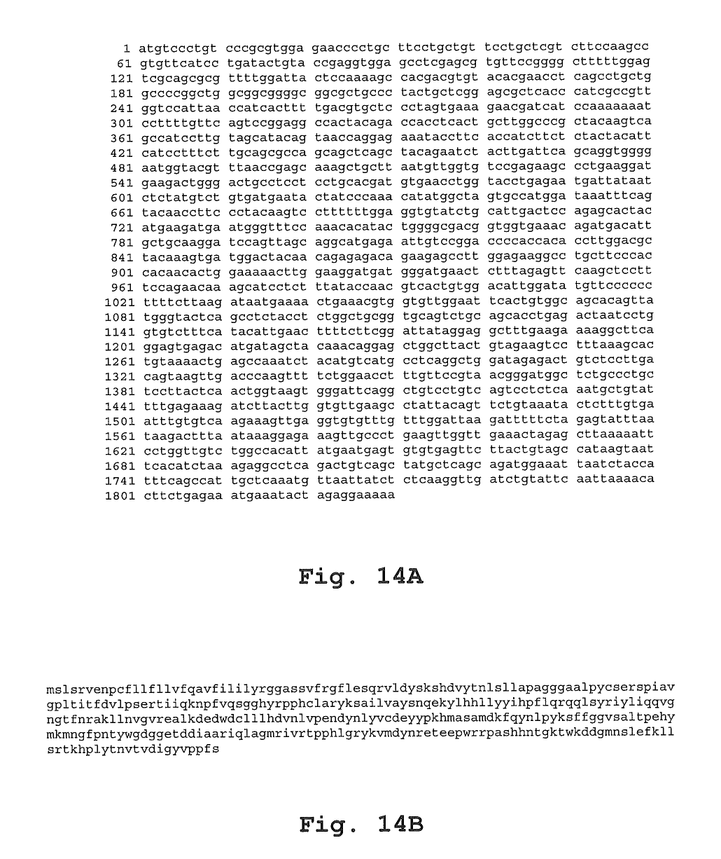

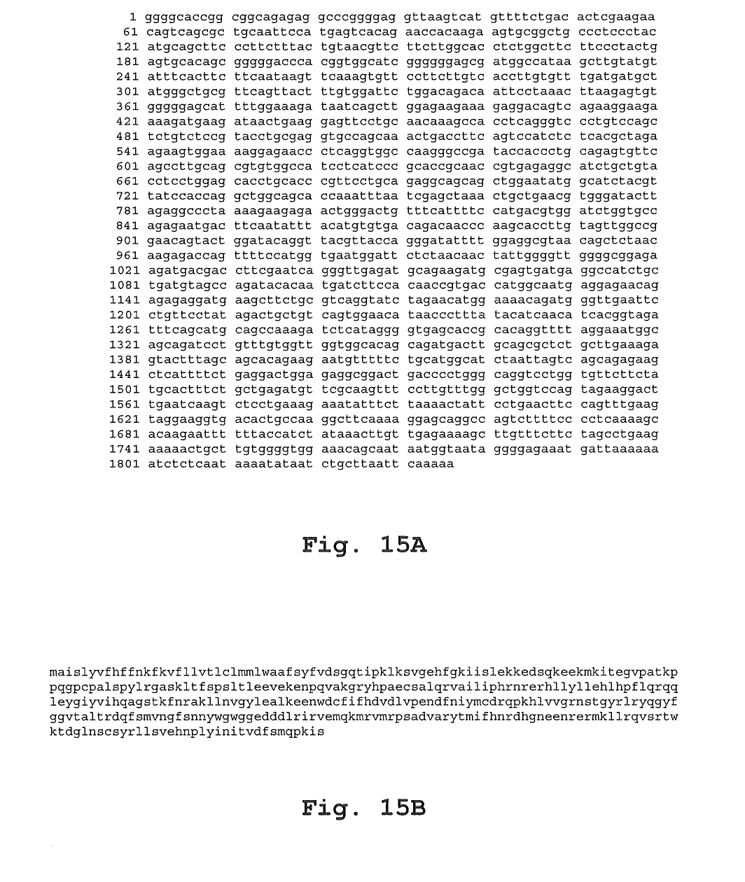

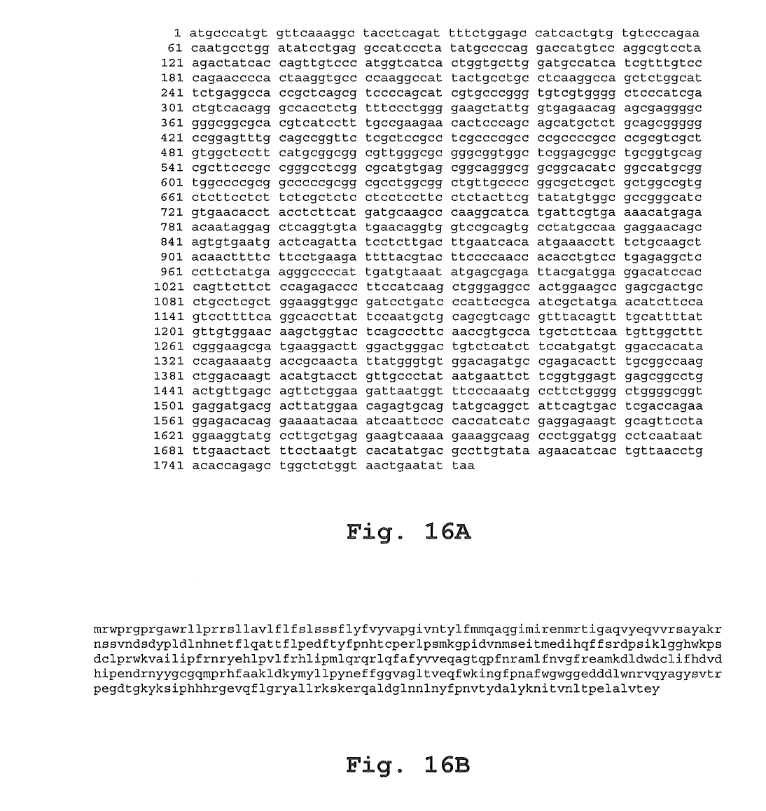

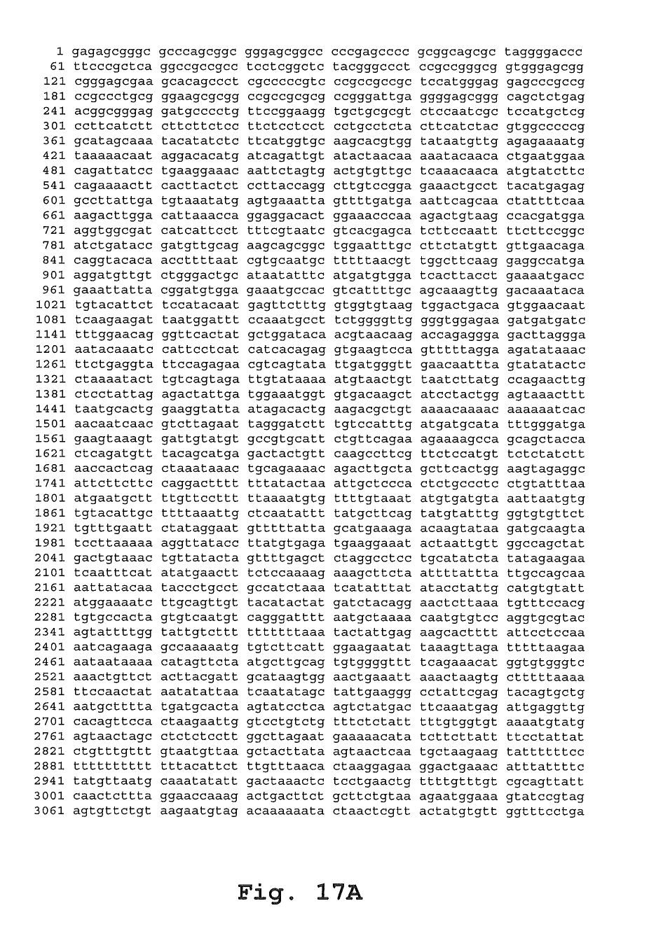

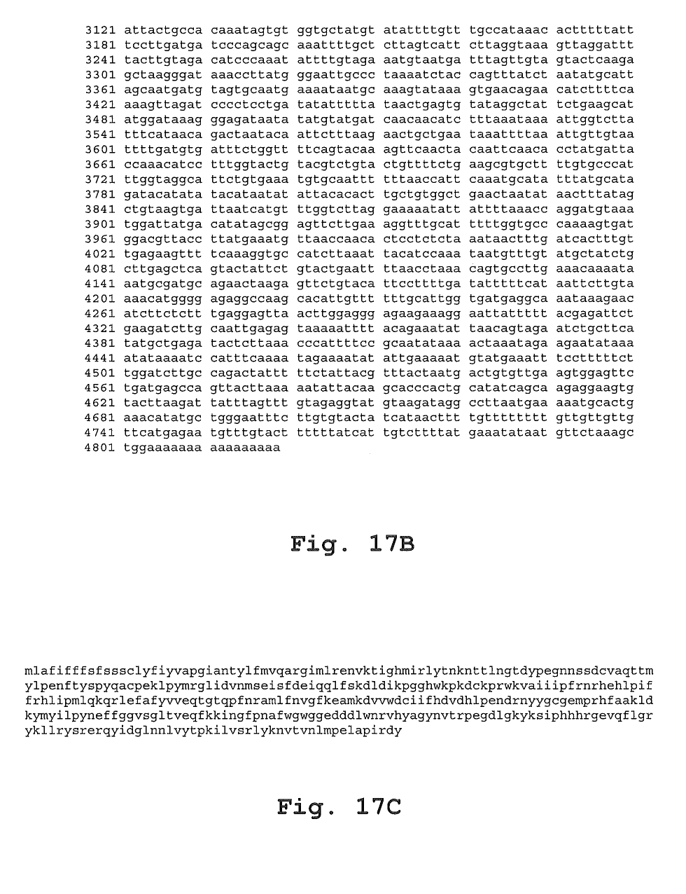

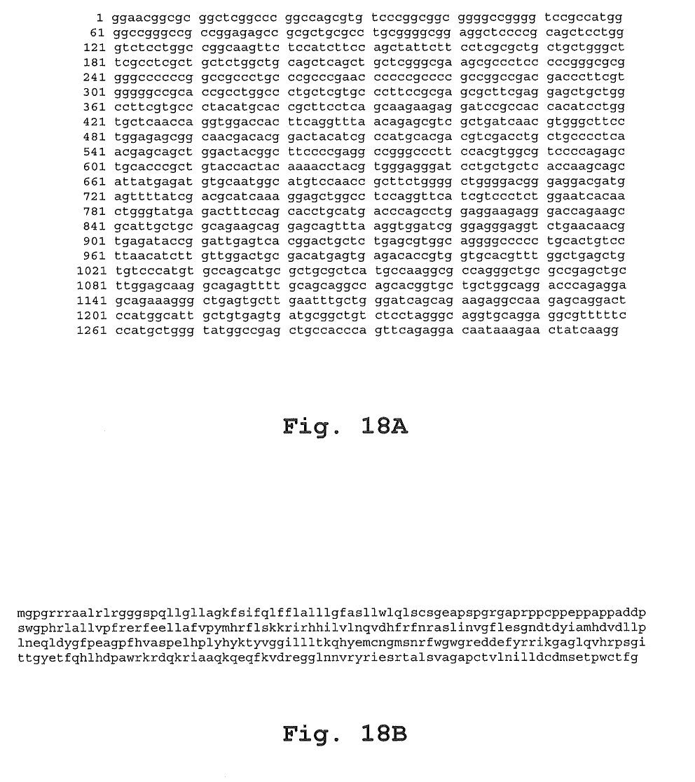

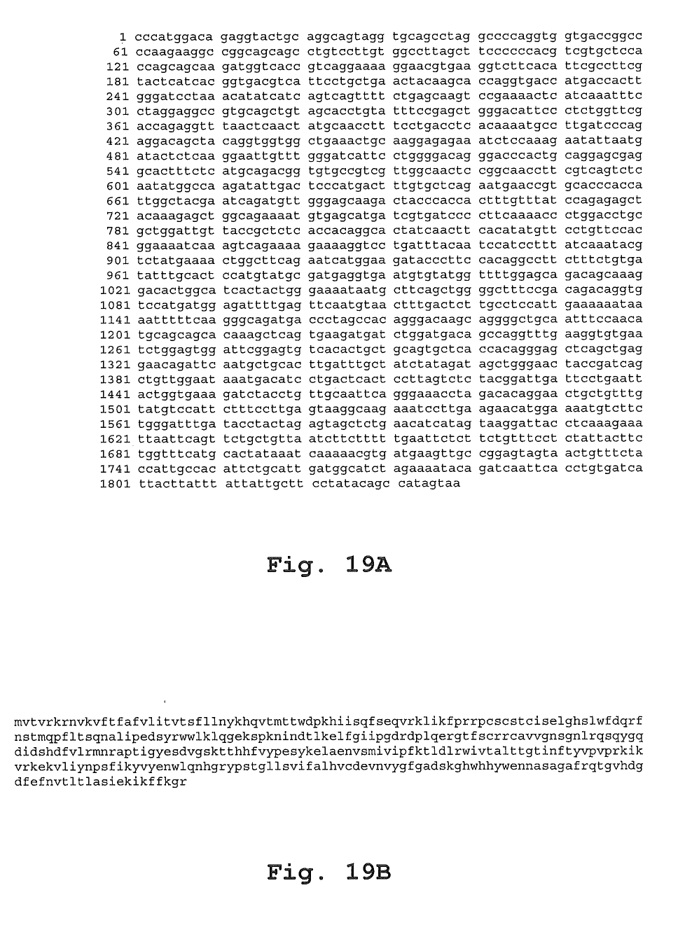

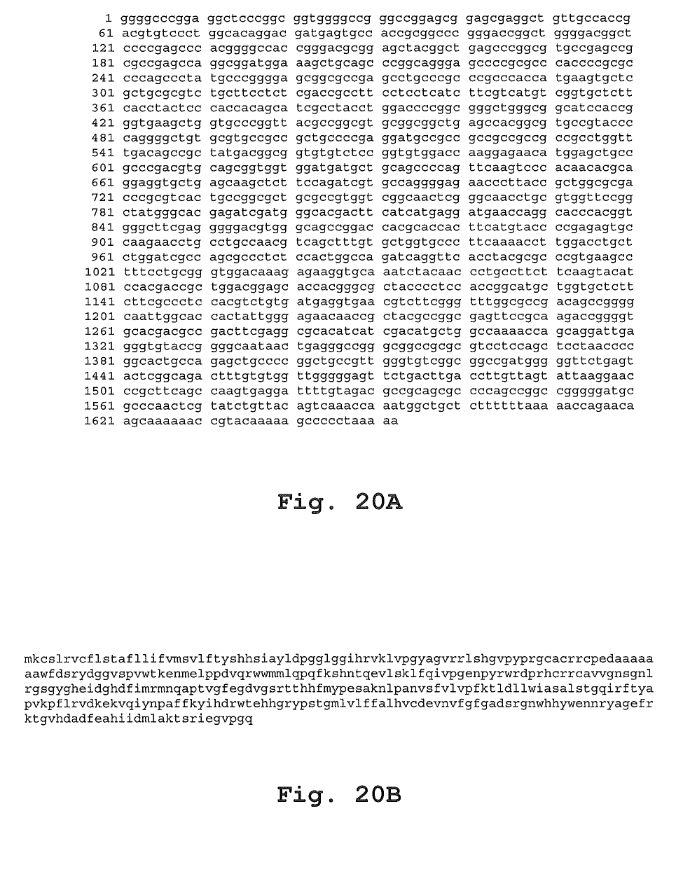

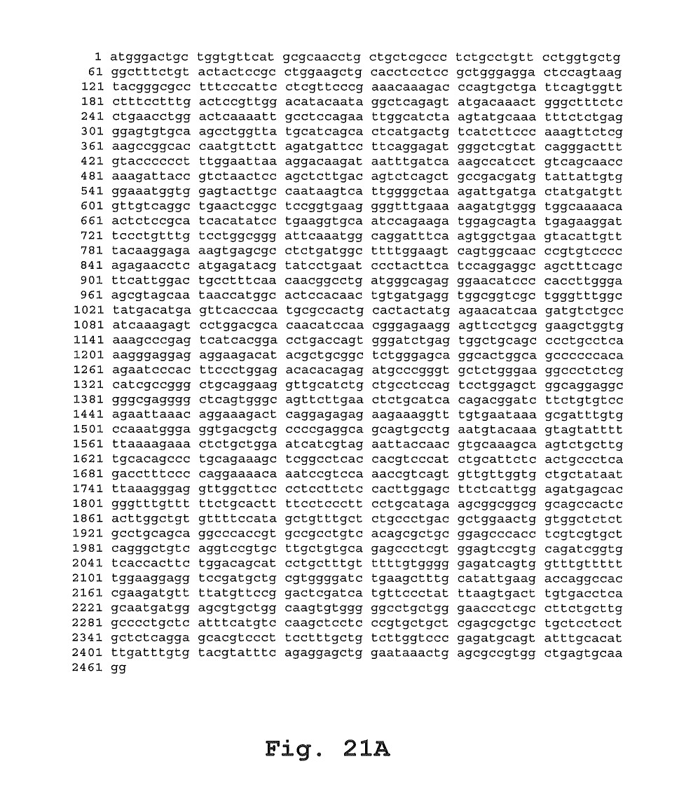

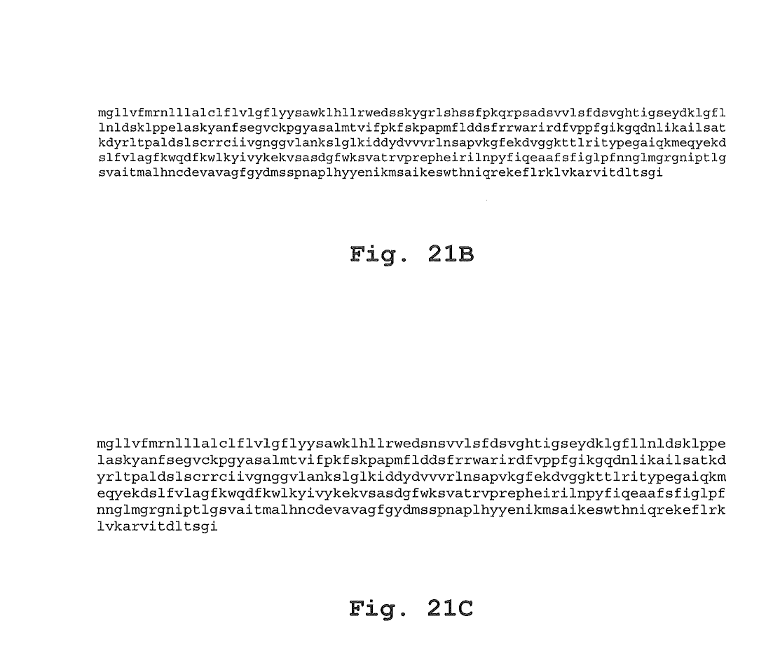

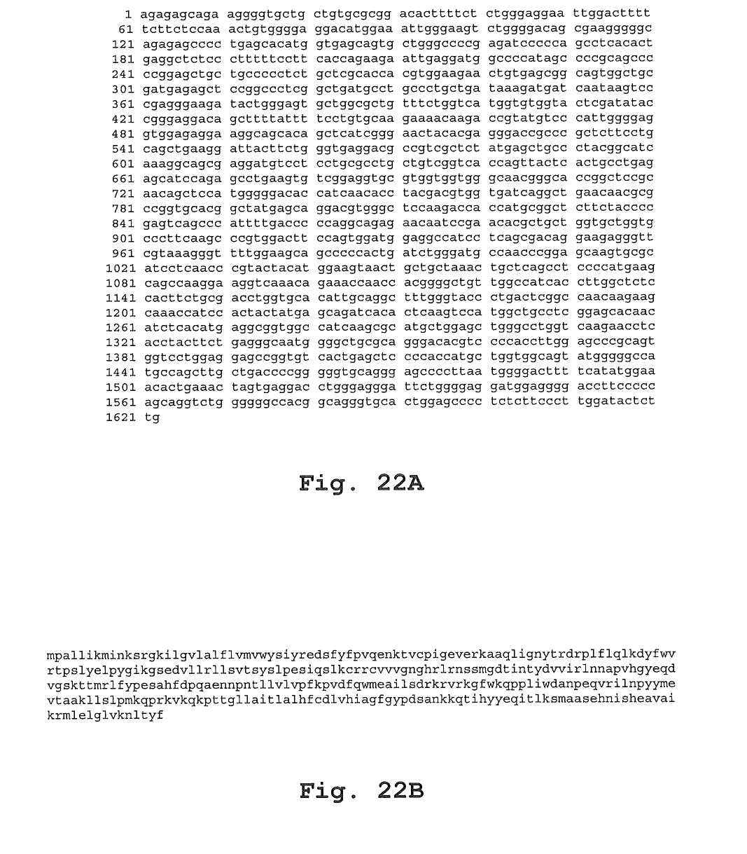

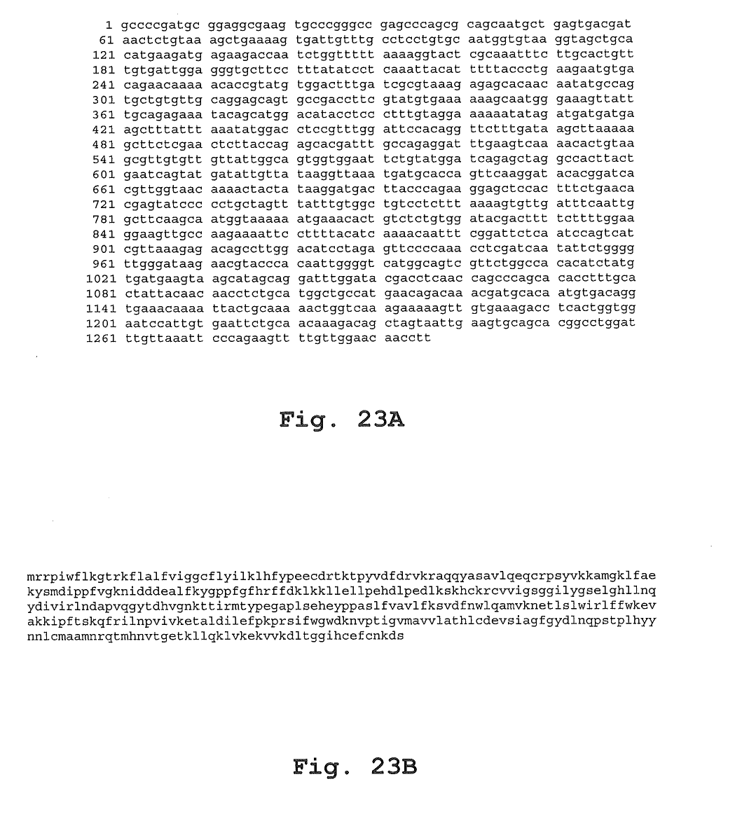

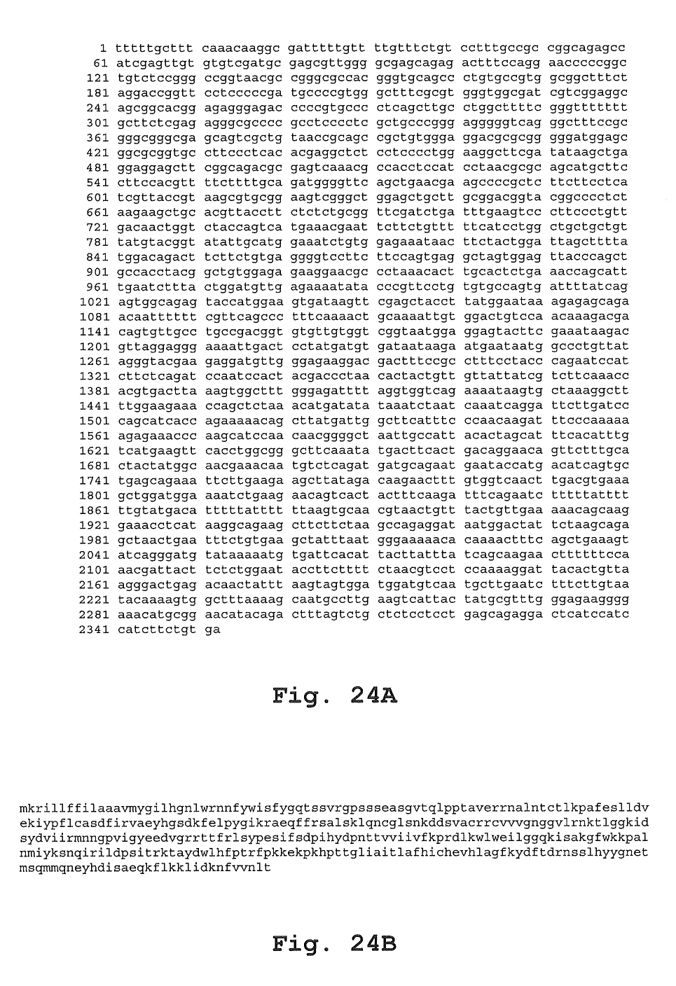

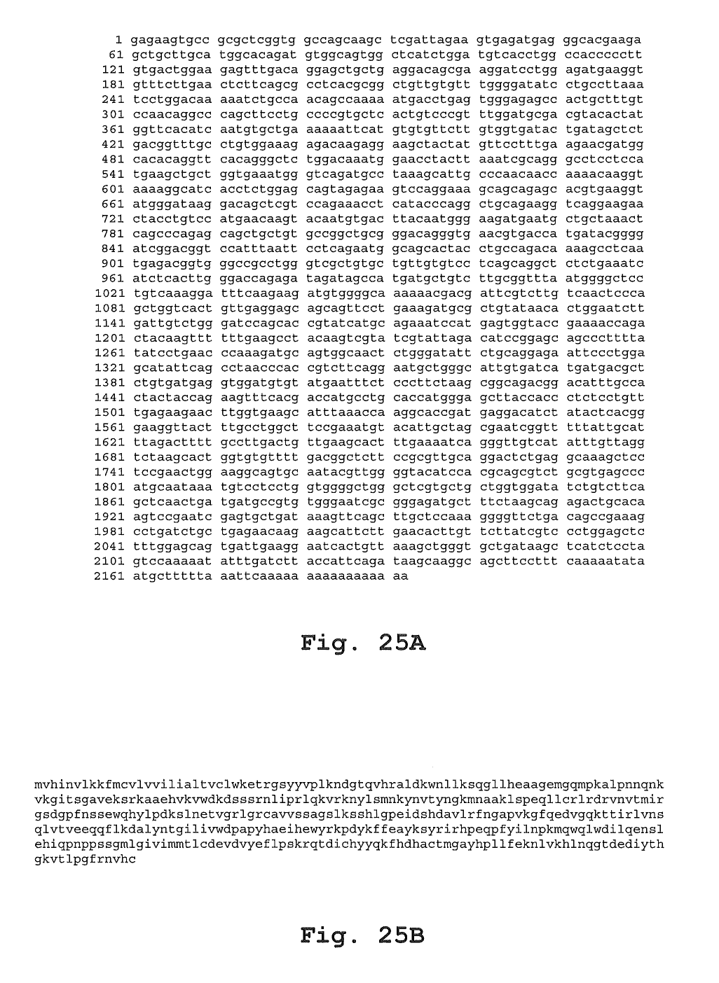

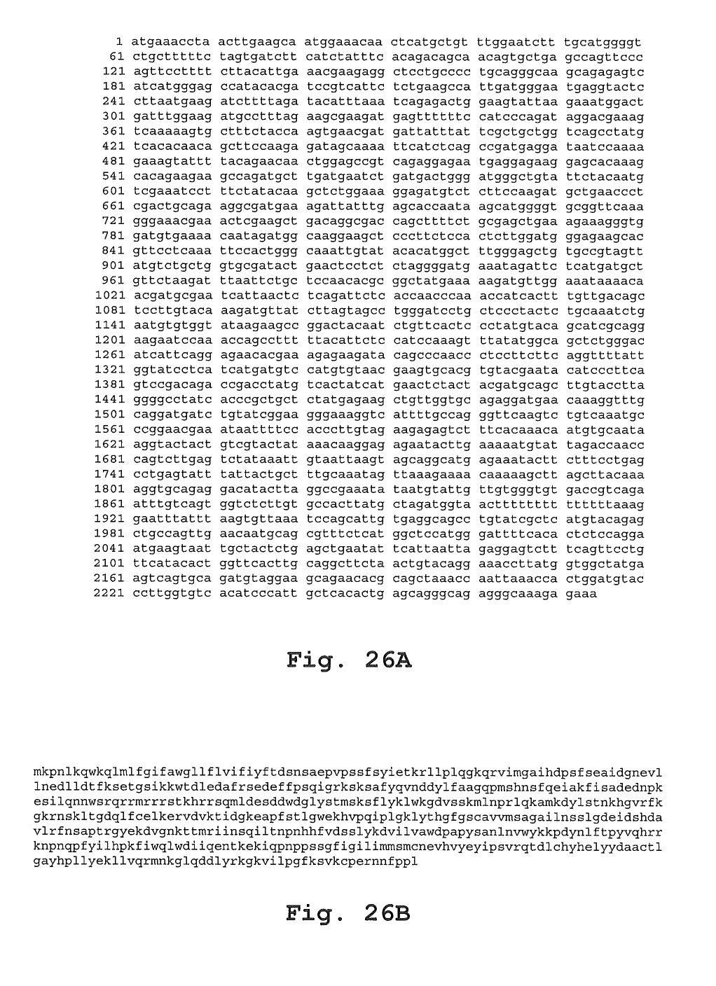

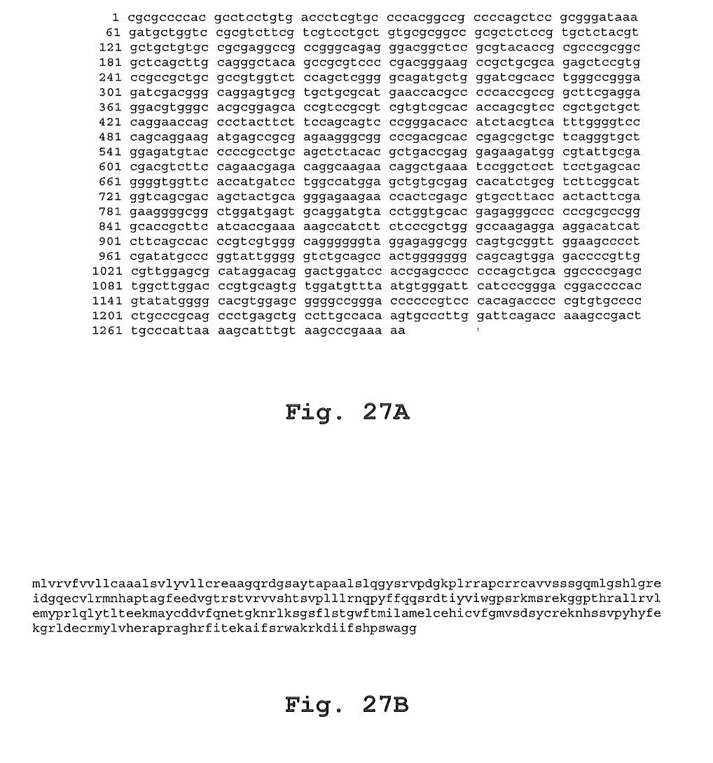

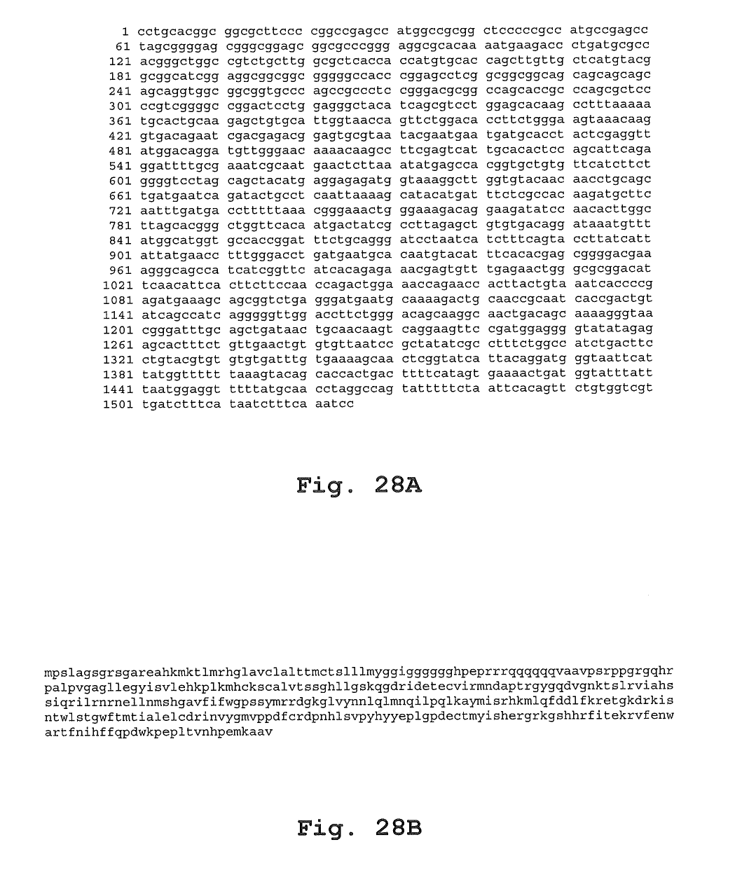

Nucleotide sequences of vectors shown at least in part in FIG. 6 are disclosed herein, for example, in FIGS. 9 to 11. Also shown are exemplary glycosyltransferase amino acid sequences and nucleotide sequences that encode glycosyltransferases, which are examples of those contemplated for use in accordance with the invention. Amino acid sequences which are 80%, 85%, 90%, 91%, 92%, 93%, 94%, 95%, 96%, 97%, 98% and 99% identical or homologous to each of the amino acid sequences disclosed herein including those disclosed in FIGS. 12 to 28 are also contemplated for use in accordance with the invention. Nucleotide sequences which are 80%, 85%, 90%, 91%, 92%, 93%, 94%, 95%, 96%, 97%, 98% and 99% identical or homologous to each of the nucleotide sequences disclosed herein, including those disclosed in FIGS. 9 to 28 are also contemplated for use in accordance with the invention.

Coding sequences are indicated for the glycosyltransferases disclosed herein and a practitioner of skill in the art can determine amino acid sequences from these specified coding sequences. Accordingly, the invention includes nucleotide sequences which will code for amino acid sequences which function as glycosyltransferases that are 80%, 85%, 90%, 91%, 92%, 93%, 94%, 95%, 96%, 97%, 98% and 99% identical to the amino acid sequence encoded by each of the glycosyltransferase coding sequences disclosed herein.

Also included within the scope of the invention is the use in accordance with the invention of functional fragments of each of the nucleotide and amino acid sequences disclosed herein.

The concepts and methods disclosed herein for adding sugars (e.g., sialic acid, galactose) to N-linked oligosaccharide structures of proteins are also contemplated for use in other animals and other organisms such as plants.

The present invention is further illustrated by the following examples, which are provided by way of illustration and should not be construed as limiting. The contents of all references, published patent applications and patents cited throughout the present application are hereby incorporated by reference in their entireties.

### Example 1

### Vector Design and Construction for Expression of GalT1 in the Avian Oviduct

The GalT1 coding sequence was synthesized by Integrated DNA Technologies (Coralville, Iowa) with codon usage optimized for expression in the hen magnum and is shown below (SEQ ID NO: 40):

ATGAAAGAACCTGCACTTCCTGGTACTTCACTGCAAAGAGCATG
TAGACTGCTGGTAGCATTTTGCGCCCTGCA
CCTGAGCGCAACCCTGCTCTACTACCTGGCTGGATCCAGCCTGA
CTCCACCCCGCTCTCCAGAACCTCCCCCTC
GGAGGCCGCCTCCAGCCAACCTCTCCCTGCCACCCTCCCGGCCT
CCTCCTCCCCCTGCGGCTCGCCCCCGCCCA
GGACCTGTTTCTGCACAACCCCGGAACCTGCCAGATTCTGCACC
ATCTGGACTGTGCCCCGATCCAAGTCCACT
GCTCGTTGGTCCTCTGCGGGTGGAGTTTAGTCAGCCAGTGAACCT
GGAGGAAGTGGCTTCTACCAATCCGGAGG
TCAGGGAAGGAGGGAGATTCGCCCCAAAGGACTGCAAAGCGCTCC
AGAAGGTGGCTATTATTATCCCCTTCAGG
AACAGAGAGGAGCACCTGAAGTATTGGCTGTACTACATGCACCCG
ATTCTTCAGAGACAGCAATTGGACTATGG
GGTCTATGTGATTAATCAAGACGGCGATGAAGAATTTAACAGAGC
TAAACTGCTTAATGTCGGTTTCACTGAGG
CACTCAAGGAATACGATTATGATTGCTTTGTGTTTTCCGATGTGG
ATCTGATTCCTATGGACGACCGTAACACAT
ATAAGTGCTATAGTCAACCACGTCACCTGAGTGTGTCAATGGACAA
GTTTGGCTTTAGGCTGCCGTATAACCAG
TATTTCGGAGGAGTTTCAGCATTGAGTAAAGAACAGTTTACAAAAAT
CAACGGGTTCCCAAATAACTACTGGGG
GTGGGGCGGAGAGGACGACGACATCTACAACAGACTGGTTTTTAAGG
GGATGGGGATTTCCCGCCCGGATGCA
GTAATAGGCAAGTGTCGTATGATACGCCATAGCAGGGATAGAAAGAA
CGAACCCAACCCTGAGCGCTTTGACCG
GATTGCACATACAAGAGAAACTATGTCATCTGATGGACTTAACTCTC
TTTCATATGAGGTGCTGAGAACAGATCG
GTTCCCCCTGTACACTAGAATCACAGTAGATATCGGGGCACCTGGG
TCATAA

The synthetic coding sequence was inserted into an ALV vector (gag, pol and env genes deleted) downstream of an ovomucoid (OM) promoter as shown in FIG. 6A (sequence shown in FIG. 9).

The LTRs of the ALV vector are self-inactivating (SIN) thus the vector is called pALV-SIN and is disclosed in US patent publication No. 2008/0064862, published Mar. 13, 2008, the disclosure of which is incorporated in its entirety herein by reference. The vector used is also an SC-negative vector as disclosed in US patent publication No. 2008/0064862. That is, elements associated with genes used for titering (i.e., the neomycin resistance gene) have been removed from pALV-SIN.

The pALV-SIN vector shown in FIG. 6A (FIG. 9) employs a 1.8 kb ovomucoid (OM) promoter which is used to drive magnum-specific expression of the galactosyltransferase coding sequence. The OM protein is one of the major egg white proteins and expression of the OM gene is essentially limited to the magnum. The vector is referred to as pALV-SIN-GalT1.

### Example 2

### Production of GalT1 Transgene Augmented Birds

The pALV-SIN-GalT1 vector produced as described in Example 1 was packaged into viral particles by the transient transfection method as disclosed in US patent publication No. 2007/0077650, published Apr. 5, 2007 (now U.S. Pat. No. 7,524,626).

Virus-containing media was collected 48 hr post-transfection and concentrated by centrifugation and immediately injected into stage X embryos of windowed eggs (stage X is an approximately 50,000 cell embryo, typically found in a freshly laid egg).

Approximately 150 embryos were injected. The eggs were sealed with a hot glue plug and incubated (Andacht, et al. Mol Reprod Dev 69: 31-4, 2004). 42 chicks hatched about 21 days later and the blood DNA was assessed for the presence of the transgene one week later. The hatched chicks are designated G0 for generation zero.

To assess the success of the transgenesis procedure, a Taqman® quantitative PCR system was used to determine transgene content in the blood DNA of hatched G0 chicks (Harvey, et al. Poultry Science 81: 202-12, 2002). Primers and a probe tagged with fluorescent labels were designed based on the sequence of the glycosyltransferase CDS. Blood DNA was purified, quantitated by the Picogreen® kit and analyzed with Taqman assay. About 80% of the chicks had detectable levels of the transgene in their blood DNA.

Further analysis was performed to confirm that the transgene integrated intact. PCR primers were used to amplify various parts of the transgene (the OM promoter, the CDS and 3′ untranslated region) from the blood DNA of positive chicks and the sizes of the PCR products were determined by agarose gel electrophoresis. All GalT1 positive G0 birds that were tested were found to contain intact copies of the transgene.

### Example 3

### Production of Fully Transgenic GalT1 Birds And Assessment Of Transgene-Augmented Glycosylation

Semen was collected from G0 roosters of Example 2 and sperm DNA was analyzed by the Taqman assay for transgene content (Harvey, Speksnijder, Baugh, Morris and Ivarie. Poultry Science 81: 202-12, 2002). Roosters with the highest transgene content were bred to wild-type hens and offspring were analyzed by Taqman to identify fully transgenic G1s.

Eggs were collected from 13 G1 hens. Egg white proteins were treated with PNGase which specifically releases N-linked oligosaccharides (N-glycans) from proteins. The N-glycans were purified and structures were determined by MALDI-MS analysis, the results of which are shown in FIG. 8. As can be seen, the results demonstrate the effectiveness of the invention with a substantial amount of galactose being added to many of the oligosaccharide structures. In addition, FIGS. 8A-8C shows that more sialic acid has been added to the oligosaccharide structures of the protein of the GalT1 birds relative to the oligosaccharide structures of the protein from the wild type (FIG. 8D) hen.

### Example 4

### Vector Design and Construction for Expression of SialT3 Transgene Augmented Birds

The SialT3 coding sequence has been synthesized with codon usage optimized for expression for Synthetic Chicken α-2,3-Sialyltransferase Type 3 in the hen magnum and is shown below (SEQ ID NO: 41):

ATGGGTCTTTTGGTTTTCATGAGAAATCTGCTGCTGGCTCTGTGTCT
GTTCCTGGTCCTGGGATTTCTGTACTAC
TCTGCATGGAAGCTCCACCTGCTGCGCTGGGAGGATAGCTCTAAATAT
GGACGCCTGAGCCATAGCTCTTTTCC
TAAGCAAAGACCAAGTGCTGATTCTGTGGTTGTCATTTGACTCTGTT
GGACATACTATTGGCTCTGAATATGA
CAAACTGGGTTTTCTGCTTAACCTTGATTCTAAACTTCCCCCTGAATT
GGCCTCAAAATATGCCAACTTCTCTGA
GGGAGTGTGCAAGCCTGGTTATGCATCTGCCCTGATGACTGTGATTTT
CCCTAAATTCTCCAAACCTGCCCCCAT
GTTCCTTGATGACTCCTTCCGGCGCTGGGCCCGCATTAGAGACTTTGT
GCCTCCATTTGGCATTAAAGGGCAGG
ACAATCTGATAAAGGCAATACTGTCTGCTACAAAAGATTACAGACTC
ACACCAGCACTGGACAGCTTGTCATGC
CGCCGCTGTATCATTGTTGGGAATGGTGGTGTTCTGGCCAACAAGAG
TTTGGGTCTTAAGATTGATGACTATGA
TGTGGTCGTTCGCCTGAACTCTGCACCTGTCAAAGGCTTTGAGAAAGAT
GTTGGTGGAAAGACAACACTGCGGA
TCACTTACCCAGAGGGGGCTATTCAGAAGATGGAACAGTATGAGAAAG
ACTCCCTGTTTGTGCTGGCGGGATTT
AAATGGCAAGACTTTAAGTGGCTGAAATATATTGTGTATAAAGAAAAG
GTCTCAGCTTCTGATGGCTTCTGGAAA
TCAGTGGCTACCCGGGTGCCTCGGGAGCCACATGAAATTCGCATACTG
AATCCCTATTTCATCCAAGAAGCTGC
TTTTTCATTCATTGGCCTGCCATTCAATAATGGTCTGATGGGTCGGGGG
AATATCCCCACCCTGGGTTCTGTGGC
CATCACAATGGCTCTGCATAATTGTGATGAGGTGGCTGTTGCTGGCTTTG
GATATGACATGAGTTCCCCTAATGC
TCCCCTGCATTACTATGAGAACATAAAAATGAGTGCCATTAAGGAGTCA
TGGACTCATAATATACAACGGGAGA
AGGAATTTCTTCGCAAGCTGGTTAAAGCCAGAGTGATTACAGATCTTAC
ATCTGGGATATGA

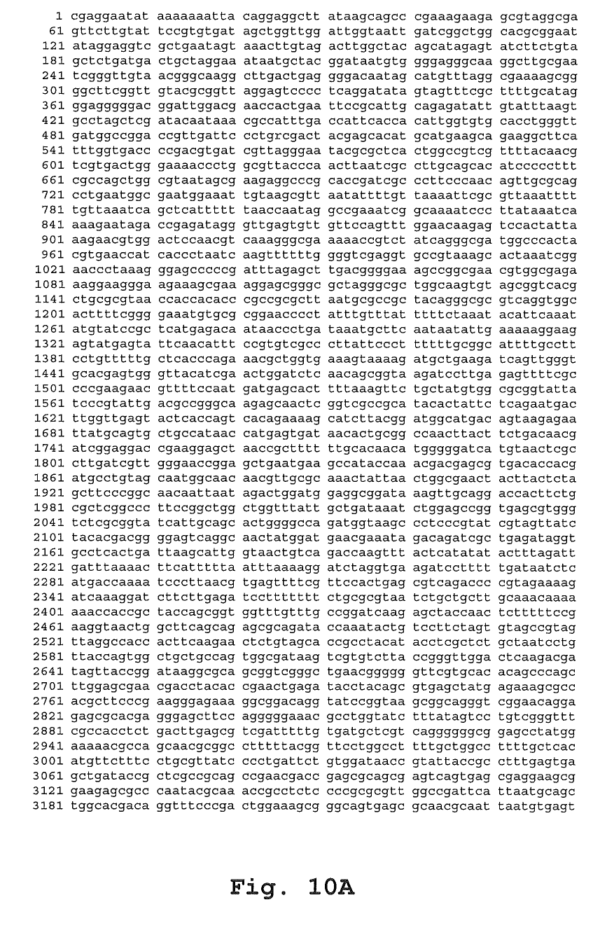

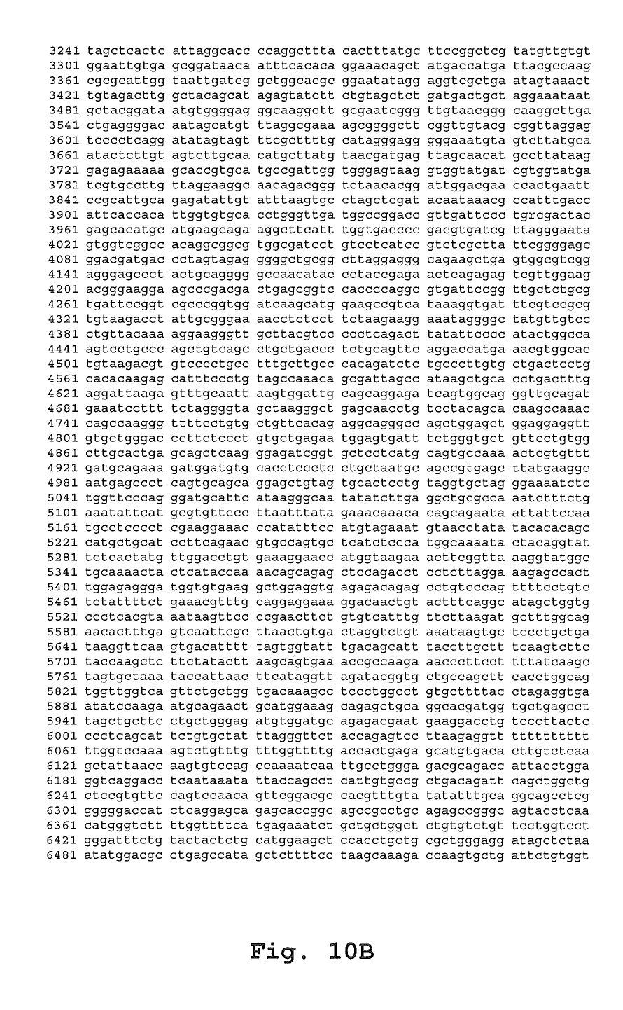

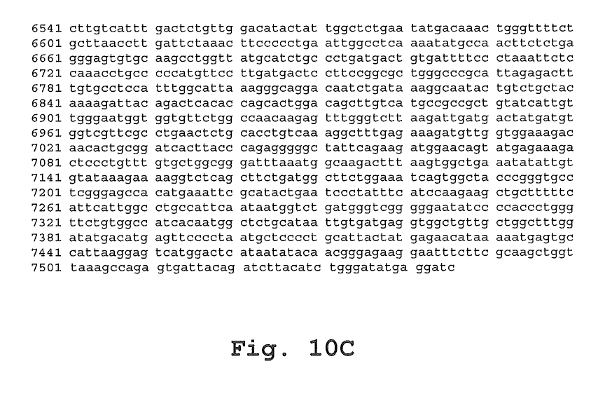

The synthetic coding sequence was inserted into a pALV-SIN vector downstream of an ovomucoid (OM) promoter as shown in FIG. 6B to produce pALV-SIN-SialT3, sequence shown in FIG. 10. The construct is assembled and G0 birds are then produced and analyzed essentially as described for the GalT1 G0 birds in Examples 1 and 2 and G1 birds produced essentially as described for the GalT1 birds in Example 3.

### Example 5

### Production of SialT3/GalT1 Transgene Augmented Birds by Crossing SialT3 Positive Birds and GalT1 Positive Birds

One or more of the GalT1 G1 birds of Example 3 (or a homozygous G2 GalT1 bird obtained from crossing two GalT1 G1 birds) is crossed with a SialT3 G1 bird of Example 4 (or crossed with a homozygous G2 SialT3 bird obtained from crossing two SialT3 G1 birds) such that the resulting offspring birds carry both the GalT1 and SialT3 transgenes, as is understood in the art. These birds can be crossed to each other a second time to produce birds that are homozygous for both transgenes, as is understood in the art.

### Example 6

### Vector Design and Construction for Production of SialT3 and GalT1 Transgene Augmented Birds Using a Single Expression Vector

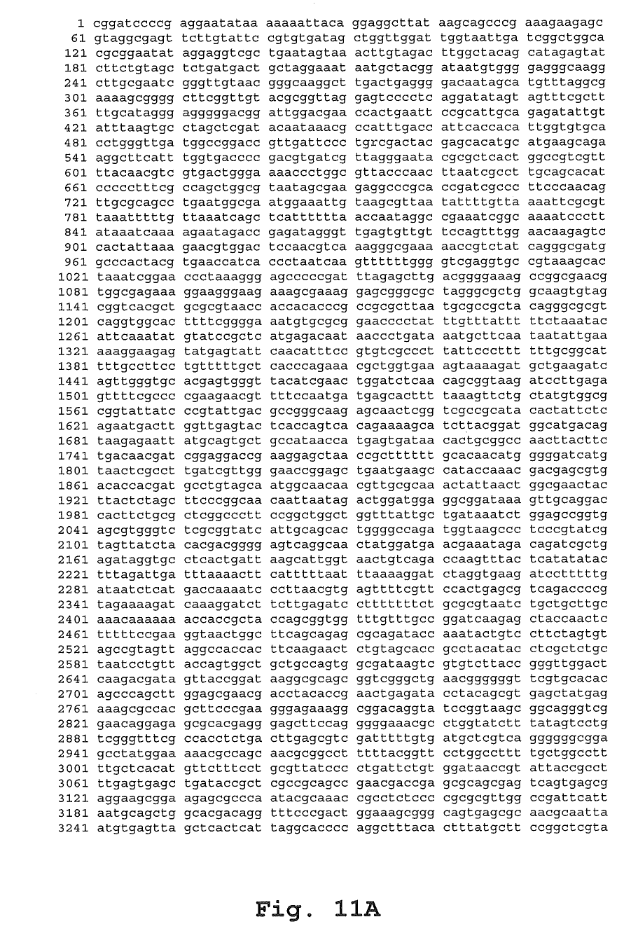

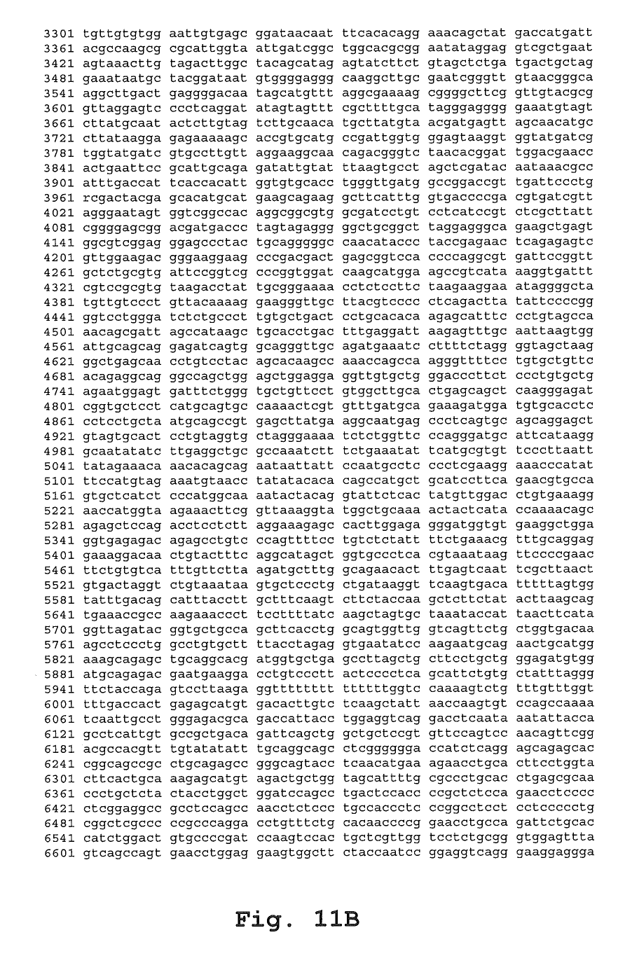

The GalT1 and SialT3 coding sequences are synthesized with codon usage optimized for expression in the hen magnum as in Example 1 and Example 4. The coding sequences are inserted into a single retroviral vector downstream of a single 1.8 kb ovomucoid promoter. A sequence (e.g., an IRES) which provides for translation of the second or downstream CDS is inserted between the GalT1 and SialT3 CDSs, thus producing a vector having a bicistronic message as shown in FIG. 6C and in FIG. 11.

Translation of GalT1 is initiated by the upstream translation initiation site and SialT3 translation is initiated by the internal ribosome entry site (IRES) and accordingly both the GalT1 and SialT3 CDSs are expressed from the same mRNA. The IRES in FIG. 6C is from the encephalomyocarditis virus (EMCV) (Jang, et al. J Virol 62: 2636-43, 1988; Ghattas, et al. Mol Cell Biol 11: 5848-59, 1991).

The vector is inserted into avian (e.g., chicken, quail, turkey) embryos and G0s and G1s are obtained essentially as described in the Examples above for pALV-SIN-GalT1. Homozygotes can be obtained as is understood in the art.

All references cited herein are incorporated by reference herein in their entirety and for all purposes to the same extent as if each individual publication, patent or patent application is specifically and individually indicated to be incorporated by reference in its entirety for all purposes.

The citation of any publication is for its disclosure prior to the filing date and should not be construed as an admission that the present invention is not entitled to antedate such publication by virtue of prior invention.

While this invention has been described with respect to various specific examples and embodiments, it is to be understood that the invention is not limited thereto and that it can be variously practiced with the scope of the following claims.

What is claimed is:

1. A transgenic chicken whose genome comprising a transgene encoding sialyltransferase operably linked to a promoter, wherein the sialyltransferase is expressed in oviduct tissue of the chicken, and wherein the sialyltransferase adds terminal sialic acid on oligosaccharides of proteins produced in the oviduct tissue of the chicken.

2. The transgenic chicken of claim 1 wherein the oviduct tissue is magnum tissue.

3. The transgenic chicken of claim 1 wherein the oviduct tissue comprises tubular gland cells.

4. The transgenic chicken of claim 1 wherein the sialyltransferase is sialyltransferase type 1, 2, 3, 4, 5 or 6.

5. The transgenic chicken of claim 4 wherein the wherein the sialyltransferase is sialyltransferase type 1.

6. The transgenic chicken of claim 4 wherein the sialyltransferase is sialyltransferase type 2.

7. The transgenic chicken of claim 4 wherein the sialyltransferase is sialyltransferase type 3.

8. The transgenic chicken of claim 4 wherein the sialyltransferase is sialyltransferase type 4.

9. The transgenic chicken of claim 4 wherein the sialyltransferase is sialyltransferase type 5.

10. The transgenic chicken of claim 4 wherein the sialyltransferase is sialyltransferase type 6.

11. The transgenic chicken of claim 1 wherein the promoter is an oviduct-specific promoter.

12. The transgenic chicken of claim 11 wherein the oviduct-specific promoter is an ovalbumin promoter.

13. The transgenic chicken of claim 11 wherein the oviduct-specific promoter is an ovomucoid promoter.

14. The transgenic chicken of claim 1 wherein the proteins are secreted into egg white of the chicken.
